(19) United States
(12) Patent Application Publication
(10) Pub. No.: US 2025/0136704 A1
(43) Pub. Date: May 1, 2025

(54) METHODS FOR ADMINISTERING A BCMAXCD3 BINDING MOLECULE

(71) Applicant: AMGEN INC., Thousand Oaks, CA (US)

(72) Inventors: Anjali Sharma, Thousand Oaks, CA (US); Zachariah Mclver, Thousand Oaks, CA (US); Bhakti Mehta, Thousand Oaks, CA (US)

(21) Appl. No.: 18/035,054

(22) PCT Filed: Nov. 10, 2021
(86) PCT No.: PCT/US21/58695

§ 371 Date: May 2, 2023

(51) Int. Cl. C07K 16/28; A61K 9/00; A61K 39/00; A61P 35/00

(52) U.S. Cl. CPC C07K 16/2878; A61K 9/0019; A61P 35/00; C07K 16/2809; A61K 2039/505; A61K 2039/54; A61K 2039/545; C07K 2317/31; C07K 2317/524; C07K 2317/526; C07K 2317/565; C07K 2317/76; C07K 2317/94

(57) ABSTRACT

The present invention relates to the dosage and administration of anti-BCMA x anti-CD3 binding molecules for the treatment of BCMA positive neoplasms. More specifically, the present invention relates to a protein comprising a first domain which binds to BCMA, a second domain which binds to CD3 and a third domain which enhances the half-life of the protein, for use in the treatment or amelioration of a BCMA positive neoplasm, wherein the protein is administered at a specified dose regimen in at least one cycle. Moreover, the invention relates to a method for the treatment or amelioration of a BCMA positive neoplasm comprising administering a specified dose regimen of such binding molecule, to methods for administering therapeutic doses of such binding molecules and to the use of such binding molecules for the manufacture of a medicament for the treatment or amelioration of a BCMA positive neoplasm.

### INCORPORATION BY REFERENCE OF THE SEQUENCE LISTING

This application includes a sequence listing submitted electronically as text file I-55257_SeqListing.txt; Size: 705,285 bytes; Created: Dec. 27, 2023 and is incorporated herein by reference.

### FIELD OF THE INVENTION

The present invention relates to the dosage and administration of anti-BCMA x anti-CD3 binding molecules for the treatment of BCMA positive neoplasms. More specifically, the present invention relates to a protein comprising a first domain which binds to BCMA, a second domain which binds to CD3 and a third domain which enhances the half-life of the protein, for use in the treatment or amelioration of a BCMA positive neoplasm, wherein the protein is administered at a specified dose regimen in at least one cycle. Moreover, the invention relates to a method for the treatment or amelioration of a BCMA positive neoplasm comprising administering a specified dose regimen of such binding molecule, to methods for administering therapeutic doses of such binding molecules and to the use of such binding molecules for the manufacture of a medicament for the treatment or amelioration of a BCMA positive neoplasm.

### BACKGROUND OF THE INVENTION

Multiple Myeloma (MM) is a malignant tumor of plasma cells which proliferate in bone marrow and release para protein. The resulting clinical laboratory pictures include infection, bone destruction, bone marrow failure, renal failure and hypercalcemia. The age adjusted annual incidence is increasing with approximately 6 new cases per 100,000. The incidence is 2 times higher in the black US population than in Caucasians. The 5-year survival rate for MM has increased from ˜ 25% for newly diagnosed patients in 1975 to ˜ 45% in 2006. This improvement is mainly due to new drugs such as proteasome inhibitors and immunomodulators (IMiDs). However, MM is not considered curable with current approaches. Patients refractory to proteasome inhibitors and immunomodulators show an unfavorable outcome with a median overall survival of 9 months.

Outcome is particularly poor in high-risk populations such as the subgroup with del17p13 positive MM. Although many drugs are in clinical development for MM, new treatment options are still needed. Patients showing symptomatic disease are initially treated with primary induction therapy followed by high dose chemotherapy with autologous stem cell support in eligible patients. Patients eligible for intensive treatment are determined by age (65 to 75 years as upper limit), no comorbidities and intact renal function. Although this regimen has improved survival of younger and fit patients, the median duration of response does not exceed 3 years, and few patients remain free of the disease for more than 10 years.

Consolidation and maintenance approaches have been tested in order to increase the depth and duration of remission. Because maintenance treatment is challenging due to no efficacy or tolerability, there is still the option of improving survival outcome in the transplant setting by adding novel treatments to induction, consolidation or maintenance regimens. Patients not eligible for high dose therapy commonly receive induction regimens similar to the ones for the transplant candidates. These regimens include the proteasome inhibitor Bortezomib or a Melphalan based combination with Thalidomide. Median overall survival (OS) of Melphalan-Thalidomide-Prednisone (MPT) in elderly patients is 40 months. Lenalidomide in combination with Dexamethasone is the standard regimen for relapsed/refractory MM, but may move to the first line setting in transplant ineligible patients.

Other established regimens in the relapsed setting are repeat induction regimens or bortezomib or immunomodulator based salvage combinations with alkylating agents. An improvement of outcome (progression-free survival (PFS) and OS) is needed for patients in relapsed disease. Patients which are refractory to established treatments and progressing on treatment have a dismal outcome of 9 months OS on treatment and 3 months without treatment. The unmet need is highest in these patients.

Bispecific molecules such as BiTE® (“Bispecific T cell Engager”) molecules are recombinant proteins comprising one binding domain that is specific for a selected tumor-associated surface antigen on target cells, and a second binding domain that is specific for CD3, a subunit of the T cell receptor complex on T cells. By their particular design, BiTE® molecules are uniquely suited to transiently connect T cells with target cells and, at the same time, potently activate the inherent cytolytic potential of T cells against target cells. The first generation of so-called “canonical” (non-half-life-extended) BiTE® molecules (see WO 99/54440 and WO 2005/040220) was brought into the clinic as AMG 103 (blinatumomab, anti-CD19 x anti-CD3) and AMG 110 (solitomab, anti-EpCAM x anti-CD3). A further development of the first generation of canonical BiTE® molecules was the provision of bispecific polypeptides binding to a context independent epitope at the N-terminus of the CD3-epsilon chain of human and Callithrix jacchus, Saguinus oedipus or Saimiri sciureus (WO 2008/119567). The first BITE® molecule comprising this new CD3-epsilon binding domain that was tested in the clinic was AMG 330.

Bispecific polypeptides as described in WO 2008/119567 are likely to suffer from rapid clearance from the body; thus, whilst they are able to reach most parts of the body rapidly, and are quick to produce and easy to handle, their in vivo applications may be limited by their brief persistence in vivo. Prolonged administration by continuous intravenous infusion was used to achieve therapeutic effects because of the short in vivo half-life of this relatively small molecule. However, such continuous intravenous infusions are classified as inconvenient for the patients and, thus, in case of more convenient alternative treatment approaches, might hamper the election of the compound demonstrated to be more efficient in the treatment of the respective disease. This led to the development of bispecific therapeutics that retain similar therapeutic efficacy and at the same time have favorable pharmacokinetic properties, including a longer half-life. Therefore, an important further development of the so-called canonical BiTE® molecules was the addition of a further domain which extends or enhances the half-life of the polypeptide. The resulting molecules are also called “HLE” (half-life extended) BiTE® molecules, see e.g. WO 2017/134140.

B cell maturation antigen (BCMA, TNFRSF17, CD269) is a transmembrane protein belonging to the TNF receptor super family. BCMA expression is selectively induced during late stage plasma cell differentiation and is absent on naïve and memory B cells. Upon BCMA binding to its ligands, B cell activating factor (BAFF) and a proliferation inducing ligand (APRIL), the survival of the bone marrow plasma cells and plasmablasts is promoted. BCMA does not maintain normal B cell homeostasis, but is required for the survival of long lived plasma cells. Studies in BCMA −/− mice showed that the survival of long-lived bone marrow plasma cells was impaired, but B cell development and early humoral immune responses were indistinguishable from wild type mice. The mRNA expression of BCMA is highly elevated in malignant plasma cell disorders. By contrast, mRNA expression in normal tissues is very low and restricted to lymphoid tissues where normal long-lived plasma cells are located. BCMA protein expression is reported to be restricted to plasma cells only. Expression of BCMA is confined to plasma blasts and long-lived plasma cells and cannot be detected on other normal human tissues. BCMA is universally expressed on the cell surface of MM cells and at relatively higher levels on malignant plasma cells than the level observed on normal plasma cells. There is no correlation between BCMA expression and MM disease stage, response to last treatment and time from diagnosis. Neither T cells nor myeloid cells or CD34+ hematopoietic stem cells express BCMA. The selective expression of BCMA makes it a very attractive target for antibody-based and chimeric antigen receptor (CAR)-based therapies.

AMG 420 (formerly BI 836909) is a canonical bispecific T cell engager which binds to BCMA on target cells as well as to CD3-epsilon on T cells. It functions as a bridge between BCMA positive target cells such as MM cells and cytotoxic T lymphocytes (CTLs) by directing the cytolytic activity of CTLs to the target cells. AMG 420 consists of two single chain variable fragments (scFv), one being directed to BCMA and the other one to CD3. Each of the scFv fragments consists of a VH and a VL domain connected with a glycine/serine linker. The two scFv fragments are also connected with a glycine/serine linker. Half-life extended anti-BCMA x anti-CD3 polypeptides such as AMG 701 are described in detail in WO 2017/134134. There is an ongoing unmet medical need for the development of therapies for the treatment of BCMA positive neoplasms, in particular with such half-life extended anti-BCMA x anti-CD3 molecules.

### SUMMARY OF THE INVENTION

Based on the disclosure provided herein, those skilled in the art will recognize, or be able to ascertain using no more than routine experimentation, many equivalents to the specific embodiments of the invention described herein. Such equivalents are intended to be encompassed by the following items.

Item 1 A protein comprising a first domain which binds to BCMA, a second domain which binds to CD3 and a third domain which extends the half-life of the protein, for use in the treatment or amelioration of a BCMA positive neoplasm, wherein the protein is administered in a first cycle comprising:
administering a first dose of the protein on day 1, and
administering a second dose of the protein on a day after day 1 and before day 8, wherein the second dose exceeds the first dose and is at least about 7 mg/day.

Item 2 The protein for use according to item 1, wherein the first cycle furthermore comprises:
administering a target dose of the protein after the administration of the second dose, wherein the target dose is identical to or exceeds the second dose and is from about 9 mg/day to about 24 mg/day, preferably from 12.5 mg/day to about 24 mg/day.

Item 1a A protein comprising a first domain which binds to BCMA, a second domain which binds to CD3 and a third domain which extends the half-life of the protein, for use in the treatment or amelioration of a BCMA positive neoplasm, wherein the protein is administered in a first cycle comprising:
administering a target dose of the protein of from 12.5 mg/day to about 24 mg/day.

Item 2a The protein for use according to item 1a, wherein the first cycle comprises:
administering a first dose of the protein on day 1,
administering a second dose of the protein on a day after day 1 and before day 8, wherein the second dose exceeds the first dose and is at least about 7 mg/day, and
administering the target dose of the protein after the administration of the second dose, wherein the target dose is identical to or exceeds the second dose.

Item 2b The protein for use according to item 1a, wherein the first cycle further comprises:
administering a first dose of the protein on day 1.
administering a second dose of the protein on a day after day 1 and before day 8, wherein the second dose exceeds the first dose, and
optionally administering a third dose of the protein on a day after the day of administration of the second dose and before the day of administration of the target dose, wherein the third dose exceeds the second dose.

Item 3 The protein for use according to any one of the preceding items, wherein the second dose is administered on day 3 or day 4, preferably on day 3.

Item 4 The protein for use according to any one of the preceding items, wherein the second dose is from about 4 mg/day to about 12.5 mg/day, from about 4.5 mg/day to about 12 mg/day, from about 4 mg/day to about 10 mg/day, from about 4.5 mg/day to about 9 mg/day, from about 4 mg/day to about 8 mg/day, from about 4 mg/day to about 7 mg/day, from about 4 mg/day to about 6.5 mg/day, from about 4.5 mg/day to about 6 mg/day, from about 7 mg/day to about 18 mg/day, from about 7.5 mg/day to about 15 mg/day, from about 8 mg/day to about 12 mg/day, from about 8.5 mg/day to about 10 mg/day or from about 9 mg/day to about 9.5 mg/day.

Item 5 The protein for use according to any one of the preceding items, wherein the second dose is from about 8 mg/day to about 10 mg/day, preferably about 9 mg/day, and is administered on day 3.

Item 5a The protein for use according to any one of the preceding items, wherein the second dose is from about 4 mg/day to about 10 mg/day, preferably from about 4 mg/day to about 7 mg/day, and is administered on day 3.

Item 6 The protein for use according to any one of the preceding items, wherein the first dose is at least about 800 μg/day or is about 800 μg/day.

Item 7 The protein for use according to any one of the preceding items, wherein the first dose is from about 800 μg/day to about 1200 μg/day, from about 800 μg/day to about 1100 μg/day, from about 800 μg/day to about 1000 μg/day or from about 800 μg/day to about 900 μg/day.

Item 7a The protein for use according to any one of the preceding items, wherein the third dose is from about 7 mg/day to about 12 mg/day, from about 8 mg/day to about 11 mg/day, from about 8 mg/day to about 10 mg/day or about 9 mg/day.

Item 7b The protein for use according to any one of the preceding items, wherein the third dose is administered on day 5, day 6 or day 7.

Item 7c The protein for use according to any one of the preceding items, wherein the third dose is from about 8 mg/day to about 10 mg/day and is administered on day 5.

Item 8 The protein for use according to any one of items 2 to 7, wherein the target dose is administered on a day from day 6 to day 10, preferably on a day from day 7 to day 9, more preferably on day 8.

Item 9 The protein for use according to any one of items 2 to 8, wherein the target dose is administered on day 15 (+/− one or two days) and day 22 (+/− one or two days). (Once the target dose is reached on a day from day 6 to day 10, it is preferably administered once every 7 days.)

Item 10 The protein for use according to any one of the preceding items, wherein the target dose is from about 14 mg/day to about 22 mg/day, from about 15 mg/day to about 21 mg/day, from about 16 mg/day to about 20 mg/day, from about 17 mg/day to about 19 mg/day, or about 18 mg/day.

Item 10a The protein for use according to any one of the preceding items, wherein the target dose is from about 16 mg/day to about 20 mg/day and is administered on day 8.

Item 11 The protein for use according to any one of the preceding items, wherein the protein is administered in a second cycle and optionally in further subsequent cycles at the target dose.

Item 12 The protein for use according to item 11, wherein the second cycle and optionally the further subsequent cycles comprise:
administering the protein at the target dose on day 1, day 8 (+/− one or two days), day 15 (+/− one or two days) and day 22 (+/− one or two days).

Preferably, the second cycle and optionally the further subsequent cycles comprise administering the protein at the target dose once every 7 days.

Item 13 The protein for use according to any one of the preceding items, wherein one cycle (i.e. the first cycle, the second cycle and/or any subsequent cycle) has about 25 to about 30 days, about 26 to about 29 days, about 27 to about 29 days, or about 28 days.

Item 13a The protein for use according to any one of the preceding items, wherein the protein is administered in 2, 3, 4, 5, 6, 7, 8, 9, 10, 11, 12, 13, 14, 15, 16, 17, 18, 19, 20, 21, 22, 23, 24, 25 or more cycles.

Item 14 The protein for use according to any one of the preceding items, wherein the first cycle comprises:
administering a first dose of the protein of about 800 μg/day to about 1000 μg/day, preferably about 800 μg/day,
administering a second dose of the protein of about 8 mg/day to about 10 mg/day, preferably about 9 mg/day, and
administering a target dose of the protein of about 12 mg/day to about 20 mg/day, preferably about 16 mg/day to about 20 mg/day, such as 18 mg/day.

Item 14a The protein for use according to any one of the preceding items, wherein the first cycle comprises:
administering a first dose of the protein of about 800 μg/day to about 1000 μg/day, preferably about 800 μg/day,
administering a second dose of the protein of about 4 mg/day to about 10 mg/day, preferably about 4 mg/day to about 7 mg/day, or 4.5 mg/day to 6 mg/day,
optionally administering a third dose of the protein of about 8 mg/day to about 10 mg/day, preferably about 9 mg/day, wherein the third dose exceeds the second dose, and
administering a target dose of the protein of 14 mg/day to about 22 mg/day, preferably about 16 mg/day to about 20 mg/day, such as 18 mg/day.

Item 15 The protein for use according to item 14 or 14a, wherein the first dose is administered on day 1, the second dose is administered on day 3 or day 4, preferably on day 3, the optional third dose is administered on day 5 or day 6, preferably on day 5, and the target dose is administered on day 8 (+/− one day), day 15 (+/− one day) and day 22 (+/− one day), preferably on day 8, day 15 and day 22.

Item 15a The protein for use according to any one of the preceding items, wherein the first cycle comprises or consists of three to six or of four to six individual administrations, preferably of four or five or six individual administrations of the protein.

Item 15b The protein for use according to any one of the preceding items, wherein the first dose of 800 μg/day is administered on day 1, the second dose of 6 mg/day is administered on day 3, and the target dose of 18 mg/day is administered on day 8, day 15 and day 22.

Item 15c The protein for use according to any one of the preceding items, wherein the first dose of 800 μg/day is administered on day 1, the second dose of 4.5 mg/day is administered on day 3, the third dose of 9 mg/day is administered on day 5, and the target dose of 18 mg/day is administered on day 8, day 15 and day 22.

Item 16 The protein for use according to any one of the preceding items, wherein the protein is administered intravenously, preferably via intravenous bolus injection, bolus infusion or short-term intravenous infusion.

Item 17 The protein for use according to any one of the preceding items, wherein the BCMA positive neoplasm is selected from the group consisting of multiple myeloma, relapsed and/or refractory multiple myeloma, heavy chain multiple myeloma, light chain multiple myeloma, extramedullary myeloma, plasmacytoma, plasma cell leukemia, Waldenström's macroglobulinemia, and smoldering myeloma.

Item 17a The protein for use according to item 17, wherein the BCMA positive neoplasm is multiple myeloma.

Item 17b The protein for use according to item 17a, wherein the multiple myeloma is selected from the group consisting of relapsed and/or refractory multiple myeloma, heavy chain multiple myeloma, light chain multiple myeloma, extramedullary multiple myeloma, and smoldering multiple myeloma.

Item 18 The protein for use according to any one of the preceding items, wherein
a) the protein is a single chain protein (or “single chain polypeptide”, i.e. it consists of one polypeptide chain) or consists of two, three or four polypeptide chains,
b) the first domain comprises an immunoglobulin heavy chain variable region (VH1) and an immunoglobulin light chain variable region (VL1),
c) the second domain comprises an immunoglobulin heavy chain variable region (VH2) and an immunoglobulin light chain variable region (VL2), and/or
d) the third domain comprises one or two immunoglobulin hinge regions, one or two CH2 domains and one or two CH3 domains, preferably two immunoglobulin hinge regions, two CH2 domains and two CH3 domains.

Item 18a The protein for use according to any one of the preceding items, wherein
a) the first domain comprises an immunoglobulin heavy chain variable region (VH1) and an immunoglobulin light chain variable region (VL1),
b) the second domain comprises an immunoglobulin heavy chain variable region (VH2) and an immunoglobulin light chain variable region (VL2), and
c) the third domain comprises two immunoglobulin hinge regions, two CH2 domains and two CH3 domains.

Item 18b The protein for use according to any one of the preceding items, wherein
a) the protein is a single chain protein,
b) the first domain is in the format of an scFv,
c) the second domain is in the format of an scFv,
d) the first and the second domain are connected via a linker, preferably a peptide linker, more preferably a glycine/serine linker, and/or
e) the third domain comprises two Fc monomers, each monomer comprising an immunoglobulin hinge region, a CH2 domain and a CH3 domain, wherein the monomers are preferably fused to each other via a peptide linker.

Item 19 The protein for use according to any one of the preceding items, wherein the protein competes for binding to BCMA with or binds to the same epitope of BCMA as:
a) an antibody or protein comprising a domain which binds to BCMA on the surface of a target cell, wherein said domain comprises a VH region comprising CDR-H1 as depicted in SEQ ID NO: 171, CDR-H2 as depicted in SEQ ID NO: 172, and CDR-H3 as depicted in SEQ ID NO: 173, and a VL region comprising CDR-L1 as depicted in SEQ ID NO: 174, CDR-L2 as depicted in SEQ ID NO: 175, and CDR-L3 as depicted in SEQ ID NO: 176;
b) an antibody or protein comprising a domain which binds to BCMA on the surface of a target cell, wherein said domain comprises a VH region as depicted in SEQ ID NO: 177, and a VL region as depicted in SEQ ID NO: 178;
c) a protein comprising a domain which binds to BCMA on the surface of a target cell, wherein said domain comprises the amino acid sequence as depicted in SEQ ID NO: 179; or
d) a protein having the amino acid sequence as depicted in SEQ ID NO: 661.

Item 20 The protein for use according to any one of the preceding items, wherein the protein competes for binding to CD3 with or binds to the same epitope of CD3 as:
a) an antibody or protein comprising a domain which binds to CD3 on the surface of a T cell, wherein said domain comprises a VH region comprising CDR-H1 as depicted in SEQ ID NO: 636, CDR-H2 as depicted in SEQ ID NO: 637, and CDR-H3 as depicted in SEQ ID NO: 638, and a VL region comprising CDR-L1 as depicted in SEQ ID NO: 633, CDR-L2 as depicted in SEQ ID NO: 634, CDR-L3 as depicted in SEQ ID NO: 635;
b) an antibody or protein comprising a domain which binds to CD3 on the surface of a T cell, wherein said domain comprises a VH region as depicted in SEQ ID NO: 639, and a VL region as depicted in SEQ ID NO: 641;
c) a protein comprising a domain which binds to CD3 on the surface of a T cell, wherein said domain comprises the amino acid sequence as depicted in SEQ ID NO: 642; or
d) a protein having the amino acid sequence as depicted in SEQ ID NO: 661.

Item 21 The protein for use according to any one of the preceding items, wherein the first domain which binds to BCMA comprises a VH region comprising CDR-H1, CDR-H2 and CDR-H3 and a VL region comprising CDR-L1, CDR-L2 and CDR-L3 selected from:
(1) CDR-H1 as depicted in SEQ ID NO: 1. CDR-H2 as depicted in SEQ ID NO: 2, CDR-H3 as depicted in SEQ ID NO: 3, CDR-L1 as depicted in SEQ ID NO: 4, CDR-L2 as depicted in SEQ ID NO: 5, and CDR-L3 as depicted in SEQ ID NO: 6;
(2) CDR-H1 as depicted in SEQ ID NO: 11, CDR-H2 as depicted in SEQ ID NO: 12, CDR-H3 as depicted in SEQ ID NO: 13, CDR-L1 as depicted in SEQ ID NO: 14, CDR-L2 as depicted in SEQ ID NO: 15, and CDR-L3 as depicted in SEQ ID NO: 16;
(3) CDR-H1 as depicted in SEQ ID NO: 21, CDR-H2 as depicted in SEQ ID NO: 22, CDR-H3 as depicted in SEQ ID NO: 23, CDR-L1 as depicted in SEQ ID NO: 24, CDR-L2 as depicted in SEQ ID NO: 25, and CDR-L3 as depicted in SEQ ID NO: 26;
(4) CDR-H1 as depicted in SEQ ID NO: 31. CDR-H2 as depicted in SEQ ID NO: 32, CDR-H3 as depicted in SEQ ID NO: 33, CDR-L1 as depicted in SEQ ID NO: 34, CDR-L2 as depicted in SEQ ID NO: 35, and CDR-L3 as depicted in SEQ ID NO: 36;
(5) CDR-H1 as depicted in SEQ ID NO: 41, CDR-H2 as depicted in SEQ ID NO: 42, CDR-H3 as depicted in SEQ ID NO: 43, CDR-L1 as depicted in SEQ ID NO: 44, CDR-L2 as depicted in SEQ ID NO: 45, and CDR-L3 as depicted in SEQ ID NO: 46;
(6) CDR-H1 as depicted in SEQ ID NO: 51, CDR-H2 as depicted in SEQ ID NO: 52, CDR-H3 as depicted in SEQ ID NO: 53, CDR-L1 as depicted in SEQ ID NO: 54. CDR-L2 as depicted in SEQ ID NO: 55, and CDR-L3 as depicted in SEQ ID NO: 56;
(7) CDR-H1 as depicted in SEQ ID NO: 61, CDR-H2 as depicted in SEQ ID NO: 62, CDR-H3 as depicted in SEQ ID NO: 63, CDR-L1 as depicted in SEQ ID NO: 64, CDR-L2 as depicted in SEQ ID NO: 65, and CDR-L3 as depicted in SEQ ID NO: 66;
(8) CDR-H1 as depicted in SEQ ID NO: 71, CDR-H2 as depicted in SEQ ID NO: 72, CDR-H3 as depicted in SEQ ID NO: 73. CDR-L1 as depicted in SEQ ID NO: 74, CDR-L2 as depicted in SEQ ID NO: 75, and CDR-L3 as depicted in SEQ ID NO: 76;
(9) CDR-H1 as depicted in SEQ ID NO: 81, CDR-H2 as depicted in SEQ ID NO: 82, CDR-H3 as depicted in SEQ ID NO: 83, CDR-L1 as depicted in SEQ ID NO: 84, CDR-L2 as depicted in SEQ ID NO: 85, and CDR-L3 as depicted in SEQ ID NO: 86;
(10) CDR-H1 as depicted in SEQ ID NO: 91, CDR-H2 as depicted in SEQ ID NO: 92, CDR-H3 as depicted in SEQ ID NO: 93, CDR-L1 as depicted in SEQ ID NO: 94, CDR-L2 as depicted in SEQ ID NO: 95, and CDR-L3 as depicted in SEQ ID NO: 96;
(11) CDR-H1 as depicted in SEQ ID NO: 101, CDR-H2 as depicted in SEQ ID NO: 102, CDR-H3 as depicted in SEQ ID NO: 103, CDR-L1 as depicted in SEQ ID NO: 104. CDR-L2 as depicted in SEQ ID NO: 105, and CDR-L3 as depicted in SEQ ID NO: 106;
(12) CDR-H1 as depicted in SEQ ID NO: 111. CDR-H2 as depicted in SEQ ID NO: 112, CDR-H3 as depicted in SEQ ID NO: 113, CDR-L1 as depicted in SEQ ID NO: 114, CDR-L2 as depicted in SEQ ID NO: 115, and CDR-L3 as depicted in SEQ ID NO: 116;
(13) CDR-H1 as depicted in SEQ ID NO: 121, CDR-H2 as depicted in SEQ ID NO: 122, CDR-H3 as depicted in SEQ ID NO: 123, CDR-L1 as depicted in SEQ ID NO: 124, CDR-L2 as depicted in SEQ ID NO: 125, and CDR-L3 as depicted in SEQ ID NO: 126;
(14) CDR-H1 as depicted in SEQ ID NO: 131, CDR-H2 as depicted in SEQ ID NO: 132, CDR-H3 as depicted in SEQ ID NO: 133, CDR-L1 as depicted in SEQ ID NO: 134, CDR-L2 as depicted in SEQ ID NO: 135, and CDR-L3 as depicted in SEQ ID NO: 136;
(15) CDR-H1 as depicted in SEQ ID NO: 141, CDR-H2 as depicted in SEQ ID NO: 142, CDR-H3 as depicted in SEQ ID NO: 143, CDR-L1 as depicted in SEQ ID NO: 144, CDR-L2 as depicted in SEQ ID NO: 145, and CDR-L3 as depicted in SEQ ID NO: 146;
(16) CDR-H1 as depicted in SEQ ID NO: 151, CDR-H2 as depicted in SEQ ID NO: 152, CDR-H3 as depicted in SEQ ID NO: 153, CDR-L1 as depicted in SEQ ID NO: 154, CDR-L2 as depicted in SEQ ID NO: 155, and CDR-L3 as depicted in SEQ ID NO: 156;
(17) CDR-H1 as depicted in SEQ ID NO: 161, CDR-H2 as depicted in SEQ ID NO: 162, CDR-H3 as depicted in SEQ ID NO: 163, CDR-L1 as depicted in SEQ ID NO: 164, CDR-L2 as depicted in SEQ ID NO: 165, and CDR-L3 as depicted in SEQ ID NO: 166;
(18) CDR-H1 as depicted in SEQ ID NO: 171, CDR-H2 as depicted in SEQ ID NO: 172, CDR-H3 as depicted in SEQ ID NO: 173, CDR-L1 as depicted in SEQ ID NO: 174, CDR-L2 as depicted in SEQ ID NO: 175, and CDR-L3 as depicted in SEQ ID NO: 176;
(19) CDR-H1 as depicted in SEQ ID NO: 181, CDR-H2 as depicted in SEQ ID NO: 182, CDR-H3 as depicted in SEQ ID NO: 183, CDR-L1 as depicted in SEQ ID NO: 184, CDR-L2 as depicted in SEQ ID NO: 185, and CDR-L3 as depicted in SEQ ID NO: 186;
(20) CDR-H1 as depicted in SEQ ID NO: 191, CDR-H2 as depicted in SEQ ID NO: 192, CDR-H3 as depicted in SEQ ID NO: 193, CDR-L1 as depicted in SEQ ID NO: 194, CDR-L2 as depicted in SEQ ID NO: 195, and CDR-L3 as depicted in SEQ ID NO: 196;
(21) CDR-H1 as depicted in SEQ ID NO: 201. CDR-H2 as depicted in SEQ ID NO: 202, CDR-H3 as depicted in SEQ ID NO: 203. CDR-L1 as depicted in SEQ ID NO: 204, CDR-L2 as depicted in SEQ ID NO: 205, and CDR-L3 as depicted in SEQ ID NO: 206;
(22) CDR-H1 as depicted in SEQ ID NO: 211, CDR-H2 as depicted in SEQ ID NO: 212, CDR-H3 as depicted in SEQ ID NO: 213, CDR-L1 as depicted in SEQ ID NO: 214, CDR-L2 as depicted in SEQ ID NO: 215, and CDR-L3 as depicted in SEQ ID NO: 216;
(23) CDR-H1 as depicted in SEQ ID NO: 221, CDR-H2 as depicted in SEQ ID NO: 222, CDR-H3 as depicted in SEQ ID NO: 223, CDR-L1 as depicted in SEQ ID NO: 224, CDR-L2 as depicted in SEQ ID NO: 225, and CDR-L3 as depicted in SEQ ID NO: 226;
(24) CDR-H1 as depicted in SEQ ID NO: 231. CDR-H2 as depicted in SEQ ID NO: 232, CDR-H3 as depicted in SEQ ID NO: 233, CDR-L1 as depicted in SEQ ID NO: 234, CDR-L2 as depicted in SEQ ID NO: 235, and CDR-L3 as depicted in SEQ ID NO: 236;
(25) CDR-H1 as depicted in SEQ ID NO: 241, CDR-H2 as depicted in SEQ ID NO: 242, CDR-H3 as depicted in SEQ ID NO: 243, CDR-L1 as depicted in SEQ ID NO: 244, CDR-L2 as depicted in SEQ ID NO: 245, and CDR-L3 as depicted in SEQ ID NO: 246;
(26) CDR-H1 as depicted in SEQ ID NO: 251, CDR-H2 as depicted in SEQ ID NO: 252, CDR-H3 as depicted in SEQ ID NO: 253, CDR-L1 as depicted in SEQ ID NO: 254, CDR-L2 as depicted in SEQ ID NO: 255, and CDR-L3 as depicted in SEQ ID NO: 256;
(27) CDR-H1 as depicted in SEQ ID NO: 261, CDR-H2 as depicted in SEQ ID NO: 262, CDR-H3 as depicted in SEQ ID NO: 263, CDR-L1 as depicted in SEQ ID NO: 264, CDR-L2 as depicted in SEQ ID NO: 265, and CDR-L3 as depicted in SEQ ID NO: 266;
(28) CDR-H1 as depicted in SEQ ID NO: 271. CDR-H2 as depicted in SEQ ID NO: 272, CDR-H3 as depicted in SEQ ID NO: 273, CDR-L1 as depicted in SEQ ID NO: 274, CDR-L2 as depicted in SEQ ID NO: 275, and CDR-L3 as depicted in SEQ ID NO: 276;
(29) CDR-H1 as depicted in SEQ ID NO: 281, CDR-H2 as depicted in SEQ ID NO: 282, CDR-H3 as depicted in SEQ ID NO: 283, CDR-L1 as depicted in SEQ ID NO: 284, CDR-L2 as depicted in SEQ ID NO: 285, and CDR-L3 as depicted in SEQ ID NO: 286;
(30) CDR-H1 as depicted in SEQ ID NO: 291, CDR-H2 as depicted in SEQ ID NO: 292, CDR-H3 as depicted in SEQ ID NO: 293, CDR-L1 as depicted in SEQ ID NO: 294, CDR-L2 as depicted in SEQ ID NO: 295, and CDR-L3 as depicted in SEQ ID NO: 296;
(31) CDR-H1 as depicted in SEQ ID NO: 301, CDR-H2 as depicted in SEQ ID NO: 302, CDR-H3 as depicted in SEQ ID NO: 303, CDR-L1 as depicted in SEQ ID NO: 304, CDR-L2 as depicted in SEQ ID NO: 305, and CDR-L3 as depicted in SEQ ID NO: 306;
(32) CDR-H1 as depicted in SEQ ID NO: 311, CDR-H2 as depicted in SEQ ID NO: 312, CDR-H3 as depicted in SEQ ID NO: 313, CDR-L1 as depicted in SEQ ID NO: 314, CDR-L2 as depicted in SEQ ID NO: 315, and CDR-L3 as depicted in SEQ ID NO: 316;
(33) CDR-H1 as depicted in SEQ ID NO: 321. CDR-H2 as depicted in SEQ ID NO: 322, CDR-H3 as depicted in SEQ ID NO: 323, CDR-L1 as depicted in SEQ ID NO: 324, CDR-L2 as depicted in SEQ ID NO: 325, and CDR-L3 as depicted in SEQ ID NO: 326;
(34) CDR-H1 as depicted in SEQ ID NO: 331, CDR-H2 as depicted in SEQ ID NO: 332, CDR-H3 as depicted in SEQ ID NO: 333, CDR-L1 as depicted in SEQ ID NO: 334, CDR-L2 as depicted in SEQ ID NO: 335, and CDR-L3 as depicted in SEQ ID NO: 336;
(35) CDR-H1 as depicted in SEQ ID NO: 341, CDR-H2 as depicted in SEQ ID NO: 342, CDR-H3 as depicted in SEQ ID NO: 343, CDR-L1 as depicted in SEQ ID NO: 344, CDR-L2 as depicted in SEQ ID NO: 345, and CDR-L3 as depicted in SEQ ID NO: 346;
(36) CDR-H1 as depicted in SEQ ID NO: 351. CDR-H2 as depicted in SEQ ID NO: 352, CDR-H3 as depicted in SEQ ID NO: 353, CDR-L1 as depicted in SEQ ID NO: 354, CDR-L2 as depicted in SEQ ID NO: 355, and CDR-L3 as depicted in SEQ ID NO: 356;
(37) CDR-H1 as depicted in SEQ ID NO: 361, CDR-H2 as depicted in SEQ ID NO: 362, CDR-H3 as depicted in SEQ ID NO: 363. CDR-L1 as depicted in SEQ ID NO: 364, CDR-L2 as depicted in SEQ ID NO: 365, and CDR-L3 as depicted in SEQ ID NO: 366;
(38) CDR-H1 as depicted in SEQ ID NO: 371, CDR-H2 as depicted in SEQ ID NO: 372, CDR-H3 as depicted in SEQ ID NO: 373, CDR-L1 as depicted in SEQ ID NO: 374, CDR-L2 as depicted in SEQ ID NO: 375, and CDR-L3 as depicted in SEQ ID NO: 376;
(39) CDR-H1 as depicted in SEQ ID NO: 381, CDR-H2 as depicted in SEQ ID NO: 382, CDR-H3 as depicted in SEQ ID NO: 383, CDR-L1 as depicted in SEQ ID NO: 384, CDR-L2 as depicted in SEQ ID NO: 385, and CDR-L3 as depicted in SEQ ID NO: 386;
(40) CDR-H1 as depicted in SEQ ID NO: 391. CDR-H2 as depicted in SEQ ID NO: 392, CDR-H3 as depicted in SEQ ID NO: 393, CDR-L1 as depicted in SEQ ID NO: 394, CDR-L2 as depicted in SEQ ID NO: 395, and CDR-L3 as depicted in SEQ ID NO: 396;
(41) CDR-H1 as depicted in SEQ ID NO: 401, CDR-H2 as depicted in SEQ ID NO: 402, CDR-H3 as depicted in SEQ ID NO: 403, CDR-L1 as depicted in SEQ ID NO: 404, CDR-L2 as depicted in SEQ ID NO: 405, and CDR-L3 as depicted in SEQ ID NO: 406;
(42) CDR-H1 as depicted in SEQ ID NO: 411, CDR-H2 as depicted in SEQ ID NO: 412, CDR-H3 as depicted in SEQ ID NO: 413, CDR-L1 as depicted in SEQ ID NO: 414, CDR-L2 as depicted in SEQ ID NO: 415, and CDR-L3 as depicted in SEQ ID NO: 416;
(43) CDR-H1 as depicted in SEQ ID NO: 421, CDR-H2 as depicted in SEQ ID NO: 422, CDR-H3 as depicted in SEQ ID NO: 423, CDR-L1 as depicted in SEQ ID NO: 424, CDR-L2 as depicted in SEQ ID NO: 425, and CDR-L3 as depicted in SEQ ID NO: 426;
(44) CDR-H1 as depicted in SEQ ID NO: 431, CDR-H2 as depicted in SEQ ID NO: 432, CDR-H3 as depicted in SEQ ID NO: 433, CDR-L1 as depicted in SEQ ID NO: 434, CDR-L2 as depicted in SEQ ID NO: 435, and CDR-L3 as depicted in SEQ ID NO: 436;
(45) CDR-H1 as depicted in SEQ ID NO: 441. CDR-H2 as depicted in SEQ ID NO: 442, CDR-H3 as depicted in SEQ ID NO: 443, CDR-L1 as depicted in SEQ ID NO: 444, CDR-L2 as depicted in SEQ ID NO: 445, and CDR-L3 as depicted in SEQ ID NO: 446;
(46) CDR-H1 as depicted in SEQ ID NO: 451, CDR-H2 as depicted in SEQ ID NO: 452, CDR-H3 as depicted in SEQ ID NO: 453, CDR-L1 as depicted in SEQ ID NO: 454, CDR-L2 as depicted in SEQ ID NO: 455, and CDR-L3 as depicted in SEQ ID NO: 456;
(47) CDR-H1 as depicted in SEQ ID NO: 461, CDR-H2 as depicted in SEQ ID NO: 462, CDR-H3 as depicted in SEQ ID NO: 463, CDR-L1 as depicted in SEQ ID NO: 464, CDR-L2 as depicted in SEQ ID NO: 465, and CDR-L3 as depicted in SEQ ID NO: 466;
(48) CDR-H1 as depicted in SEQ ID NO: 471, CDR-H2 as depicted in SEQ ID NO: 472, CDR-H3 as depicted in SEQ ID NO: 473, CDR-L1 as depicted in SEQ ID NO: 474, CDR-L2 as depicted in SEQ ID NO: 475, and CDR-L3 as depicted in SEQ ID NO: 476;
(49) CDR-H1 as depicted in SEQ ID NO: 481, CDR-H2 as depicted in SEQ ID NO: 482, CDR-H3 as depicted in SEQ ID NO: 483, CDR-L1 as depicted in SEQ ID NO: 484, CDR-L2 as depicted in SEQ ID NO: 485, and CDR-L3 as depicted in SEQ ID NO: 486;
(50) CDR-H1 as depicted in SEQ ID NO: 491, CDR-H2 as depicted in SEQ ID NO: 492, CDR-H3 as depicted in SEQ ID NO: 493, CDR-L1 as depicted in SEQ ID NO: 494, CDR-L2 as depicted in SEQ ID NO: 495, and CDR-L3 as depicted in SEQ ID NO: 496;
(51) CDR-H1 as depicted in SEQ ID NO: 501, CDR-H2 as depicted in SEQ ID NO: 502, CDR-H3 as depicted in SEQ ID NO: 503, CDR-L1 as depicted in SEQ ID NO: 504, CDR-L2 as depicted in SEQ ID NO: 505, and CDR-L3 as depicted in SEQ ID NO: 506;
(52) CDR-H1 as depicted in SEQ ID NO: 511. CDR-H2 as depicted in SEQ ID NO: 512, CDR-H3 as depicted in SEQ ID NO: 513, CDR-L1 as depicted in SEQ ID NO: 514, CDR-L2 as depicted in SEQ ID NO: 515, and CDR-L3 as depicted in SEQ ID NO: 516; and
(53) CDR-H1 as depicted in SEQ ID NO: 521, CDR-H2 as depicted in SEQ ID NO: 522, CDR-H3 as depicted in SEQ ID NO: 523, CDR-L1 as depicted in SEQ ID NO: 524, CDR-L2 as depicted in SEQ ID NO: 525, and CDR-L3 as depicted in SEQ ID NO: 526.

Item 22 The protein for use according to any one of the preceding items, wherein the first domain which binds to BCMA comprises a VH region having an amino acid sequence selected from the group consisting of those depicted in SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 307, 317, 327, 337, 347, 357, 367, 377, 387, 397, 407, 417, 427, 437, 447, 457, 467, 477, 487, 497, 507, 517, and 527, preferably SEQ ID NO: 177.

Item 23 The protein for use according to any one of the preceding items, wherein the first domain which binds to BCMA comprises a VL region having an amino acid sequence selected from the group consisting of those depicted in SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, and 528, preferably SEQ ID NO: 178.

Item 24 The protein for use according to any one of the preceding items, wherein the first domain which binds to BCMA comprises a VH region and a VL region selected from the group consisting of:
(1) a VH region as depicted in SEQ ID NO: 7 and a VL region as depicted in SEQ ID NO: 8;
(2) a VH region as depicted in SEQ ID NO: 17 and a VL region as depicted in SEQ ID NO: 18;
(3) a VH region as depicted in SEQ ID NO: 27 and a VL region as depicted in SEQ ID NO: 28;
(4) a VH region as depicted in SEQ ID NO: 37 and a VL region as depicted in SEQ ID NO: 38;
(5) a VH region as depicted in SEQ ID NO: 47 and a VL region as depicted in SEQ ID NO: 48;
(6) a VH region as depicted in SEQ ID NO: 57 and a VL region as depicted in SEQ ID NO: 58;
(7) a VH region as depicted in SEQ ID NO: 67 and a VL region as depicted in SEQ ID NO: 68;
(8) a VH region as depicted in SEQ ID NO: 77 and a VL region as depicted in SEQ ID NO: 78;
(9) a VH region as depicted in SEQ ID NO: 87 and a VL region as depicted in SEQ ID NO: 88;
(10) a VH region as depicted in SEQ ID NO: 97 and a VL region as depicted in SEQ ID NO: 98;
(11) a VH region as depicted in SEQ ID NO: 107 and a VL region as depicted in SEQ ID NO: 108;
(12) a VH region as depicted in SEQ ID NO: 117 and a VL region as depicted in SEQ ID NO: 118;
(13) a VH region as depicted in SEQ ID NO: 127 and a VL region as depicted in SEQ ID NO: 128;
(14) a VH region as depicted in SEQ ID NO: 137 and a VL region as depicted in SEQ ID NO: 138;
(15) a VH region as depicted in SEQ ID NO: 147 and a VL region as depicted in SEQ ID NO: 148;
(16) a VH region as depicted in SEQ ID NO: 157 and a VL region as depicted in SEQ ID NO: 158;
(17) a VH region as depicted in SEQ ID NO: 167 and a VL region as depicted in SEQ ID NO: 168;
(18) a VH region as depicted in SEQ ID NO: 177 and a VL region as depicted in SEQ ID NO: 178;
(19) a VH region as depicted in SEQ ID NO: 187 and a VL region as depicted in SEQ ID NO: 188;
(20) a VH region as depicted in SEQ ID NO: 197 and a VL region as depicted in SEQ ID NO: 198;
(21) a VH region as depicted in SEQ ID NO: 207 and a VL region as depicted in SEQ ID NO: 208;
(22) a VH region as depicted in SEQ ID NO: 217 and a VL region as depicted in SEQ ID NO: 218;
(23) a VH region as depicted in SEQ ID NO: 227 and a VL region as depicted in SEQ ID NO: 228;
(24) a VH region as depicted in SEQ ID NO: 237 and a VL region as depicted in SEQ ID NO: 238;
(25) a VH region as depicted in SEQ ID NO: 247 and a VL region as depicted in SEQ ID NO: 248;
(26) a VH region as depicted in SEQ ID NO: 257 and a VL region as depicted in SEQ ID NO: 258;
(27) a VH region as depicted in SEQ ID NO: 267 and a VL region as depicted in SEQ ID NO: 268;
(28) a VH region as depicted in SEQ ID NO: 277 and a VL region as depicted in SEQ ID NO: 278;
(29) a VH region as depicted in SEQ ID NO: 287 and a VL region as depicted in SEQ ID NO: 288;
(30) a VH region as depicted in SEQ ID NO: 297 and a VL region as depicted in SEQ ID NO: 298;
(31) a VH region as depicted in SEQ ID NO: 307 and a VL region as depicted in SEQ ID NO: 308;
(32) a VH region as depicted in SEQ ID NO: 317 and a VL region as depicted in SEQ ID NO: 318;
(33) a VH region as depicted in SEQ ID NO: 327 and a VL region as depicted in SEQ ID NO: 328;
(34) a VH region as depicted in SEQ ID NO: 337 and a VL region as depicted in SEQ ID NO: 338;
(35) a VH region as depicted in SEQ ID NO: 347 and a VL region as depicted in SEQ ID NO: 348;
(36) a VH region as depicted in SEQ ID NO: 357 and a VL region as depicted in SEQ ID NO: 358;
(37) a VH region as depicted in SEQ ID NO: 367 and a VL region as depicted in SEQ ID NO: 368;
(38) a VH region as depicted in SEQ ID NO: 377 and a VL region as depicted in SEQ ID NO: 378;
(39) a VH region as depicted in SEQ ID NO: 387 and a VL region as depicted in SEQ ID NO: 388;
(40) a VH region as depicted in SEQ ID NO: 397 and a VL region as depicted in SEQ ID NO: 398;
(41) a VH region as depicted in SEQ ID NO: 407 and a VL region as depicted in SEQ ID NO: 408;
(42) a VH region as depicted in SEQ ID NO: 417 and a VL region as depicted in SEQ ID NO: 418;
(43) a VH region as depicted in SEQ ID NO: 427 and a VL region as depicted in SEQ ID NO: 428;
(44) a VH region as depicted in SEQ ID NO: 437 and a VL region as depicted in SEQ ID NO: 438;
(45) a VH region as depicted in SEQ ID NO: 447 and a VL region as depicted in SEQ ID NO: 448;
(46) a VH region as depicted in SEQ ID NO: 457 and a VL region as depicted in SEQ ID NO: 458;
(47) a VH region as depicted in SEQ ID NO: 467 and a VL region as depicted in SEQ ID NO: 468;
(48) a VH region as depicted in SEQ ID NO: 477 and a VL region as depicted in SEQ ID NO: 478;
(49) a VH region as depicted in SEQ ID NO: 487 and a VL region as depicted in SEQ ID NO: 488;
(50) a VH region as depicted in SEQ ID NO: 497 and a VL region as depicted in SEQ ID NO: 498;
(51) a VH region as depicted in SEQ ID NO: 507 and a VL region as depicted in SEQ ID NO: 508;
(52) a VH region as depicted in SEQ ID NO: 517 and a VL region as depicted in SEQ ID NO: 518; and
(53) a VH region as depicted in SEQ ID NO: 527 and a VL region as depicted in SEQ ID NO: 528.

Item 25 The protein for use according to any one of the preceding items, wherein the first domain which binds to BCMA comprises or consists of a polypeptide having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529, preferably SEQ ID NO: 179.

Item 26 The protein for use according to any one of the preceding items, wherein the second domain which binds to CD3 comprises a VL region comprising CDR-L1. CDR-L2 and CDR-L3 selected from the group consisting of:
(a) CDR-L1 as depicted in SEQ ID NO: 542, CDR-L2 as depicted in SEQ ID NO: 543, and CDR-L3 as depicted in SEQ ID NO: 544;
(b) CDR-L1 as depicted in SEQ ID NO: 599, CDR-L2 as depicted in SEQ ID NO: 600, and CDR-L3 as depicted in SEQ ID NO: 601; and
(c) CDR-L1 as depicted in SEQ ID NO: 621, CDR-L2 as depicted in SEQ ID NO: 622, and CDR-L3 as depicted in SEQ ID NO: 623.

Item 27 The protein for use according to any one of the preceding items, wherein the second domain which binds to CD3 comprises a VH region comprising CDR-H1, CDR-H2 and CDR-H3 selected from the group consisting of:
(a) CDR-H1 as depicted in SEQ ID NO: 534, CDR-H2 as depicted in SEQ ID NO: 535, and CDR-H3 as depicted in SEQ ID NO: 536;
(b) CDR-H1 as depicted in SEQ ID NO: 545, CDR-H2 as depicted in SEQ ID NO: 546, and CDR-H3 as depicted in SEQ ID NO: 547;
(c) CDR-H1 as depicted in SEQ ID NO: 557, CDR-H2 as depicted in SEQ ID NO: 558, and CDR-H3 as depicted in SEQ ID NO: 559;
(d) CDR-H1 as depicted in SEQ ID NO: 568, CDR-H2 as depicted in SEQ ID NO: 569, and CDR-H3 as depicted in SEQ ID NO: 570;
(c) CDR-H1 as depicted in SEQ ID NO: 579. CDR-H2 as depicted in SEQ ID NO: 580, and CDR-H3 as depicted in SEQ ID NO: 581;
(f) CDR-H1 as depicted in SEQ ID NO: 591, CDR-H2 as depicted in SEQ ID NO: 592, and CDR-H3 as depicted in SEQ ID NO: 593;
(g) CDR-H1 as depicted in SEQ ID NO: 602, CDR-H2 as depicted in SEQ ID NO: 603, and CDR-H3 as depicted in SEQ ID NO: 604;
(h) CDR-H1 as depicted in SEQ ID NO: 613, CDR-H2 as depicted in SEQ ID NO: 614, and CDR-H3 as depicted in SEQ ID NO: 615;
(i) CDR-H1 as depicted in SEQ ID NO: 624, CDR-H2 as depicted in SEQ ID NO: 625, and CDR-H3 as depicted in SEQ ID NO: 626; and
(j) CDR-H1 as depicted in SEQ ID NO: 636, CDR-H2 as depicted in SEQ ID NO: 637, and CDR-H3 as depicted in SEQ ID NO: 638.

Item 28 The protein for use according to any one of the preceding items, wherein the second domain which binds to CD3 comprises a VL region comprising CDR-L1. CDR-L2 and CDR-L3 and a VH region comprising CDR-H1, CDR-H2 and CDR-H3 selected from the group consisting of:
(a) CDR-L1 as depicted in SEQ ID NO: 531, CDR-L2 as depicted in SEQ ID NO: 532, CDR-L3 as depicted in SEQ ID NO: 533, CDR-H1 as depicted in SEQ ID NO: 534, CDR-H2 as depicted in SEQ ID NO: 535, and CDR-H3 as depicted in SEQ ID NO: 536;
(b) CDR-L1 as depicted in SEQ ID NO: 542, CDR-L2 as depicted in SEQ ID NO: 543, CDR-L3 as depicted in SEQ ID NO: 544, CDR-H1 as depicted in SEQ ID NO: 545, CDR-H2 as depicted in SEQ ID NO: 546, and CDR-H3 as depicted in SEQ ID NO: 547;
(c) CDR-L1 as depicted in SEQ ID NO: 554, CDR-L2 as depicted in SEQ ID NO: 555, CDR-L3 as depicted in SEQ ID NO: 556, CDR-H1 as depicted in SEQ ID NO: 557, CDR-H2 as depicted in SEQ ID NO: 558, and CDR-H3 as depicted in SEQ ID NO: 559;
(d) CDR-L1 as depicted in SEQ ID NO: 565, CDR-L2 as depicted in SEQ ID NO: 566, CDR-L3 as depicted in SEQ ID NO: 567, CDR-H1 as depicted in SEQ ID NO: 568, CDR-H2 as depicted in SEQ ID NO: 569, and CDR-H3 as depicted in SEQ ID NO: 570;
(c) CDR-L1 as depicted in SEQ ID NO: 576, CDR-L2 as depicted in SEQ ID NO: 577, CDR-L3 as depicted in SEQ ID NO: 578, CDR-H1 as depicted in SEQ ID NO: 579, CDR-H2 as depicted in SEQ ID NO: 580, and CDR-H3 as depicted in SEQ ID NO: 581;
(f) CDR-L1 as depicted in SEQ ID NO: 588, CDR-L2 as depicted in SEQ ID NO: 589, CDR-L3 as depicted in SEQ ID NO: 590, CDR-H1 as depicted in SEQ ID NO: 591, CDR-H2 as depicted in SEQ ID NO: 592, and CDR-H3 as depicted in SEQ ID NO: 593;
(g) CDR-L1 as depicted in SEQ ID NO: 599, CDR-L2 as depicted in SEQ ID NO: 600, CDR-L3 as depicted in SEQ ID NO: 601, CDR-H1 as depicted in SEQ ID NO: 602, CDR-H2 as depicted in SEQ ID NO: 603, and CDR-H3 as depicted in SEQ ID NO: 604;
(h) CDR-L1 as depicted in SEQ ID NO: 610, CDR-L2 as depicted in SEQ ID NO: 611, CDR-L3 as depicted in SEQ ID NO: 612, CDR-H1 as depicted in SEQ ID NO: 613, CDR-H2 as depicted in SEQ ID NO: 614, and CDR-H3 as depicted in SEQ ID NO: 615;
(i) CDR-L1 as depicted in SEQ ID NO: 621. CDR-L2 as depicted in SEQ ID NO: 622, CDR-L3 as depicted in SEQ ID NO: 623, CDR-H1 as depicted in SEQ ID NO: 624, CDR-H2 as depicted in SEQ ID NO: 625, and CDR-H3 as depicted in SEQ ID NO: 626; and
(j) CDR-L1 as depicted in SEQ ID NO: 633, CDR-L2 as depicted in SEQ ID NO: 634, CDR-L3 as depicted in SEQ ID NO: 635, CDR-H1 as depicted in SEQ ID NO: 636, CDR-H2 as depicted in SEQ ID NO: 637, and CDR-H3 as depicted in SEQ ID NO: 638.

Item 29 The protein for use according to any one of the preceding items, wherein the second domain which binds to CD3 comprises a VL region having an amino acid sequence selected from the group consisting of those depicted in SEQ ID NO: 550, SEQ ID NO: 551, SEQ ID NO: 584, SEQ ID NO: 585, SEQ ID NO: 629 and SEQ ID NO: 630, preferably SEQ ID NO: 629.

Item 30 The protein for use according to any one of the preceding items, wherein the second domain which binds to CD3 comprises a VH region having an amino acid sequence selected from the group consisting of those depicted in SEQ ID NO: 537, SEQ ID NO: 538, SEQ ID NO: 548, SEQ ID NO: 549, SEQ ID NO: 560, SEQ ID NO: 561, SEQ ID NO: 571, SEQ ID NO: 572, SEQ ID NO: 582, SEQ ID NO: 583, SEQ ID NO: 594, SEQ ID NO: 595, SEQ ID NO: 605, SEQ ID NO: 606, SEQ ID NO: 616, SEQ ID NO: 617, SEQ ID NO: 627, SEQ ID NO: 628, SEQ ID NO: 639, SEQ ID NO: 640, and SEQ ID NO: 644, preferably SEQ ID NO: 639.

Item 31 The protein for use according to any one of the preceding items, wherein the second domain which binds to CD3 comprises a VL region and a VH region selected from the group consisting of:
(a) a VL region as depicted in SEQ ID NO: 539 or 521 and a VH region as depicted in SEQ ID NO: 537 or 538;
(b) a VL region as depicted in SEQ ID NO: 550 or 521 and a VH region as depicted in SEQ ID NO: 548 or 549;
(c) a VL region as depicted in SEQ ID NO: 562 or 521 and a VH region as depicted in SEQ ID NO: 560 or 561;
(d) a VL region as depicted in SEQ ID NO: 573 or 521 and a VH region as depicted in SEQ ID NO: 571 or 572;
(e) a VL region as depicted in SEQ ID NO: 584 or 585 and a VH region as depicted in SEQ ID NO: 582 or 583;
(f) a VL region as depicted in SEQ ID NO: 596 or 521 and a VH region as depicted in SEQ ID NO: 594 or 595;
(g) a VL region as depicted in SEQ ID NO: 607 or 585 and a VH region as depicted in SEQ ID NO: 605 or 606;
(h) a VL region as depicted in SEQ ID NO: 618 or 521 and a VH region as depicted in SEQ ID NO: 616 or 617;
(i) a VL region as depicted in SEQ ID NO: 629 or 630 and a VH region as depicted in SEQ ID NO: 627 or 628;
(j) a VL region as depicted in SEQ ID NO: 641 or 630 and a VH region as depicted in SEQ ID NO: 639 or 640; and
(k) a VL region as depicted in SEQ ID NO: 645 and a VH region as depicted in SEQ ID NO: 644.

Item 32 The protein for use according to any one of the preceding items, wherein the second domain which binds to CD3 comprises or consists of a polypeptide having an amino acid sequence selected from the group consisting of those depicted in SEQ ID NOs: 540, 541, 552, 553, 563, 564, 574, 575, 586, 587, 597, 598, 608, 609, 619, 620, 631, 632, 642, 643, and 646, preferably SEQ ID NO: 642.

Item 33 The protein for use according to any one of the preceding items, wherein the protein comprises a polypeptide having an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530.

Item 34 The protein for use according to any one of the preceding items, comprising or consisting of, in an N- to C-terminal order:
(a) the first domain;
(b) a peptide linker having an amino acid sequence selected from the group consisting of SEQ ID NOs: 687, 693 and 694;
(c) the second domain;
(d) a peptide linker having an amino acid sequence selected from the group consisting of SEQ ID NO: 686, 687, 688, 689, 690, 691, 692, 693 or 694;
(e) the third domain comprising
   (i) a first polypeptide monomer (preferably comprising a hinge, a CH2 domain and a CH3 domain);
   (ii) a peptide linker having an amino acid sequence selected from the group consisting of SEQ ID NOs: 695, 696, 697, 698 or 699; and
   (iii) a second polypeptide monomer (preferably comprising a hinge, a CH2 domain and a CH3 domain).

Item 35 The protein for use according to any one of the preceding items, comprising or consisting of, in an N- to C-terminal order:
a) the first domain having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529; wherein the peptide linker comprised within those sequences and having SEQ ID NO: 694 can be replaced by any one of SEQ ID NOs: 686-693 and 695-699;
b) a peptide linker having an amino acid sequence selected from the group consisting of SEQ ID NOs: 687, 693 and 694;
c) the second domain having an amino acid sequence selected from the group consisting of SEQ ID NOs: 540, 541, 552, 553, 563, 564, 574, 575, 586, 587, 597, 598, 608, 609, 619, 620, 631, 632, 642, 643, and 646; wherein the peptide linker comprised within those sequences and having SEQ ID NO: 694 can be replaced by any one of SEQ ID NOs: 686-693 and 695-699;
d) a peptide linker having an amino acid sequence selected from the group consisting of SEQ ID NOs: 686, 687, 688, 689, 690, 691, 692, 693, and 694; and
e) the third domain having an amino acid sequence selected from the group consisting of SEQ ID NOs: 700-707.

Item 36 The protein for use according to any one of the preceding items, comprising or consisting of, in an N- to C-terminal order:
(a) a polypeptide having an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530;
(b) a linker having an amino acid sequence as depicted in SEQ ID NO: 686; and
(c) a polypeptide having an amino acid sequence as depicted in SEQ ID NO: 700.

Item 36a The protein for use according to any one of the preceding items, wherein the protein comprises or consists of a polypeptide having the amino acid sequence as depicted in SEQ ID NO: 661.

Item 37 The protein for use according to any one of the preceding items, wherein the protein has a molecular weight of about 75 to about 200 kDa, about 80 to about 175 kDa, about 85 to about 150 kDa, about 90 to about 130 kDa, about 95 to about 120 kDa, and preferably about 100 to about 115 kDa or about 105 to about 110 kDa.

Item 38 The protein for use according to any one of the preceding items, wherein the protein has a half-life or an elimination half-life (T_{1/2}) in humans of about 3 days to about 14 days, about 4 days to about 12 days, about 3 or 4 days to about 10 days, about 3 or 4 days to about 8 days, or about 5 to about 7 days, or about 6 days.

### BRIEF DESCRIPTION OF THE DRAWINGS

FIG. 1 shows a week 1 step dosing optimization protocol. The “Step Dose” as designated in FIG. 1 corresponds to the first dose, the “Step Dose 2” corresponds to the second dose.

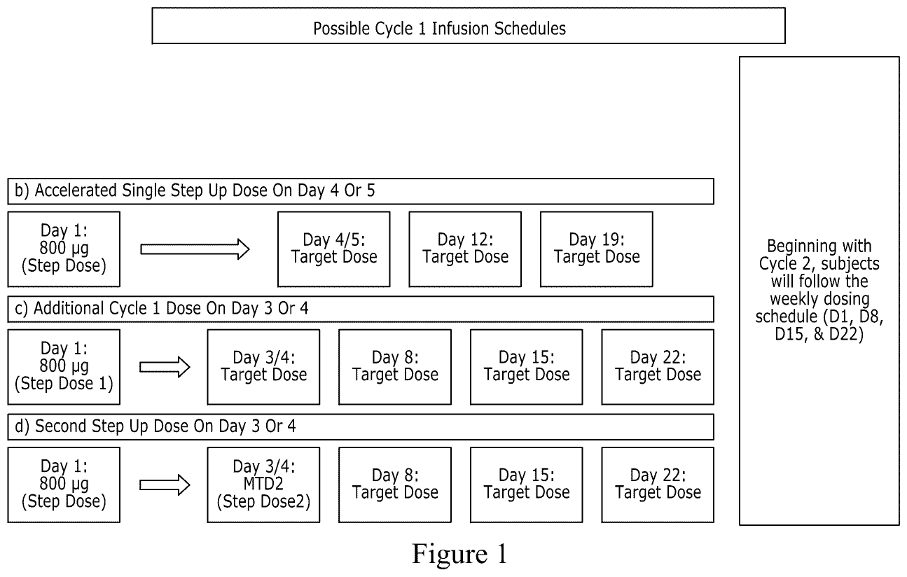

FIG. 2 shows the overall response rates of the AMG 701 clinical trial (data cut-off Q3-2020).

FIG. 3 shows an updated version of the AMG 701 clinical trial cohorts (data cut-off September 2021) with cohorts 15B and 15C ongoing. ORR=overall response rate. CRS=cytokine release syndrome.

### DETAILED DESCRIPTION OF THE INVENTION

It is an object of the present invention to provide an administration scheme for a half-life extended anti-BCMA x anti-CD3 binding protein which provides for a favorable safety and tolerability profile, while resulting in a positive efficacy signal in patients with BCMA positive neoplasms.

The present invention therefore relates to a protein comprising a first domain which binds to BCMA, a second domain which binds to CD3 and a third domain which extends the half-life of the protein, for use in the treatment or amelioration of a BCMA positive neoplasm, wherein the protein is administered in a first cycle comprising:
administering a first dose of the protein on day 1, and
administering a second dose of the protein on a day after day 1 and before day 8, wherein the second dose exceeds the first dose and is at least about 7 mg/day.

It is also envisaged that the first cycle furthermore comprises:
administering a target dose of the protein after the administration of the second dose, wherein the target dose is identical to or exceeds the second dose and is from about 9 mg/day to about 24 mg/day, preferably from 12.5 mg/day to about 24 mg/day.

The present invention also relates to a protein comprising a first domain which binds to BCMA, a second domain which binds to CD3 and a third domain which extends the half-life of the protein, for use in the treatment or amelioration of a BCMA positive neoplasm, wherein the protein is administered in a first cycle comprising:
administering a target dose of the protein of from 12.5 mg/day to about 24 mg/day.

It is also envisaged that the first cycle comprises:
administering a first dose of the protein on day 1,
administering a second dose of the protein on a day after day 1 and before day 8, wherein the second dose exceeds the first dose and is at least about 7 mg/day, and
administering the target dose of the protein after the administration of the second dose, wherein the target dose is identical to or exceeds the second dose.

The present invention also relates to a protein comprising a first domain which binds to BCMA, a second domain which binds to CD3 and a third domain which extends the half-life of the protein, for use in the treatment or amelioration of a BCMA positive neoplasm, wherein the protein is administered in a first cycle comprising:
administering a target dose of the protein of from 12.5 mg/day to about 24 mg/day.

It is also envisaged that the first cycle further comprises (in addition to the target dose of from 12.5 mg/day to about 24 mg/day):
administering a first dose of the protein on day 1.
administering a second dose of the protein on a day after day 1 and before day 8, wherein the second dose exceeds the first dose, and
optionally administering a third dose of the protein on a day after the day of administration of the second dose and before the day of administration of the target dose, wherein the third dose exceeds the second dose.

It is envisaged that the target dose is administered after the first, the second and the (optional) third dose. The target dose may e.g. by administered on a day between day 6 and day 10, such as on day 6, on day 7, on day 8, on day 9 or on day 10. It is envisaged that the target dose exceeds the first dose, the second dose as well as the (optional) third dose.

Moreover, the invention relates to a method for the treatment or amelioration of a BCMA positive neoplasm, comprising administering a protein comprising a first domain which binds to BCMA, a second domain which binds to CD3 and a third domain which extends the half-life of the protein, to a subject in need thereof, wherein the protein is administered as described above. The invention also relates to a method for administering a protein to a patient diagnosed with a BCMA positive neoplasm, wherein the protein comprises a first domain which binds to BCMA, a second domain which binds to CD3 and a third domain which extends the half-life of the protein, and wherein the protein is administered as described above. The invention further relates to the use of a protein a first domain which binds to BCMA, a second domain which binds to CD3 and a third domain which extends the half-life of the protein, for the manufacture of a medicament for the treatment of a BCMA positive neoplasm, wherein the protein is administered as described above. The invention also relates to the use of a protein comprising a first domain which binds to BCMA, a second domain which binds to CD3 and a third domain which extends the half-life of the protein, for the treatment or amelioration of a BCMA positive neoplasm, wherein the protein is administered as described above.

A “neoplasm” is an abnormal growth of tissue, usually but not always forming a mass. When also forming a mass, it is commonly referred to as a “tumor”. In brain tumors, the uncontrolled division of cells means that the mass of a neoplasm increases in size, and in a confined space such as the intracranial cavity this quickly becomes problematic because the mass invades the space of the brain pushing it aside, leading to compression of the brain tissue and increased intracranial pressure and destruction of parenchyma. According to the invention, the term “neoplasm” or “tumor” also refers to a condition that would benefit from treatment with the protein as described herein. This includes chronic and acute disorders or diseases, including those pathological conditions that predispose a mammal to the condition (neoplasm or tumor) in question.

Neoplasms or tumors can be benign, potentially malignant (pre-cancerous), or malignant (cancerous). Malignant neoplasms/tumors are commonly called cancer. They usually invade and destroy the surrounding tissue and may form metastases, i.e., they spread to other parts, tissues or organs of the body. A “primary tumor” is a tumor growing at the anatomical site where tumor progression began and proceeded to yield a cancerous mass. For example, a brain tumor occurs when abnormal cells form within the brain. Most cancers develop at their primary site but then go on to form metastases or spread to other parts (e.g. tissues and organs) of the body. These further tumors are secondary tumors. Most cancers continue to be called after their primary site, even after they have spread to other parts of the body.

Lymphomas and leukemias are hematopoietic or lymphoid neoplasms. Multiple Myeloma (MM), also known as plasma cell myeloma, is a cancer of plasma cells, a type of white blood cells that normally produce antibodies. For the purposes of the present invention, lymphomas and leukemias as well as MM are also encompassed by the terms “tumor”, “cancer” or “neoplasm”. Lymphoma is a group of blood cancers that develop from lymphocytes (a type of white blood cell). Leukemia is a group of cancers that usually begin in the bone marrow and result in high numbers of abnormal white blood cells. These white blood cells are not fully developed and are called blasts or leukemia cells. Lymphomas and leukemias are a part of the broader group of tumors of the hematopoietic and lymphoid tissues.

For the purposes of the present invention, the terms “neoplasm”, “tumor” and “cancer” may be used interchangeably, and they comprise both primary tumors/cancers and secondary tumors/cancers (or “metastases”), as well as mass-forming neoplasms (tumors) and lymphoid neoplasms (such as lymphomas and leukemias), and also MRD.

The term “minimal residual disease” (MRD) refers to the evidence for the presence of small numbers of residual cancer cells that remain in the patient after cancer treatment, e.g. when the patient is in remission (the patient has no symptoms or signs of disease). A very small number of remaining cancer cells usually cannot be detected by routine means because the standard tests used to assess or detect cancer are not sensitive enough to detect MRD. Nowadays, very sensitive molecular biology tests for MRD are available, such as flow cytometry, PCR and next-generation sequencing. These tests can measure minimal levels of cancer cells in tissue samples, sometimes as low as one cancer cell in a million normal cells. In the context of the present invention, the terms “prevention”, “treatment” or “amelioration” of a neoplasm are envisaged to also encompass “prevention, treatment or amelioration of MRD”, whether the MRD was detected or not.

Is it envisaged that the BCMA positive neoplasm of the present invention is a B cell neoplasm or a plasma cell neoplasm. B cells, also known as B lymphocytes, are a type of white blood cell of the lymphocyte subtype. They function in the humoral immunity component of the adaptive immune system by secreting antibodies. Additionally, B cells present antigen (they are also classified as professional antigen-presenting cells) and secrete cytokines. In mammals, B cells mature in the bone marrow, which is at the core of most bones. B cells, unlike the other two classes of lymphocytes—T cells and natural killer (NK) cells—express B cell receptors (BCRs) on their cell membrane. BCRs allow the B cell to bind to a specific antigen, against which it will initiate an antibody response. Plasma cells, also called plasma B cells, plasmocytes, or effector B cells, are white blood cells that secrete large volumes of antibodies. They are usually transported by the blood plasma and the lymphatic system. Plasma cells originate in the bone marrow. B cells differentiate into plasma cells that produce antibody molecules closely modelled after the receptors of the precursor B cell. Once released into the blood and lymph, these antibody molecules bind to the target antigen and initiate its neutralization or destruction.

The level of expression of BCMA on the surface of a cell can be determined e.g. by flow cytometry analysis. The subpopulation of cells (e.g. B cells, plasma cells, MM cells, CD138+ cells) that is selected for analysis of BCMA expression can e.g. be stained with an anti-BCMA antibody, followed by a secondary antibody, and then analyzed in a FACS assay. A BCMA negative cell line (such as K562, A549, TC71, CCRF-CEM) can be used as control. A shift in the FACS assay (with the BCMA negative cell line defining 0% BCMA expression) indicates that the analyzed cells are BCMA positive. Different levels of BCMA expression can exist on the surface cells, such as low, medium or high expression. See also Quinn et al., Blood (2011) 117:890-901 and Sanchez et al, Br J Heamatol 2012 Jul. 18.

The “BCMA positive neoplasm” or the “(BCMA positive) B cell neoplasm or plasma cell neoplasm” can be selected from the group including, but not limited to, multiple myeloma, relapsed and/or refractory multiple myeloma, heavy chain multiple myeloma, light chain multiple myeloma, extramedullary myeloma (extramedullary plasmacytoma, extramedullary multiple myeloma), plasmacytoma, plasma cell leukemia, Waldenstrom's macroglobulinemia (lymphoplasmacytic lymphoma), and smoldering myeloma (smoldering multiple myeloma). The present disclosure hence also relates to a protein for use in the treatment or amelioration of multiple myeloma (MM), plasmacytoma, plasma cell leukemia and Waldenström's macroglobulinemia, as described herein. The MM can be selected from the group consisting of or comprising relapsed and/or refractory multiple myeloma, heavy chain multiple myeloma, light chain multiple myeloma, extramedullary multiple myeloma, and smoldering multiple myeloma.

The “BCMA positive neoplasm” can either be assumed to be BCMA positive, and e.g. be selected from the group of diseases defined herein above (including multiple myeloma and other indications), or the neoplasm is determined to be BCMA positive, e.g. by analyzing the level of BCMA expression on the surface of a selected cell, e.g. as described herein above.

The protein of the invention (and the pharmaceutical composition comprising such protein) is/are useful in the treatment and/or amelioration of the BCMA positive neoplasm as described herein in a subject in need thereof. The term “treatment” refers to both therapeutic treatment and prophylactic or preventative measures. The prophylactic measure usually refers to a situation where the relapse (recidivism/recurrence) of a neoplasm is to be prevented. Treatment includes the administration of the protein (or the pharmaceutical composition comprising such protein) to the patient's body, to an isolated tissue, or to a cell from a patient or a subject in need who has a BCMA positive neoplasm as described herein, a symptom of such neoplasm, or a predisposition toward such neoplasm, with the purpose to cure, heal, alleviate, relieve, alter, remedy, ameliorate, improve, or affect the BCMA positive neoplasm, one or more symptoms of the BCMA positive neoplasm, or the predisposition toward the disease or its recurrence.

The terms “subject in need”, “patient” or those “in need of treatment” include those already with the BCMA positive neoplasm, as well as those in an MRD setting and those in which the neoplasm or its relapse (recidivism/recurrence) is to be prevented. The terms also include human and other mammalian subjects that receive either prophylactic or therapeutic treatment. The “patient” or “subject in need” may hence be a mammalian patient, such as a human patient. The term “prevention” as used herein means the avoidance of the occurrence or of the re-occurrence of a disease as specified herein, by the administration of a protein according to the invention to a subject in need thereof.

The term “amelioration” as used herein refers to any improvement of the disease state (the disease being a BCMA positive neoplasm) of a patient, by the administration of a protein according to the invention to such patient or subject in need thereof. Such an improvement may be seen as a slowing down the progression or stopping the progression of the disease of the patient, and/or as a decrease in severity of disease symptoms, an increase in frequency or duration of disease symptom-free periods or a prevention of impairment or disability due to the disease.

In the case of Multiple Myeloma, the symptoms and signs vary greatly because many organs can be affected by the disease. The common symptoms of multiple myeloma include elevated calcium levels, renal failure, anemia, and bone lesions (together, “CRAB” features). In advanced MM, bone pain, bleeding, and frequent infections may occur. Complications may also include amyloidosis. The International Myeloma Working Group (IMWG) has established criteria for the diagnosis of MM which teach, in addition to the classic CRAB features, three “myeloma defining events” (MDEs):
60% or greater clonal plasma cells on bone marrow examination
Serum involved/uninvolved free light chain ratio of 100 or greater, provided the absolute level of the involved light chain is at least 100 mg/L (a patient's “involved” free light chain—either kappa or lambda—is the one that is above the normal reference range; the “uninvolved” free light chain is the one that is typically in, or below, the normal range)
More than one focal lesion on MRI (magnetic resonance imaging) that is at least 5 mm or greater in size

The presence of at least one of these markers is considered sufficient for a diagnosis of multiple myeloma, regardless of the presence or absence of symptoms or CRAB features. See also Palumbo A. J Clin Oncol. 2014 Feb. 20; 32 (6): 587-600.

Bone pain affects almost 70% of patients and is the most common symptom. It usually involves the spine and ribs. Involvement of the vertebrae may lead to spinal cord compression or kyphosis. Myeloma bone disease is due to the overexpression of receptor activator for nuclear factor KB ligand (RANKL) by bone marrow stroma. RANKL activates osteoclasts, which resorb bone. The resultant bone lesions are lytic (i.e. they cause breakdown) in nature, and are best seen in plain radiographs. The breakdown of bone also leads to the release of calcium into the blood, leading to hypercalcemia and its associated symptoms.

The anemia found in myeloma is usually normocytic and normochromic. It results from the replacement of normal bone marrow by infiltrating tumor cells and inhibition of normal red blood cell production (hematopoiesis) by cytokines.

A bone marrow biopsy can be performed to estimate the percentage of bone marrow occupied by plasma cells. This percentage is used in the diagnostic criteria for MM. Usually, MM patients have ≥10% clonal bone marrow plasma cells. Immunohistochemistry (staining particular cell types using antibodies against surface proteins) can detect plasma cells which express immunoglobulin in the cytoplasm and occasionally on the cell surface; for example, myeloma cells are typically positive for the markers CD56, CD38, CD138. CD319, but other markers may be included as well to define or identify MM.

The so-called “paraprotein” (also called myeloma protein, monoclonal protein or M protein) is an abnormal immunoglobulin fragment that is produced in excess by an abnormal monoclonal proliferation of plasma cells, typically in MM. In theory, MM patients can produce all classes of immunoglobulin, but IgG paraproteins are most common, followed by IgA and IgM, while IgD and IgE myeloma are very rare. In addition, antibody light chains and/or heavy chains may be secreted in isolation: kappa or lambda light chains or any of the five types of heavy chains (alpha, gamma, delta, epsilon or my (μ)-heavy chains). This proliferation of the paraprotein has several deleterious effects on the body, including impaired immune function, abnormally high blood viscosity, and kidney damage. Patients without evidence of paraprotein may have “nonsecretory” myeloma (not producing immunoglobulins); they represent approximately 3% of all MM patients. The presence of serum and/or urinary paraprotein is an indicator for MM, except in patients with true nonsecretory MM. Quantitative measurements of the paraprotein in urine and/or serum of a patient can be used to establish a diagnosis and/or to monitor the disease.

Kidney failure may develop both acutely and chronically. The most common cause of kidney failure in MM is due to proteins secreted by the malignant cells. Myeloma cells produce monoclonal proteins of varying types, most commonly immunoglobulins (antibodies) and free light chains, resulting in abnormally high levels of these proteins (paraproteins) in the blood. Depending on the size of these proteins, they may be excreted through the kidneys, but kidneys can also be damaged by their effects. Furthermore, increased bone resorption leads to hypercalcemia and causes nephrocalcinosis, thereby contributing to the kidney failure.

The most common infections occurring in MM are pneumonias and pyelonephritis. The increased risk of infection is due to immune deficiency. Although the total immunoglobulin level is typically elevated in MM, the majority of the antibodies are ineffective monoclonal antibodies from the clonal plasma cell.

It is envisaged that the administration of the protein in the treatment or amelioration of a BCMA positive neoplasm according to the invention
reduces the level of paraprotein or free light chain in the urine and/or serum by at least about 50% or 55%, at least about 60% or 65%, at least about 70% or 75%, at least about 80% or 85%, or at least about 90% or 95% relative to the paraprotein or free light chain level in the urine and/or serum, respectively, prior to the start of the treatment, i.e. prior to the first administration of the protein of the invention (“prior to”, in this specific context, means within 1, 2, 4, 6, 8, or 12 hours before, within 1, 2, 3, 4, 5 or 6 days before, within 1, 2, 3 or 4 weeks before, or within 1, 2, 3 or 4 months before);
reduces the percentage of plasma cells in the bone marrow by at least about 50% or 55%, at least about 60% or 65%, at least about 70% or 75%, at least about 80% or 85%, or at least about 90% or 95% relative to the percentage of plasma cells in the bone marrow prior to the start of the treatment, i.e. prior to the first administration of the protein of the invention (“prior to”, in this specific context, means within 1, 2, 4, 6, 8, or 12 hours before, within 1, 2, 3, 4, 5 or 6 days before, within 1, 2, 3 or 4 weeks before, or within 1, 2, 3 or 4 months before);
induces a reduction of any of the symptoms described above by at least about 50% or 55%, at least about 60% or 65%, at least about 70% or 75%, at least about 80% or 85%, or at least about 90% or 95% relative to the symptoms prior to the start of the treatment, i.e. to the first administration of the protein of the invention (“prior to”, in this specific context, means within 1, 2, 4, 6, 8, or 12 hours before, within 1, 2, 3, 4, 5 or 6 days before, within 1, 2, 3 or 4 weeks before, or within 1, 2, 3 or 4 months before, depending on the symptom);
inhibits tumor growth or tumor cell proliferation by at least about 50% or 55%, at least about 60% or 65%, at least about 70% or 75%, at least about 80% or 85%, or at least about 90% or 95% relative to untreated patients or relative to untreated cells; and/or
induces lysis of the cells of the BCMA positive neoplasm of at least about 50% or 55%, at least about 60% or 65%, at least about 70% or 75%, at least about 80% or 85%, or at least about 90% or 95% relative to untreated patients or relative to untreated cells.

The ability of a protein of the invention to inhibit tumor growth/tumor cell proliferation or to induce cell lysis may be evaluated in an animal model predictive of efficacy in human tumors, or in an in vitro or ex vivo study (such as depletion of BCMA positive cells by autologous T cells from a multiple myeloma patient's BM aspirate induced by the protein). Efficacy assessments of the protein may furthermore be performed as follows: Tumor assessment can be done by analysis of percent myeloma involvement, by FISH (fluorescent in situ hybridization) as well as by karyotyping in the bone marrow (BM). Data for BM karyotyping and FISH may be obtained from a BM sample. Serum protein electrophoresis (SPEP) and urine protein electrophoresis (UPEP) allow for measurement of serum/urine M protein. Immunofixation is another means to detect serum and/or urine M protein. It is also envisaged that serum free light chain assay and ratio analysis can be performed. In case of free light chain (FLC) multiple myeloma, FLC can be analyzed in serum and urine (sFLC and uFLC). Levels of involved/uninvolved FLC, ratio of monoclonal lambda-FLC/kappa-FLC, and ratio of monoclonal kappa-FLC/lambda-FLC can be determined. Furthermore, quantitative and qualitative immunoglobulin (Ig) can also be analyzed, and beta-2 microglobulin in serum can be assessed. It is also envisaged that skeletal survey and plasmacytoma assessments can be performed. Screening imaging to evaluate for extramedullary relapse using whole-body MRI or PET/CT can be performed. Imaging appropriate for assessment of bone lesions includes, but is not limited to, CT scan, MRI, PET, PET-CT, or other standard-of-care method. It is also envisaged that minimal residual disease is measured by a next generation sequencing (NGS) based assay. For this purpose, bone marrow aspirates can be collected from subjects suspected to be complete responders. Plasma samples can additionally be collected from subjects at the same time points as BM MRD samples are collected, to assess the feasibility of MRD detection on ctDNA (circulating tumor DNA). MRD response may be defined as <1 tumor cell/10⁴ normal cells in the bone marrow per FACS using antibodies to cytIgλ, cytIgκ, CD19, CD56 or CD138, CD38, and CD45, as needed. In one embodiment of the invention, these markers are a sufficient condition to define a tumor cell in the context of the present invention.

It is envisaged that a patient's or subject's response to the administration of the protein according to the invention is measured in one of the following ways:
quantitative measurement of the paraprotein (M protein) or free light chain in the urine and/or serum;
determination of the percentage of plasma cells in the bone marrow; and/or
imaging (CT, MRI) of extramedullary manifestations.

The invention provides a protein comprising a first domain which binds to BCMA, a second domain which binds to CD3 and a third domain which extends the half-life of the protein, for use in the treatment or amelioration of a BCMA positive neoplasm, wherein the protein is administered in a first cycle comprising:
administering a first dose of the protein on day 1, and
administering a second dose of the protein on a day after day 1 and before day 8, wherein the second dose exceeds the first dose and is at least about 7 mg/day.

It is furthermore envisaged that the first cycle comprises:
administering a target dose of the protein after the administration of the second dose, wherein the target dose is identical to or exceeds the second dose and is from about 9 mg/day to about 24 mg/day, preferably from 12.5 mg/day to about 24 mg/day.

A “treatment cycle” or “cycle” refers to a period of administration of the protein at specific dosages and dosing intervals. The protein of the invention is administered in at least one cycle, but more cycles of administration such as 2, 3, 4, 5, 6, 7, 8, 9, 10, 11, 12, 13, 14, 15, 16, 17, 18, 19, 20, 21, 22, 23, 24, 25 or more are also envisaged. The term “at least one cycle” means “one or more cycles”. According to the invention, it is envisaged that “one cycle” (i.e. the first cycle, the second cycle and/or any subsequent cycle) corresponds to a period of about 25 to about 30 or 31 days, of about 26 or 27 to about 29 days, preferably of about 28 days or four weeks. A cycle begins with the first administration of the protein of the invention on “day 1” of such cycle. One cycle may immediately be followed by a subsequent cycle, but an administration-free interval between two cycles is also envisaged. The different administration cycles are not envisaged to be fully identical. In particular, it is envisaged that the first cycle differs from subsequent cycles, in particular in terms of the dosing regimen of the protein. The first cycle is designed to comprise a step dosing of the protein until the protein's target dose is reached. It is envisaged that the target dose reached in the first cycle, usually within the first 10 days, will be used (administered) during the completion of the first cycle and during the subsequent cycle(s). The first cycle may hence be regarded as an “initiation cycle”, and subsequent cycles as “maintenance cycles”.

An initiation cycle is preferably administered to a patient as the first cycle when the patient begins a course of treatment with the protein. An initiation cycle may also be administered to a patient when the patient re-starts a course of treatment with the protein, for example, following a treatment-free period, dosing interruption (e.g. when a patient didn't complete a previous treatment cycle), or a relapse or progression of a neoplasm in the patient. Although administration of one initiation cycle will typically be sufficient, in some embodiments of the methods of the invention, administration of two or more initiation cycles is contemplated. In one particular embodiment, only one initiation cycle is administered to the patient.

Multiple maintenance cycles (e.g. 2, 3, 4, 5, 6, 7, 8, 9, 10, 11, 12 or more cycles) can be administered to the patient depending on the desired duration of treatment for that patient. For instance, the patient may receive maintenance cycles of the protein until the patient achieves a desired level of response, such as a complete response, very good partial response or partial response. In some embodiments, two or more maintenance cycles are administered to the patient. In other embodiments, four or more maintenance cycles are administered to the patient. In still other embodiments, six to twelve maintenance cycles are administered to the patient. In certain embodiments, the maintenance cycles are administered consecutively with no treatment-free periods between the maintenance cycles. If a treatment interruption is necessary, ideally the duration of the treatment-free period will be no greater than twice the dosing interval employed in the maintenance cycle. By way of example, if the dosing interval employed in the maintenance cycle is once weekly, the treatment-free period between maintenance cycles will preferably be about two weeks or less.

A “step” in the dosing of the protein or a “dose step” means that there is an increase in the dose to be administered from one administration (on one day within a cycle, usually the first cycle) to a subsequent administration (on a later day within such cycle). One dose step within a cycle means that one dose to be administered on one day is followed by a higher dose to be administered on a subsequent day. Accordingly, two dose steps within a cycle mean that one dose (e.g. the first dose to be administered on day 1) is followed by a second dose which is higher than the first dose and is administered on a subsequent day, and the second dose is followed by a third dose (e.g. the target dose) which is higher than the second dose and is administered on a subsequent day. In the case of one or two steps, (at least) the last dose to be administered during the first cycle is the so-called “target dose”. The first dose to be administered on day 1 may be regarded as a “priming dose” or a “run-in dose”, and the “target dose” may be regarded as the “therapeutic dose”. The target dose is hence the highest dose to be administered in a cycle. In other words, the target dose exceeds the second dose and as a consequence also exceeds the first dose. The target dose also exceeds the (optional) third dose.

During the first cycle, a first dose of the protein is administered on day 1. It is envisaged that the first dose is at least about 800 μg/day. The first dose of the protein may be from about 800 μg/day to about 1200 μg/day, from about 800 μg/day to about 1100 μg/day, from about 800 μg/day to about 1000 μg/day or from about 800 μg/day to about 900 μg/day. The first dose may be 800 μg/day.

It is envisaged that during the first cycle of administration, a second dose of the protein is administered on a day after day 1 (i.e. after the administration of the first dose) and before day 8 or before the administration of the target dose, wherein the second dose exceeds the first dose. According to one aspect of the invention, during the first cycle of administration, a second dose of the protein is administered on a day after day 1 (i.e. after the administration of the first dose) and before day 8 (i.e. before the administration of the target dose), wherein the second dose exceeds the first dose and is at least about 7 mg/day. The term “a dose of at least (about) x mg/day” means a “minimum dose of (about) x mg/day”. A dose to be administered according to this teaching may not substantially go below this limit. The second dose may be administered on day 2, day 3, day 4, day 5, day 6, or day 7. In particular, the second dose is administered on day 3 or day 4, preferably on day 3. The second dose may be from about 4 mg/day to about 12.5 mg/day, from about 4.5 mg/day to about 12 mg/day, from about 4 mg/day to about 10 mg/day, from about 4.5 mg/day to about 9 mg/day, from about 4 mg/day to about 8 mg/day, from about 4 mg/day to about 7 mg/day, from about 4 mg/day to about 6.5 mg/day, from about 4.5 mg/day to about 6 mg/day. In one embodiment of the invention, the second dose is from about 4 mg/day to about 10 mg/day, preferably from about 4 mg/day to about 7 mg/day, and is administered on day 3. The second dose may also be from about 7 mg/day to about 18 mg/day, from about 7.5 mg/day to about 15 mg/day, from about 8 mg/day to about 12 mg/day, from about 8.5 mg/day to about 10 mg/day or from about 9 mg/day to about 9.5 mg/day. In one embodiment of the invention, the second dose is from about 8 mg/day to about 10 mg/day, preferably about 9 mg/day, and is administered on day 3.

According to one aspect of the invention, during the first cycle of administration, a third dose of the protein is administered on a day after the day of administration of the second dose and before the day of administration of the target dose, wherein the third dose exceeds the second dose. It is envisaged that the third dose is from about 7 mg/day to about 12 mg/day, from about 7 mg/day to about 11 mg/day, from about 8 mg/day to about 10 mg/day or about 9 mg/day. The third dose may be administered on day 5, day 6 or day 7. For example, the third dose may be from about 8 mg/day to about 10 mg/day (preferably about 9 mg/day) and is administered on day 5.

The target dose of the protein to be administered is envisaged to be from about 9 mg/day to about 24 mg/day or from 12.5 mg/day to about 24 mg/day. The target dose may also be from about 14 mg/day to about 22 mg/day, from about 15 mg/day to about 21 mg/day, from about 16 mg/day to about 20 mg/day, from about 17 mg/day to about 19 mg/day, or about 18 mg/day. In the course of the first cycle, the target dose is administered for the first time on a day from day 6 to day 10, preferably on a day from day 7 to day 9, more preferably on day 8. For example, the target dose is from about 16 mg/day to about 20 mg/day and is administered on day 8. Once the target dose is reached (=administered for the first time) on a day from day 6 to day 10 of the first cycle, it is furthermore envisaged that the target dose is administered on day 15 (+/− one or two days) and day 22 (+/− one or two days). In other words, is preferably administered once every 7 days.

In one embodiment of the invention, the first cycle comprises:
administering a first dose of the protein of about 800 μg/day to about 1000 μg/day, preferably about 800 μg/day,
administering a second dose of the protein of about 8 mg/day to about 10 mg/day, preferably about 9 mg/day, and
administering a target dose of the protein of about 12 mg/day to about 20 mg/day, preferably about 16 mg/day to about 20 mg/day, such as 18 mg/day.

According to this embodiment, it is also envisaged that the first dose is administered on day 1, the second dose is administered on day 3 or 4, preferably on day 3, and the target dose is administered on day 8 (+/− one day), day 15 (+/− one day) and day 22 (+/− one day), preferably on day 8, day 15 and day 22.

In another embodiment of the invention, the first cycle comprises:
administering a first dose of the protein of about 800 μg/day to about 1000 μg/day, preferably about 800 μg/day,
administering a second dose of the protein of about 4 mg/day to about 10 mg/day, preferably about 4 mg/day to about 7 mg/day, or 4.5 mg/day to 6 mg/day,
optionally administering a third dose of the protein of about 8 mg/day to about 10 mg/day, preferably about 9 mg/day, wherein the third dose exceeds the second dose, and
administering the target dose of the protein of about 14 mg/day to about 22 mg/day, preferably about 16 mg/day to about 20 mg/day, such as 18 mg/day.

According to this embodiment, it is also envisaged that the first dose is administered on day 1, the second dose is administered on day 3 or 4, preferably on day 3, the optional third dose is administered on day 5 or day 6, preferably on day 5, and the target dose is administered on day 8 (+/− one day), day 15 (+/− one day) and day 22 (+/− one day), preferably on day 8, day 15 and day 22.

According to one aspect of the invention, the protein in administered in the first cycle as follows: The first dose of 800 μg/day is administered on day 1, the second dose of 6 mg/day is administered on day 3, and the target dose of 18 mg/day is administered on day 8, day 15 and day 22. According to another aspect of the invention, the protein in administered in the first cycle as follows: The first dose of 800 μg/day is administered on day 1, the second dose of 4.5 mg/day is administered on day 3, the third dose of 9 mg/day is administered on day 5, and the target dose of 18 mg/day is administered on day 8, day 15 and day 22.

According to the invention, one cycle (optionally having a length of about 28 days, see above) comprises at least three individual administrations of the protein, preferably it comprises or consists of three to six or four to six individual administrations, such as four or five or six individual administrations. This means that the subject in need receives the protein of the invention at least three times during a cycle, preferably four or five or six times. For example, if a cycle consists of four weeks, the protein may be administered to the subject in need once per week. This applies in particular for the second and/or any subsequent cycle. An “individual administration” hence means the administration of a specified dose as defined above on one day. Within one cycle, such administration or individual administration is followed by a period (of usually at least one day and usually up to six days) in which the protein is not administered. It is also envisaged that the first cycle comprises or consists of three to six individual administrations, preferably four or five or six individual administrations of the protein. It is envisaged that the last two or three administrations within the first cycle are administrations of the protein at the target dose.

It is envisaged that the protein is administered during the second cycle and optionally during any subsequent cycle at constant doses which correspond to the target dose. No dose steps are envisaged for the second cycle and optionally for any subsequent cycle. The target dose is envisaged to be from about 9 mg/day to about 24 mg/day or from 12.5 mg/day to about 24 mg/day. The target dose may also be from about 14 mg/day to about 22 mg/day, from about 15 mg/day to about 21 mg/day, from about 16 mg/day to about 20 mg/day, from about 17 mg/day to about 19 mg/day, or about 18 mg/day. The second cycle and optionally the further subsequent cycles may comprise administering the protein at the target dose on day 1, day 8 (+/− one or two days), day 15 (+/− one or two days) and day 22 (+/− one or two days). In particular, the second cycle and optionally any further subsequent cycles comprise administering the protein at the target dose once every 7 days, i.e., in weekly intervals. The term “dx (+/− one or two days)” means any day selected from day x minus two days, day x minus one day, day x, day x plus one day and day x plus two days. For example, if day x is d8, then “d8 (+/− one or two days)” means any day from d6 to d10, i.e. d6, d7, d8, d9 or d10. Likewise, “d8 (+/− one day)” means any day from d7 to d9, i.e. d7, d8 or d9.

It is envisaged that the protein to be administered according to the invention has a molecular weight of about 75 kDa to about 200 kDa, about 80 kDa to about 175 kDa, about 85 kDa to about 150 kDa, about 90 kDa to about 130 kDa, about 95 kDa to about 120 kDa, and preferably about 100 kDa to about 115 kDa or about 105 kDa to about 110 kDa.

The protein of the present invention comprises a domain (the “third domain”) which extends or enhances the half-life or the elimination half-life of the construct. The term “extends” or “enhances” may be defined with respect to or in comparison to the same protein not comprising such a domain (an “HLE domain”). It is envisaged that the half-life or the elimination half-life of the protein of the invention is at least about five times longer than the half-life or the elimination half-life of the protein not comprising the third domain, or at least about ten times longer, or at least about 15 times longer or at least about 20 times longer.

The third domain may be defined as a domain which provides the protein to be administered according to the invention with a “half-life” or “terminal half-life” or “elimination half-life” (T_{1/2}) of about 3 to about 14 days, about 4 to about 12 days, about 3 or 4 to about 10 days, about 3 or 4 to about 8 days, about 3 days to about 5 days, or about 5 days to about 6 or about 7 days. “Half-life” is the time required for a quantity to reduce to half its initial value. The medical sciences refer to the half-life of substances or drugs (here: the protein) in the human body, e.g. in the serum or in the plasma. Therefore, the “half-life” is sometimes also referred to as “serum half-life” or “plasma half-life”. It can be determined by measuring the concentration of the administered protein in the serum/plasma of a subject. Typically, the elimination or removal of an administered substance/drug refers to the body's cleansing through biological processes such as metabolism, excretion, also involving the function of kidneys and liver. “Elimination half-life” may be defined as the time required for the concentration of the drug (here: the protein) to reach half of its original value.

The protein of the invention will generally be designed for specific routes and methods of administration. A route of administration in pharmacology is the path by which a substance is taken into the body. Routes of administration are generally classified by the location at which the substance is applied, these can be topical (local), enteral (system-wide effect of the substance, but delivered through the gastrointestinal tract), or parenteral (systemic action of the substance, but delivered by routes other than the gastrointestinal tract). In general, parenteral administration includes, but is not limited to, intravenous (IV), intracerebral, intraarterial, intraperitoneal, intraosseous, and intravesical delivery. The reason for the choice of routes of drug administration are governed by various factors such as:
Physical and chemical properties of the drug. The physical properties are solid, liquid and gas. The chemical properties are solubility, stability, pH, irritancy etc.
Site of desired action: The action may be localized and approachable or generalized and not approachable.
Rate of extent of absorption of the drug from different routes.
Condition of the patient.

The protein of this invention (and a pharmaceutical composition comprising this protein) is particularly useful for parenteral administration. Parenteral administration generally acts more rapidly than topical or enteral administration, and often comes along with a very high bioavailability of up to 100% (in particular, in the case of IV administration). The administration according to the present invention is preferably intravenous. Parenteral or intravenous administration can be performed by injection (e.g. using a needle and a syringe) or by infusion (e.g. via a catheter and a pump system). It is hence envisaged that the administration according to the present invention is via intravenous injection or via intravenous infusion. Usually, an IV infusion is administered via a line, a port or a catheter (small, flexible tube), such as a central venous access or a central venous catheter (CVC) which is a catheter placed into a large vein, or a peripheral venous catheter (PVC), which is a catheter placed into a peripheral vein. In general, catheters or lines can be placed in veins in the neck (internal jugular vein), chest (subclavian vein or axillary vein), groin (femoral vein), or through veins in the arms (also known as a PICC line, or peripherally inserted central catheters). Central IV lines have their catheters that are advanced through a vein and empty into a large central vein, usually the superior vena cava, inferior vena cava or even the right atrium of the heart. A peripheral intravenous (PIV) line is used on peripheral veins (the veins in the arms, hands, legs and feet). A port is a central venous line that does not have an external connector; instead, it has a small reservoir that is covered with silicone rubber and is implanted under the skin. Medication is administered intermittently by placing a small needle through the skin, piercing the silicone, into the reservoir. When the needle is withdrawn, the reservoir cover reseals itself. The cover can accept hundreds of needle sticks during its lifetime.

The present invention also provides for a bolus administration of the protein of the invention. A bolus is the administration of a discrete amount of a medication, drug, or other compound within a specific negligible time, generally within 1-30 minutes. In most cases, the bolus administration is given intravenously. A bolus is usually administered via injection (e.g. an intravenous bolus injection), but a bolus infusion (e.g. an intravenous bolus infusion) is also possible. A short-term infusion is an infusion (usually an IV infusion), of a relatively small volume (such as 50 mL to 500 mL, or 100 mL to 250 mL), which is administered over a period of, at most, three hours, usually of 30 to 60 minutes or about 60 minutes. A short-term (or short-term IV) infusion of the protein is envisaged by the present invention.

Intravenous “intermittent infusion” is an infusion of a volume of medication over a set period of time, such as 20-120 minutes or 30-60 minutes, at prescribed intervals, such as every 4, 6, 8, 10, 12, 14, 16, 18, 20, 22 or 24 hours. The purpose is to administer small amounts of medication at regular intervals. An intermittent medication—like any other form of infusion—may be administered by gravity or via an electronic infusion device (EID), also known as an infusion pump.

In the case of an infusion, an infusion pump may be used to infuse the medication (protein) into a patient's circulatory system. The pump is generally used intravenously, although arterial and epidural infusions with pumps are also possible. The solution for infusion may be prepared in bags for IV infusion and delivered through infusion lines. Infusion pumps can administer fluids in ways that would be unreliable if performed manually. For example, they can administer as little as 0.1 mL per hour injections, injections every minute, injections with repeated boluses, up to a maximum number per hour, or fluids whose volumes vary by the time of day. It is also possible that infusions are administered using only the pressure supplied by gravity. Different types of infusions according to the present invention include, but are not limited to, bolus infusion, short-term infusion, and intermittent infusion. The protein of the present invention may hence be administered e.g. as a bolus administration (bolus injection or bolus infusion), as an injection, or as a short-term infusion, for example over a period of about 30 to about 90 minutes or about 60 minutes.

Pharmaceutical compositions may be administered using a medical device. Examples of medical devices for administering pharmaceutical compositions are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163.

In the context of the present invention, it is envisaged that a premedication is administered prior to each administration (or “individual administration”) of the protein, in particular during the first cycle. It is envisaged that “prior to”, in this specific context, means within 24 hours, 18 hours, twelve hours, six hours, five hours, four hours, or three hours, and preferably within 120, 90, 60 or 30 minutes before the administration of the protein. The premedication may e.g. be administered 30-120 or 30-60 minutes prior to the administration of the protein. It is also envisaged that a comedication is administered concurrent with or after the start of administration of the protein in the first cycle, and optionally also concurrent with or after the start of administration of the protein in one or more of the following cycles, as needed by the patient. It is envisaged that “after”, in this specific context, means within 24 hours, 18 hours, twelve hours, six hours, five hours, four hours, or three hours, and preferably within 120, 90, 60, 30, 20, 15 or 10 minutes after the start of administration of the protein. The comedication may e.g. be administered 10-120, 10-60, 10-30 or 15-20 minutes after start of administration of the protein. The purpose of the premedication or comedication may be e.g. to prevent or reduce severity of infusion-related reactions. Premedication is preferred over comedication.

The premedication or comedication may include any one or a combination of the following:
Paracetamol (acetaminophen, APAP) or an equivalent; to be preferably administered orally (p.o.) or intravenously; and to be preferably administered at a dose of 100-4000 mg, preferably 200-3000 mg or 300-2500 mg or 400-2000 mg or 500-1500 mg, preferably 600-1400 mg, 700-1300 mg, 800-1200 mg, 900-1100 mg or about 1000 mg p.o. paracetamol (or an equivalent dose for an equivalent medication and/or another route of administration). The skilled person knows how to identify paracetamol equivalents. They include, but are not limited to, ibuprofen (to be administered e.g. at a dose of 100-3200 mg, preferably 200-3000 mg or 300-2500 mg or 400-2000 mg, preferably 500-1500 mg, 600-1200 mg, 700-1000 mg, 750-900 mg or about 800 mg) and metamizole (to be administered e.g. at a dose of 100-4000 mg, preferably 200-3000 mg or 300-2500 mg or 400-2000 mg, or about 500-1000 mg).

One or more analgesics selected from meperidine, dipyrone, hydromorphone, fentanyl, and tramadol
Antihistamine, to be preferably administered orally or intravenously, and to be preferably administered at a dose equivalent to diphenhydramine 50 mg i.v. The skilled person knows how to identify antihistamines. They include, but are not limited to, antihistamines of oral, parenteral or rectal route such as: azatadine (maximum dose e.g. 4 mg/day), brompheniramine (maximum dose e.g. 30 mg/day), cetirizine (maximum dose e.g. 15 mg/day), chlorpheniramine (maximum dose e.g. 30 mg/day), clemastine (maximum dose e.g. 10 mg/day), cyproheptadine (maximum dose e.g. 15 mg/day), desloratadine (maximum dose e.g. 7 mg/day), dexchlorpheniramine (maximum dose e.g. 15 mg/day), diphenhydramine (maximum dose e.g. 350 mg/day), doxylamine (maximum dose e.g. 180 mg/day), fexofenadine (maximum dose e.g. 200 mg/day), loratadine (maximum dose e.g. 15 mg/day), phenindamine (maximum dose e.g. 180 mg/day)
Glucocorticoid, to be preferably administered orally or intravenously, and to be preferably administered at a dose equivalent to 2-20 mg or 4-16 mg or 6-12 mg or 8 mg dexamethasone i.v. (the equivalence referring to the glucocorticoid potency)

All four substances or substance groups listed above may be administered as premedication or comedication, or a combination of only two or a combination of three substances, or only one of the four substances. It is envisaged that the glucocorticoid (GC) dose administered before the start of the second cycle may be identical to the GC dose administered before start of the first cycle, or may be reduced to about 50% of the dose administered before start of the first cycle, or may be omitted for the second (and/or potentially any subsequent) cycle. A reduction of the GC dosage may apply e.g. if the protein according to the invention is well tolerated without significant signs of infusion-related reactions during the first cycle. It is furthermore envisaged that the dose may further be reduced before start of the third and any subsequent cycle. Alternatively, while GC is administered as premedication (and potentially comedication) before the start of the first cycle, no GC premedication or comedication is administered in the second, third, fourth and/or fifth cycle. In general, the dose of the premedication or comedication that is to be used in accordance with the embodiments of the present invention will depend on the circumstances of the individual patient.

Glucocorticoids are a class of corticosteroids, which are a class of steroid hormones. Glucocorticoids are corticosteroids that bind to the glucocorticoid receptor. A less common synonym is glucocorticosteroid. Cortisol (known as hydrocortisone when used as a medication) is the most important human glucocorticoid. A variety of synthetic glucocorticoids, some far more potent than cortisol, have been created for therapeutic use. They differ in both pharmacokinetics (e.g. absorption factor, half-life, volume of distribution, clearance) and pharmacodynamics (e.g. glucocorticoid potency or mineralocorticoid potency). Cortisol is the standard of comparison for glucocorticoid potency. Corticosteroid conversion calculators or equivalency tables are available in the internet. One example for commonly prescribed replacement steroid equivalents may be prednisone (5 mg)=cortisone (25 mg)=dexamethasone (0.75 mg)=hydrocortisone (20 mg)=methylprednisolone (4 mg). These doses indicate the equivalent pharmacologic dose of systemic glucocorticoids. Another corticosteroid comparison chart indicating the half-lives of the different substances can be found e.g. in: https://www.ncbi.nlm.nih.gov/books/NBK279156/table/adrenal_glucocorticoid-therapy-and-adrenal-suppression.T./.

Examples of GCs to be used as premedication or comedication in the present embodiment include, but are not limited to, cortisone, hydrocortisone, prednisone, prednisolone, methylprednisolone, dexamethasone, betamethasone, beclomethasone, budesonide, triamcinolone, cloprednol, deflazacort, fluocortolone, cortivazol, paramethasone, fluticasone, fluticasone propionate, triamcinolone acetonide, as well as combinations and/or pharmaceutically acceptable derivatives thereof. In the context of the present invention, the different GCs may be used alone or in combination. Dexamethasone, prednisone and prednisolone are preferred embodiments of GCs.

It is envisaged that all substances which already are or will be classified as a “glucocorticoid” may be employed in the context of the present invention as well. Such future glucocorticoids include compounds which specifically bind to and activate the glucocorticoid receptor. The term “specifically binds to the GC receptor” means in accordance with the present invention that the GC (or a compound which is assumed to act like a GC) associates with (e.g., interacts with) the GC receptor (also known as NR3C1) to a statistically significant degree as compared to association with proteins/receptors generally (i.e., non-specific binding). When the GC receptor binds to glucocorticoids, its primary mechanism of action is the regulation of gene transcription. In the absence of GC, the glucocorticoid receptor (GR) resides in the cytosol complexed with a variety of proteins including heat shock protein 90 (hsp90), heat shock protein 70 (hsp70) and the protein FKBP52 (FK506-binding protein 52). Binding of the GC to the glucocorticoid receptor results in release of the heat shock proteins. It is thus envisaged that a future GC, or a pharmaceutically acceptable derivative or salt of a GC, is able to bind to the GC receptor and to release the above mentioned heat shock proteins. The activated GR complex then up-regulates the expression of anti-inflammatory proteins in the nucleus or represses the expression of pro-inflammatory proteins in the cytosol by preventing the translocation of other transcription factors from the cytosol into the nucleus.

The protein of the present invention comprises a first domain which binds to BCMA, a second domain which binds to CD3 and a third domain which extends/enhances the half-life of the protein. As explained in more detail below, the protein may be in the format of a single chain polypeptide. It is also envisaged that the protein consists of more than one polypeptide chain. In this case, the first domain (which binds to BCMA) may be composed of two polypeptide chains of parts thereof, and likewise the second domain (which binds to CD3) may be composed of two polypeptide chains of parts thereof. The third domain (which extends the half-life) may also be composed of two polypeptide chains of parts thereof.

Peptides are short chains of amino acid monomers linked by covalent peptide (amide) bonds. Hence, peptides fall under the broad chemical classes of biological oligomers and polymers. Amino acids that are part of a peptide or polypeptide chain are termed “residues” and can be consecutively numbered. All peptides except cyclic peptides have an N-terminal residue at one end and a C-terminal residue at the other end of the peptide. An oligopeptide consists of only a few amino acids (usually between two and twenty). A polypeptide is a longer, continuous, and unbranched peptide chain. Peptides are distinguished from proteins on the basis of size, and as an arbitrary benchmark can be understood to contain approximately 50 or fewer amino acids. A protein consists of one or more polypeptides or polypeptide chains, usually arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute. Although the terms polypeptide and protein are sometimes used interchangeably, a polypeptide is technically a polymer of amino acids, whereas the term protein is used for a polypeptide or polypeptides that have folded properly and are functional.

A protein may consist of one or more polypeptides. Polypeptides may hence form multimers such as dimers, trimers and higher oligomers, which consist of more than one polypeptide/polypeptide chain. Polypeptides forming such dimers, trimers etc. may be identical or non-identical. The corresponding structures of higher order of such multimers are, consequently, termed homo- or heterodimers, homo- or heterotrimers etc. An example for a hereteromultimer is an antibody or immunoglobulin molecule, which, in its naturally occurring form, consists of two identical “light chains” and two identical “heavy chains”. The terms “peptide”, “polypeptide” and “protein” also refer to naturally modified peptides/polypeptides/proteins wherein the modification is accomplished e.g. by post-translational modifications like glycosylation, acetylation, phosphorylation and the like. A “peptide”, “polypeptide” or “protein” when referred to herein may also be chemically modified such as pegylated. Such modifications are well known in the art.

As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (u), delta (A), gamma (γ), alpha (a), and epsilon (¿), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein. From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp. Immunol. 29:185-203; 2005) and AHo (Honegger and Pluckthun, J. Mol. Biol. 309 (3): 657-670; 2001).

Within the definition of “antibody” according to the invention are full-length antibodies, also including camelid antibodies and other immunoglobulins generated by biotechnological or protein engineering methods or processes. These full-length antibodies may be for example monoclonal, recombinant, chimeric, deimmunized, humanized and human antibodies, as well as antibodies from other species such as mouse, hamster, rabbit, rat, goat, or non-human primates.

The term “binding domain” or “domain which binds to . . . ” characterizes in connection with the present invention a domain of the protein which immunospecifically binds to/interacts with/recognizes a given target or antigen (here: BCMA in the case of the first domain, and CD3 in the case of the second domain). The structure and function of the first domain (binding to BCMA), and preferably also the structure and/or function of the second domain (binding to CD3), is/are based on the structure and/or function of an antibody, e.g. of a full-length immunoglobulin molecule. The first and/or second (binding) domain of the protein hence comprises a paratope, also called an “antigen-binding site”, which is defined as the part of an antibody which recognizes and binds to an antigen and more specifically to the epitope within the antigen. A paratope is usually a small region within an antibody's Fab region. The Fab is composed of two variable domains (VH in the heavy chain and VL in the light chain) and two constant domains (CH1 and CL). In the pairing of light and heavy chains, the two variable domains dimerize to form the Fv fragment which contains the antigen binding site. Within each variable domain lie six hypervariable loops, three in the light chain (L1, L2, and L3) and three in the heavy chain (H1, H2, and H3), supported by a framework of β-sheets. The six hypervariable loops within the variable domains of antibodies are commonly termed complementarity determining regions (CDRs). The light and heavy variable domains fold in a manner that brings the hypervariable loops together to create the antigen-binding site or paratope. It is a common practice to identify a paratope through the identification of CDRs.

The “binding domain” or “domain which binds to . . . ” may hence comprise the minimum structural requirements of an antibody which allow for immunospecific target binding. This minimum structural requirement of the first domain may e.g. be defined by the presence of at least three light chain CDRs (i.e. CDR1, CDR2 and CDR3 of the VL region) and/or of three heavy chain CDRs (i.e. CDR1, CDR2 and CDR3 of the VH region), preferably of all six CDRs. It is envisaged that the second domain also comprises this minimum structural requirement of an antibody which allow for the immunospecific target binding. More preferably, the second domain also comprises at least three light chain CDRs (i.e. CDR1, CDR2 and CDR3 of the VL region) and/or three heavy chain CDRs (i.e. CDR1, CDR2 and CDR3 of the VH region), preferably all six CDRs. A “domain which binds to” (or a “binding domain”) may typically comprise an antibody light chain variable region (VL) and an antibody heavy chain variable region (VH); however, it does not have to comprise both, but may comprise only one of VH or VL. Fd fragments, for example, often retain some antigen-binding function of the intact antigen-binding domain. It is also envisaged that the first and/or second domain are composed of two heavy chain variable regions which are arranged in sequence and provide for bivalent binding to the respective target.

Examples for the format of a “domain which binds to” (or a “binding domain”) include, but are not limited to, fragments of full-length antibodies (such as VH, VHH, VL), (s)dAb, Fv, light chain (VL-CL), Fd (VH-CH1), heavy chain, Fab, Fab′, F(ab′)₂ or “r IgG” (“half antibody” consisting of a heavy chain and a light chain), antibody variants or derivatives such as scFv, di-scFv or bi(s)-scFv, scFv-Fc, scFv-zipper, scFab, Fab₂, Fab₃, diabodies, single chain diabodies, tandem diabodies (Tandab's), tandem di-scFv, tandem tri-scFv. “minibodies” (selected from formats such as (VH-VL-CH3)₂, (scFv-CH3)₂, ((scFv)₂-CH3+CH3)), ((scFv)₂-CH3) or (scFv-CH3-scFv)₂, multibodies such as triabodies or tetrabodies, and single domain antibodies such as nanobodies or single variable domain antibodies comprising merely one variable region, which might be VHH, VH or VL. Further examples for the format of a “domain which binds to” (or a “binding domain”) include (1) an antibody fragment or variant comprising VL, VH, CL and CH1 (such as Fab); (2) an antibody fragment or variant comprising two linked Fab fragments (such as a F(ab′)₂); (3) an antibody fragment or variant comprising VH and CH₁ (such as Fd); (4) an antibody fragment or variant comprising VL and CL (such as the light chain); (5) an antibody fragment or variant comprising VL and VH (such as Fv); (5) a dAb fragment (Ward et al., (1989) Nature 341:544-546), which has a VH domain; (6) an antibody variant comprising at least three isolated CDRs of the heavy and/or the light chain; and (7) a single chain Fv (scFv). Further examples for embodiments of binding domains are e.g. described in WO 00/006605, WO 2005/040220, WO 2008/119567, WO 2010/037838. WO 2013/026837, WO 2013/026833, US 2014/0308285, US 2014/0302037, W O2014/144722, WO 2014/151910, and WO 2015/048272.

The protein to be administered according to the present invention comprises a first domain which binds to BCMA and a second domain which binds to CD3 and is hence “at least bivalent” and “at least bispecific”. In certain embodiments, the protein used in the methods of the invention is multivalent. The valency of the binding protein denotes the number of individual antigen-binding domains within the protein. For example, the terms “monovalent.” “bivalent,” and “tetravalent” with reference to the protein in the context of the invention refer to proteins with one, two, and four antigen-binding domains, respectively. Thus, a multivalent protein comprises two or more antigen-binding domains. A protein according to the invention can have more antigen-binding domains (e.g. a higher valency) than specificities. For instance, in a case where it has two binding domains for the first target (BCMA) and one binding domain for the second target (CD3)—or vice versa—the construct would be (at least) trivalent and bispecific. The protein may hence be bivalent, trivalent, tetravalent or multivalent/polyvalent.

The terms “(specifically or immunospecifically) binds to”. “(specifically or immunospecifically) recognizes”, or “(specifically or immunospecifically) reacts with” mean in accordance with this invention that a binding domain or a protein comprising such binding domain interacts or (immuno-) specifically interacts with a given epitope on the target molecule (antigen), here: BCMA and CD3, respectively. This interaction or association occurs more frequently, more rapidly, with greater duration, with greater affinity, or with some combination of the aforementioned, to an epitope on the specific target than to alternative substances (non-target molecules). Because of the sequence similarity between homologous proteins in different species, a binding domain or a protein comprising such binding domain which immunospecifically binds to its target (such as a human target) may, however, cross-react with homologous target molecules from different species (such as, from non-human primates, e.g. macaque). The term “specific/immunospecific binding” can hence include the binding of a binding domain or a protein comprising such binding domain to epitopes or structurally related epitopes in more than one species.

In the context of the present invention, the term “epitope” refers to the region or molecular structure within the antigen that makes specific contacts with the paratope and is hence recognized/immunospecifically recognized by the binding domain comprising such paratope. An “epitope” is antigenic, and thus the term epitope is sometimes also referred to as “antigenic structure” or “antigenic determinant”. Specific binding is believed to be accomplished by specific motifs in the amino acid sequence of the binding domain and the antigen. Thus, binding is achieved as a result of their primary, secondary and/or tertiary structure as well as the result of potential secondary modifications of said structures. Many tools exist for the identification of antibody epitopes (such as X-ray crystallography, pepscan, phage display, expressed fragments, partial proteolysis, mass spectrometry, and mutagenesis analysis). When talking about protein antigens, many of these methods typically identify linear stretches as epitopes, while epitopes on protein antigens may also be conformational or discontinuous.

The interaction between the binding domain and the target antigen implies that a binding domain exhibits appreciable or significant affinity for the target antigen (here: BCMA and CD3, respectively). In general, it does furthermore not exhibit significant affinity for proteins or antigens other than the target antigen (here: BCMA/CD3)—notwithstanding the above discussed cross-reactivity with homologous targets e.g. from other species. “Significant affinity” includes binding with an affinity (dissociation constant, KD) of ≤10⁻⁶ M. Preferably, binding is considered specific when the binding affinity is ≤10⁻⁷ M, ≤10⁻⁸ M, ≤10⁻⁹ M, ≤10⁻¹⁰ M, or even ≤10⁻¹¹ M, or ≤10⁻¹² M. Whether a binding domain (immuno-) specifically reacts with or binds to a target can be tested readily e.g. by comparing the affinity of said binding domain (or a protein comprising the binding domain) to its desired target protein or antigen with the affinity of said binding domain to non-target proteins or antigens (here: proteins other than BCMA or CD3, respectively). Preferably, a protein of the invention does not significantly bind to targets or antigens other than BCMA or CD3, respectively (i.e., the first domain does not bind to proteins other than BCMA and the second domain does not bind to proteins other than CD3). For example, it is envisaged that the protein of the invention (and more specifically its first domain) does not significantly bind to, interact with, recognize or cross-react with human BAFF-R and/or human TACI.

The binding protein of the invention has an equilibrium dissociation constant (KD) to BCMA which can be determined by Scatchard or by biacore analysis, as described e.g. in WO 2013/072406. The KD values for CD3 can e.g. be determined by surface plasmon resonance analysis, as described e.g. in WO 2013/072406. It is envisaged that the binding protein of the present invention has a KD value for BCMA and/or for CD3 in the 2-digit or 1-digit nanomolar range or in the three digit or even two digit picomolar range.

The term “does not significantly bind” means that a binding domain or a protein comprising the binding domain of the present invention does not bind a protein or antigen other than BCMA or CD3, i.e., shows reactivity of ≤10%, particularly preferably ≤9%, ≤8%, ≤7%, ≤6% or ≤5% with proteins or antigens other than BCMA or CD3, whereby binding to BCMA or CD3, respectively, is set to be 100%.

According to one embodiment of the protein of the present invention, the first and/or the second domain are in the format of an scFv (single-chain variable fragment). An scFv is not actually a fragment of an antibody, but instead is a fusion protein of the variable regions of the heavy (VH) and light chains (VL) of immunoglobulins, connected with a short linker peptide. The linker is usually rich in glycine for flexibility, as well as serine or threonine for solubility. The scFv is designed to retain the specificity of the original immunoglobulin, despite removal of the constant regions and the introduction of the linker. In an scFv, the VH region and the and VL region are arranged in the order VH-VL or VL-VH (from N- to C-terminus). It is envisaged that the VH and the VL regions of the first and/or the second binding domain are connected via a linker, preferably a peptide linker. According to one embodiment of the first and/or the second domain, the VH-region is positioned N-terminally of the linker, and the VL-region is positioned C-terminally of the linker. It is furthermore envisaged that the first domain and the second domain of the protein are connected via a linker, preferably a peptide linker. The linkers are preferably peptide linkers, more preferably short peptide linkers. Examples are shown in SEQ ID NOs: 686-699. In the present context, a “short” linker has between 3 and 25 amino acids, between 4 and 23 amino acids, between 5 and 22 amino acids, between 6 and 20 or between 6 and 17 amino acids. The linker between two variable regions of one binding domain may have a different length (e.g. may be longer) than the linker between the two binding domains. For example, the linker between two variable regions of one binding domain may have a length between 7 and 15 amino acids, preferably between 9 and 13 amino acids, and the linker between the two binding domains may have a length between 3 and 10 amino acids, preferably between 4 and 8. It is further envisaged that the peptide linkers are glycine/serine linkers, such as those depicted in SEQ ID NOs: 687, 689-699.

It is envisaged for the protein of the present invention that
a) the protein is a single chain polypeptide,
b) the first domain is in the format of an scFv,
c) the second domain is in the format of an scFv, and/or
d) the first and the second domain are connected via a linker, preferably a peptide linker, more preferably a glycine/serine linker.

If the protein of the present invention is a single chain polypeptide, it is envisaged that the domains are arranged in the following N-terminal to C-terminal order:
first domain—second domain—third domain (preferred arrangement),
first domain—third domain—second domain,
second domain—first domain—third domain,
second domain—third domain—first domain,
third domain—first domain—second domain, or
third domain—second domain—first domain.

The first domain of the protein of the invention binds to BCMA (B cell maturation antigen, TNFRSF17, CD269). More preferably, it binds to BCMA on the surface of a target cell. The “target cell” can be any prokaryotic or eukaryotic cell expressing BCMA on its surface; preferably the target cell is a cell that is part of the human or animal body, such as a cell of a BCMA positive neoplasm or a BCMA expressing cancer or tumor cell. It is furthermore envisaged that the first domain binds to human BCMA, preferably to human BCMA on the surface of a target cell. It is also envisaged that the first domain binds to macaque BCMA, preferably to macaque BCMA on the surface of a target cell. A preferred amino acid sequence for human BCMA is depicted in SEQ ID NO: 647, for macaque BCMA in SEQ ID NO: 648, for the extracellular domain of human BCMA in SEQ ID NO: 649, for the extracellular domain of macaque BCMA in SEQ ID NO: 650.

In one embodiment of the present invention, the first domain of the protein binds to epitope cluster 3 of BCMA. More preferably, it binds to epitope cluster 3 of human BCMA. A preferred amino acid sequence for epitope cluster 3 of human BCMA is depicted in SEQ ID NO: 651. Proteins having domains that bind to said epitope cluster 3 of BCMA are described in detail in WO 2013/072406 where they are shown to have a very beneficial epitope/activity relationship.

It is furthermore envisaged for the protein of the present invention that the first domain which binds to BCMA comprises a VH region comprising CDR-H1, CDR-H2 and CDR-H3 and a VL region comprising CDR-L1. CDR-L2 and CDR-L3 selected from:
(1) CDR-H1 as depicted in SEQ ID NO: 1. CDR-H2 as depicted in SEQ ID NO: 2, CDR-H3 as depicted in SEQ ID NO: 3, CDR-L1 as depicted in SEQ ID NO: 4, CDR-L2 as depicted in SEQ ID NO: 5, and CDR-L3 as depicted in SEQ ID NO: 6;
(2) CDR-H1 as depicted in SEQ ID NO: 11, CDR-H2 as depicted in SEQ ID NO: 12, CDR-H3 as depicted in SEQ ID NO: 13, CDR-L1 as depicted in SEQ ID NO: 14, CDR-L2 as depicted in SEQ ID NO: 15, and CDR-L3 as depicted in SEQ ID NO: 16;
(3) CDR-H1 as depicted in SEQ ID NO: 21, CDR-H2 as depicted in SEQ ID NO: 22, CDR-H3 as depicted in SEQ ID NO: 23, CDR-L1 as depicted in SEQ ID NO: 24, CDR-L2 as depicted in SEQ ID NO: 25, and CDR-L3 as depicted in SEQ ID NO: 26;
(4) CDR-H1 as depicted in SEQ ID NO: 31, CDR-H2 as depicted in SEQ ID NO: 32, CDR-H3 as depicted in SEQ ID NO: 33, CDR-L1 as depicted in SEQ ID NO: 34, CDR-L2 as depicted in SEQ ID NO: 35, and CDR-L3 as depicted in SEQ ID NO: 36;
(5) CDR-H1 as depicted in SEQ ID NO: 41, CDR-H2 as depicted in SEQ ID NO: 42, CDR-H3 as depicted in SEQ ID NO: 43, CDR-L1 as depicted in SEQ ID NO: 44, CDR-L2 as depicted in SEQ ID NO: 45, and CDR-L3 as depicted in SEQ ID NO: 46;
(6) CDR-H1 as depicted in SEQ ID NO: 51. CDR-H2 as depicted in SEQ ID NO: 52, CDR-H3 as depicted in SEQ ID NO: 53. CDR-L1 as depicted in SEQ ID NO: 54, CDR-L2 as depicted in SEQ ID NO: 55, and CDR-L3 as depicted in SEQ ID NO: 56;
(7) CDR-H1 as depicted in SEQ ID NO: 61, CDR-H2 as depicted in SEQ ID NO: 62, CDR-H3 as depicted in SEQ ID NO: 63, CDR-L1 as depicted in SEQ ID NO: 64. CDR-L2 as depicted in SEQ ID NO: 65, and CDR-L3 as depicted in SEQ ID NO: 66;
(8) CDR-H1 as depicted in SEQ ID NO: 71, CDR-H2 as depicted in SEQ ID NO: 72, CDR-H3 as depicted in SEQ ID NO: 73, CDR-L1 as depicted in SEQ ID NO: 74, CDR-L2 as depicted in SEQ ID NO: 75, and CDR-L3 as depicted in SEQ ID NO: 76;
(9) CDR-H1 as depicted in SEQ ID NO: 81, CDR-H2 as depicted in SEQ ID NO: 82, CDR-H3 as depicted in SEQ ID NO: 83, CDR-L1 as depicted in SEQ ID NO: 84, CDR-L2 as depicted in SEQ ID NO: 85, and CDR-L3 as depicted in SEQ ID NO: 86;
(10) CDR-H1 as depicted in SEQ ID NO: 91. CDR-H2 as depicted in SEQ ID NO: 92, CDR-H3 as depicted in SEQ ID NO: 93, CDR-L1 as depicted in SEQ ID NO: 94, CDR-L2 as depicted in SEQ ID NO: 95, and CDR-L3 as depicted in SEQ ID NO: 96;
(11) CDR-H1 as depicted in SEQ ID NO: 101, CDR-H2 as depicted in SEQ ID NO: 102, CDR-H3 as depicted in SEQ ID NO: 103, CDR-L1 as depicted in SEQ ID NO: 104, CDR-L2 as depicted in SEQ ID NO: 105, and CDR-L3 as depicted in SEQ ID NO: 106;
(12) CDR-H1 as depicted in SEQ ID NO: 111, CDR-H2 as depicted in SEQ ID NO: 112, CDR-H3 as depicted in SEQ ID NO: 113, CDR-L1 as depicted in SEQ ID NO: 114, CDR-L2 as depicted in SEQ ID NO: 115, and CDR-L3 as depicted in SEQ ID NO: 116;
(13) CDR-H1 as depicted in SEQ ID NO: 121, CDR-H2 as depicted in SEQ ID NO: 122, CDR-H3 as depicted in SEQ ID NO: 123, CDR-L1 as depicted in SEQ ID NO: 124, CDR-L2 as depicted in SEQ ID NO: 125, and CDR-L3 as depicted in SEQ ID NO: 126;
(14) CDR-H1 as depicted in SEQ ID NO: 131, CDR-H2 as depicted in SEQ ID NO: 132, CDR-H3 as depicted in SEQ ID NO: 133, CDR-L1 as depicted in SEQ ID NO: 134, CDR-L2 as depicted in SEQ ID NO: 135, and CDR-L3 as depicted in SEQ ID NO: 136;
(15) CDR-H1 as depicted in SEQ ID NO: 141, CDR-H2 as depicted in SEQ ID NO: 142, CDR-H3 as depicted in SEQ ID NO: 143, CDR-L1 as depicted in SEQ ID NO: 144, CDR-L2 as depicted in SEQ ID NO: 145, and CDR-L3 as depicted in SEQ ID NO: 146;
(16) CDR-H1 as depicted in SEQ ID NO: 151, CDR-H2 as depicted in SEQ ID NO: 152, CDR-H3 as depicted in SEQ ID NO: 153, CDR-L1 as depicted in SEQ ID NO: 154, CDR-L2 as depicted in SEQ ID NO: 155, and CDR-L3 as depicted in SEQ ID NO: 156;
(17) CDR-H1 as depicted in SEQ ID NO: 161, CDR-H2 as depicted in SEQ ID NO: 162, CDR-H3 as depicted in SEQ ID NO: 163. CDR-L1 as depicted in SEQ ID NO: 164, CDR-L2 as depicted in SEQ ID NO: 165, and CDR-L3 as depicted in SEQ ID NO: 166;
(18) CDR-H1 as depicted in SEQ ID NO: 171, CDR-H2 as depicted in SEQ ID NO: 172, CDR-H3 as depicted in SEQ ID NO: 173, CDR-L1 as depicted in SEQ ID NO: 174, CDR-L2 as depicted in SEQ ID NO: 175, and CDR-L3 as depicted in SEQ ID NO: 176;
(19) CDR-H1 as depicted in SEQ ID NO: 181, CDR-H2 as depicted in SEQ ID NO: 182, CDR-H3 as depicted in SEQ ID NO: 183, CDR-L1 as depicted in SEQ ID NO: 184, CDR-L2 as depicted in SEQ ID NO: 185, and CDR-L3 as depicted in SEQ ID NO: 186;
(20) CDR-H1 as depicted in SEQ ID NO: 191, CDR-H2 as depicted in SEQ ID NO: 192, CDR-H3 as depicted in SEQ ID NO: 193, CDR-L1 as depicted in SEQ ID NO: 194, CDR-L2 as depicted in SEQ ID NO: 195, and CDR-L3 as depicted in SEQ ID NO: 196;
(21) CDR-H1 as depicted in SEQ ID NO: 201, CDR-H2 as depicted in SEQ ID NO: 202, CDR-H3 as depicted in SEQ ID NO: 203, CDR-L1 as depicted in SEQ ID NO: 204, CDR-L2 as depicted in SEQ ID NO: 205, and CDR-L3 as depicted in SEQ ID NO: 206;
(22) CDR-H1 as depicted in SEQ ID NO: 211, CDR-H2 as depicted in SEQ ID NO: 212, CDR-H3 as depicted in SEQ ID NO: 213, CDR-L1 as depicted in SEQ ID NO: 214, CDR-L2 as depicted in SEQ ID NO: 215, and CDR-L3 as depicted in SEQ ID NO: 216;
(23) CDR-H1 as depicted in SEQ ID NO: 221, CDR-H2 as depicted in SEQ ID NO: 222, CDR-H3 as depicted in SEQ ID NO: 223, CDR-L1 as depicted in SEQ ID NO: 224, CDR-L2 as depicted in SEQ ID NO: 225, and CDR-L3 as depicted in SEQ ID NO: 226;
(24) CDR-H1 as depicted in SEQ ID NO: 231, CDR-H2 as depicted in SEQ ID NO: 232, CDR-H3 as depicted in SEQ ID NO: 233, CDR-L1 as depicted in SEQ ID NO: 234, CDR-L2 as depicted in SEQ ID NO: 235, and CDR-L3 as depicted in SEQ ID NO: 236;
(25) CDR-H1 as depicted in SEQ ID NO: 241. CDR-H2 as depicted in SEQ ID NO: 242, CDR-H3 as depicted in SEQ ID NO: 243, CDR-L1 as depicted in SEQ ID NO: 244, CDR-L2 as depicted in SEQ ID NO: 245, and CDR-L3 as depicted in SEQ ID NO: 246;
(26) CDR-H1 as depicted in SEQ ID NO: 251, CDR-H2 as depicted in SEQ ID NO: 252, CDR-H3 as depicted in SEQ ID NO: 253, CDR-L1 as depicted in SEQ ID NO: 254, CDR-L2 as depicted in SEQ ID NO: 255, and CDR-L3 as depicted in SEQ ID NO: 256;
(27) CDR-H1 as depicted in SEQ ID NO: 261, CDR-H2 as depicted in SEQ ID NO: 262, CDR-H3 as depicted in SEQ ID NO: 263, CDR-L1 as depicted in SEQ ID NO: 264, CDR-L2 as depicted in SEQ ID NO: 265, and CDR-L3 as depicted in SEQ ID NO: 266;
(28) CDR-H1 as depicted in SEQ ID NO: 271, CDR-H2 as depicted in SEQ ID NO: 272, CDR-H3 as depicted in SEQ ID NO: 273, CDR-L1 as depicted in SEQ ID NO: 274, CDR-L2 as depicted in SEQ ID NO: 275, and CDR-L3 as depicted in SEQ ID NO: 276;
(29) CDR-H1 as depicted in SEQ ID NO: 281, CDR-H2 as depicted in SEQ ID NO: 282, CDR-H3 as depicted in SEQ ID NO: 283, CDR-L1 as depicted in SEQ ID NO: 284, CDR-L2 as depicted in SEQ ID NO: 285, and CDR-L3 as depicted in SEQ ID NO: 286;
(30) CDR-H1 as depicted in SEQ ID NO: 291. CDR-H2 as depicted in SEQ ID NO: 292, CDR-H3 as depicted in SEQ ID NO: 293, CDR-L1 as depicted in SEQ ID NO: 294, CDR-L2 as depicted in SEQ ID NO: 295, and CDR-L3 as depicted in SEQ ID NO: 296;
(31) CDR-H1 as depicted in SEQ ID NO: 301, CDR-H2 as depicted in SEQ ID NO: 302, CDR-H3 as depicted in SEQ ID NO: 303, CDR-L1 as depicted in SEQ ID NO: 304, CDR-L2 as depicted in SEQ ID NO: 305, and CDR-L3 as depicted in SEQ ID NO: 306;
(32) CDR-H1 as depicted in SEQ ID NO: 311, CDR-H2 as depicted in SEQ ID NO: 312, CDR-H3 as depicted in SEQ ID NO: 313, CDR-L1 as depicted in SEQ ID NO: 314, CDR-L2 as depicted in SEQ ID NO: 315, and CDR-L3 as depicted in SEQ ID NO: 316;
(33) CDR-H1 as depicted in SEQ ID NO: 321, CDR-H2 as depicted in SEQ ID NO: 322, CDR-H3 as depicted in SEQ ID NO: 323, CDR-L1 as depicted in SEQ ID NO: 324, CDR-L2 as depicted in SEQ ID NO: 325, and CDR-L3 as depicted in SEQ ID NO: 326;
(34) CDR-H1 as depicted in SEQ ID NO: 331, CDR-H2 as depicted in SEQ ID NO: 332, CDR-H3 as depicted in SEQ ID NO: 333, CDR-L1 as depicted in SEQ ID NO: 334, CDR-L2 as depicted in SEQ ID NO: 335, and CDR-L3 as depicted in SEQ ID NO: 336;
(35) CDR-H1 as depicted in SEQ ID NO: 341, CDR-H2 as depicted in SEQ ID NO: 342, CDR-H3 as depicted in SEQ ID NO: 343, CDR-L1 as depicted in SEQ ID NO: 344, CDR-L2 as depicted in SEQ ID NO: 345, and CDR-L3 as depicted in SEQ ID NO: 346;
(36) CDR-H1 as depicted in SEQ ID NO: 351, CDR-H2 as depicted in SEQ ID NO: 352, CDR-H3 as depicted in SEQ ID NO: 353, CDR-L1 as depicted in SEQ ID NO: 354, CDR-L2 as depicted in SEQ ID NO: 355, and CDR-L3 as depicted in SEQ ID NO: 356;
(37) CDR-H1 as depicted in SEQ ID NO: 361, CDR-H2 as depicted in SEQ ID NO: 362, CDR-H3 as depicted in SEQ ID NO: 363, CDR-L1 as depicted in SEQ ID NO: 364, CDR-L2 as depicted in SEQ ID NO: 365, and CDR-L3 as depicted in SEQ ID NO: 366;
(38) CDR-H1 as depicted in SEQ ID NO: 371, CDR-H2 as depicted in SEQ ID NO: 372, CDR-H3 as depicted in SEQ ID NO: 373, CDR-L1 as depicted in SEQ ID NO: 374, CDR-L2 as depicted in SEQ ID NO: 375, and CDR-L3 as depicted in SEQ ID NO: 376;
(39) CDR-H1 as depicted in SEQ ID NO: 381, CDR-H2 as depicted in SEQ ID NO: 382, CDR-H3 as depicted in SEQ ID NO: 383, CDR-L1 as depicted in SEQ ID NO: 384, CDR-L2 as depicted in SEQ ID NO: 385, and CDR-L3 as depicted in SEQ ID NO: 386;
(40) CDR-H1 as depicted in SEQ ID NO: 391, CDR-H2 as depicted in SEQ ID NO: 392, CDR-H3 as depicted in SEQ ID NO: 393, CDR-L1 as depicted in SEQ ID NO: 394, CDR-L2 as depicted in SEQ ID NO: 395, and CDR-L3 as depicted in SEQ ID NO: 396;
(41) CDR-H1 as depicted in SEQ ID NO: 401, CDR-H2 as depicted in SEQ ID NO: 402, CDR-H3 as depicted in SEQ ID NO: 403, CDR-L1 as depicted in SEQ ID NO: 404, CDR-L2 as depicted in SEQ ID NO: 405, and CDR-L3 as depicted in SEQ ID NO: 406;
(42) CDR-H1 as depicted in SEQ ID NO: 411. CDR-H2 as depicted in SEQ ID NO: 412, CDR-H3 as depicted in SEQ ID NO: 413, CDR-L1 as depicted in SEQ ID NO: 414, CDR-L2 as depicted in SEQ ID NO: 415, and CDR-L3 as depicted in SEQ ID NO: 416;
(43) CDR-H1 as depicted in SEQ ID NO: 421, CDR-H2 as depicted in SEQ ID NO: 422, CDR-H3 as depicted in SEQ ID NO: 423, CDR-L1 as depicted in SEQ ID NO: 424, CDR-L2 as depicted in SEQ ID NO: 425, and CDR-L3 as depicted in SEQ ID NO: 426;
(44) CDR-H1 as depicted in SEQ ID NO: 431, CDR-H2 as depicted in SEQ ID NO: 432, CDR-H3 as depicted in SEQ ID NO: 433, CDR-L1 as depicted in SEQ ID NO: 434, CDR-L2 as depicted in SEQ ID NO: 435, and CDR-L3 as depicted in SEQ ID NO: 436;
(45) CDR-H1 as depicted in SEQ ID NO: 441, CDR-H2 as depicted in SEQ ID NO: 442, CDR-H3 as depicted in SEQ ID NO: 443, CDR-L1 as depicted in SEQ ID NO: 444, CDR-L2 as depicted in SEQ ID NO: 445, and CDR-L3 as depicted in SEQ ID NO: 446;
(46) CDR-H1 as depicted in SEQ ID NO: 451, CDR-H2 as depicted in SEQ ID NO: 452, CDR-H3 as depicted in SEQ ID NO: 453, CDR-L1 as depicted in SEQ ID NO: 454, CDR-L2 as depicted in SEQ ID NO: 455, and CDR-L3 as depicted in SEQ ID NO: 456;
(47) CDR-H1 as depicted in SEQ ID NO: 461. CDR-H2 as depicted in SEQ ID NO: 462, CDR-H3 as depicted in SEQ ID NO: 463, CDR-L1 as depicted in SEQ ID NO: 464, CDR-L2 as depicted in SEQ ID NO: 465, and CDR-L3 as depicted in SEQ ID NO: 466;
(48) CDR-H1 as depicted in SEQ ID NO: 471, CDR-H2 as depicted in SEQ ID NO: 472, CDR-H3 as depicted in SEQ ID NO: 473, CDR-L1 as depicted in SEQ ID NO: 474, CDR-L2 as depicted in SEQ ID NO: 475, and CDR-L3 as depicted in SEQ ID NO: 476;
(49) CDR-H1 as depicted in SEQ ID NO: 481, CDR-H2 as depicted in SEQ ID NO: 482, CDR-H3 as depicted in SEQ ID NO: 483, CDR-L1 as depicted in SEQ ID NO: 484, CDR-L2 as depicted in SEQ ID NO: 485, and CDR-L3 as depicted in SEQ ID NO: 486;
(50) CDR-H1 as depicted in SEQ ID NO: 491, CDR-H2 as depicted in SEQ ID NO: 492, CDR-H3 as depicted in SEQ ID NO: 493, CDR-L1 as depicted in SEQ ID NO: 494, CDR-L2 as depicted in SEQ ID NO: 495, and CDR-L3 as depicted in SEQ ID NO: 496;
(51) CDR-H1 as depicted in SEQ ID NO: 501, CDR-H2 as depicted in SEQ ID NO: 502, CDR-H3 as depicted in SEQ ID NO: 503, CDR-L1 as depicted in SEQ ID NO: 504, CDR-L2 as depicted in SEQ ID NO: 505, and CDR-L3 as depicted in SEQ ID NO: 506;
(52) CDR-H1 as depicted in SEQ ID NO: 511, CDR-H2 as depicted in SEQ ID NO: 512, CDR-H3 as depicted in SEQ ID NO: 513, CDR-L1 as depicted in SEQ ID NO: 514, CDR-L2 as depicted in SEQ ID NO: 515, and CDR-L3 as depicted in SEQ ID NO: 516; and
(53) CDR-H1 as depicted in SEQ ID NO: 521, CDR-H2 as depicted in SEQ ID NO: 522, CDR-H3 as depicted in SEQ ID NO: 523, CDR-L1 as depicted in SEQ ID NO: 524, CDR-L2 as depicted in SEQ ID NO: 525, and CDR-L3 as depicted in SEQ ID NO: 526.

It is also envisaged for the protein of the present invention that the first domain which binds to BCMA comprises a VL region having an amino acid sequence selected from the group consisting of those depicted in SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, and 528. It is envisaged that the first domain comprises a VL region having an amino acid sequence as depicted in SEQ ID NO: 178.

It is furthermore envisaged that the first domain which binds to BCMA comprises a VH region having an amino acid sequence selected from the group consisting of those depicted in SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 307, 317, 327, 337, 347, 357, 367, 377, 387, 397, 407, 417, 427, 437, 447, 457, 467, 477, 487, 497, 507, 517, and 527. It is envisaged that the first domain comprises a VH region having an amino acid sequence as depicted in SEQ ID NO: 177.

It is also envisaged for the protein of the present invention that the first domain which binds to BCMA comprises a VH region and a VL region selected from the group consisting of:
(1) a VH region as depicted in SEQ ID NO: 7 and a VL region as depicted in SEQ ID NO: 8;
(2) a VH region as depicted in SEQ ID NO: 17 and a VL region as depicted in SEQ ID NO: 18;
(3) a VH region as depicted in SEQ ID NO: 27 and a VL region as depicted in SEQ ID NO: 28;
(4) a VH region as depicted in SEQ ID NO: 37 and a VL region as depicted in SEQ ID NO: 38;
(5) a VH region as depicted in SEQ ID NO: 47 and a VL region as depicted in SEQ ID NO: 48;
(6) a VH region as depicted in SEQ ID NO: 57 and a VL region as depicted in SEQ ID NO: 58;
(7) a VH region as depicted in SEQ ID NO: 67 and a VL region as depicted in SEQ ID NO: 68;
(8) a VH region as depicted in SEQ ID NO: 77 and a VL region as depicted in SEQ ID NO: 78;
(9) a VH region as depicted in SEQ ID NO: 87 and a VL region as depicted in SEQ ID NO: 88;
(10) a VH region as depicted in SEQ ID NO: 97 and a VL region as depicted in SEQ ID NO: 98;
(11) a VH region as depicted in SEQ ID NO: 107 and a VL region as depicted in SEQ ID NO: 108;
(12) a VH region as depicted in SEQ ID NO: 117 and a VL region as depicted in SEQ ID NO: 118;
(13) a VH region as depicted in SEQ ID NO: 127 and a VL region as depicted in SEQ ID NO: 128;
(14) a VH region as depicted in SEQ ID NO: 137 and a VL region as depicted in SEQ ID NO: 138;
(15) a VH region as depicted in SEQ ID NO: 147 and a VL region as depicted in SEQ ID NO: 148;
(16) a VH region as depicted in SEQ ID NO: 157 and a VL region as depicted in SEQ ID NO: 158;
(17) a VH region as depicted in SEQ ID NO: 167 and a VL region as depicted in SEQ ID NO: 168;
(18) a VH region as depicted in SEQ ID NO: 177 and a VL region as depicted in SEQ ID NO: 178;
(19) a VH region as depicted in SEQ ID NO: 187 and a VL region as depicted in SEQ ID NO: 188;
(20) a VH region as depicted in SEQ ID NO: 197 and a VL region as depicted in SEQ ID NO: 198;
(21) a VH region as depicted in SEQ ID NO: 207 and a VL region as depicted in SEQ ID NO: 208;
(22) a VH region as depicted in SEQ ID NO: 217 and a VL region as depicted in SEQ ID NO: 218;
(23) a VH region as depicted in SEQ ID NO: 227 and a VL region as depicted in SEQ ID NO: 228;
(24) a VH region as depicted in SEQ ID NO: 237 and a VL region as depicted in SEQ ID NO: 238;
(25) a VH region as depicted in SEQ ID NO: 247 and a VL region as depicted in SEQ ID NO: 248;
(26) a VH region as depicted in SEQ ID NO: 257 and a VL region as depicted in SEQ ID NO: 258;
(27) a VH region as depicted in SEQ ID NO: 267 and a VL region as depicted in SEQ ID NO: 268;
(28) a VH region as depicted in SEQ ID NO: 277 and a VL region as depicted in SEQ ID NO: 278;
(29) a VH region as depicted in SEQ ID NO: 287 and a VL region as depicted in SEQ ID NO: 288;
(30) a VH region as depicted in SEQ ID NO: 297 and a VL region as depicted in SEQ ID NO: 298;
(31) a VH region as depicted in SEQ ID NO: 307 and a VL region as depicted in SEQ ID NO: 308;
(32) a VH region as depicted in SEQ ID NO: 317 and a VL region as depicted in SEQ ID NO: 318;
(33) a VH region as depicted in SEQ ID NO: 327 and a VL region as depicted in SEQ ID NO: 328;
(34) a VH region as depicted in SEQ ID NO: 337 and a VL region as depicted in SEQ ID NO: 338;
(35) a VH region as depicted in SEQ ID NO: 347 and a VL region as depicted in SEQ ID NO: 348;
(36) a VH region as depicted in SEQ ID NO: 357 and a VL region as depicted in SEQ ID NO: 358;
(37) a VH region as depicted in SEQ ID NO: 367 and a VL region as depicted in SEQ ID NO: 368;
(38) a VH region as depicted in SEQ ID NO: 377 and a VL region as depicted in SEQ ID NO: 378;
(39) a VH region as depicted in SEQ ID NO: 387 and a VL region as depicted in SEQ ID NO: 388;
(40) a VH region as depicted in SEQ ID NO: 397 and a VL region as depicted in SEQ ID NO: 398;
(41) a VH region as depicted in SEQ ID NO: 407 and a VL region as depicted in SEQ ID NO: 408;
(42) a VH region as depicted in SEQ ID NO: 417 and a VL region as depicted in SEQ ID NO: 418;
(43) a VH region as depicted in SEQ ID NO: 427 and a VL region as depicted in SEQ ID NO: 428;
(44) a VH region as depicted in SEQ ID NO: 437 and a VL region as depicted in SEQ ID NO: 438;
(45) a VH region as depicted in SEQ ID NO: 447 and a VL region as depicted in SEQ ID NO: 448;
(46) a VH region as depicted in SEQ ID NO: 457 and a VL region as depicted in SEQ ID NO: 458;
(47) a VH region as depicted in SEQ ID NO: 467 and a VL region as depicted in SEQ ID NO: 468;
(48) a VH region as depicted in SEQ ID NO: 477 and a VL region as depicted in SEQ ID NO: 478;
(49) a VH region as depicted in SEQ ID NO: 487 and a VL region as depicted in SEQ ID NO: 488;
(50) a VH region as depicted in SEQ ID NO: 497 and a VL region as depicted in SEQ ID NO: 498;
(51) a VH region as depicted in SEQ ID NO: 507 and a VL region as depicted in SEQ ID NO: 508;
(52) a VH region as depicted in SEQ ID NO: 517 and a VL region as depicted in SEQ ID NO: 518; and
(53) a VH region as depicted in SEQ ID NO: 527 and a VL region as depicted in SEQ ID NO: 528.

It is envisaged that the first domain comprises a VH region having an amino acid sequence as depicted in SEQ ID NO: 177 and a VL region having an amino acid sequence as depicted in SEQ ID NO: 178.

It is also envisaged for the protein of the present invention that the first domain which binds to BCMA comprises a polypeptide having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529. It is envisaged that the first domain comprises a polypeptide having an amino acid sequence as depicted in SEQ ID NO: 179.

“T cells” or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different polypeptide chains. In 95% of the T cells, the TCR consists of an alpha (a) and beta (B) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

“CD3” (cluster of differentiation 3) is a T cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ζ (zeta) chain to form the “T cell receptor complex” and to generate an activation signal in T lymphocytes. The CD3γ (gamma), CD38 (delta), and CD38 (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. CD3 epsilon is a polypeptide which in humans is encoded by the CD3E gene which resides on chromosome 11.

The redirected lysis of target cells via the recruitment of T cells by a binding protein which binds to CD3 on the T cell and to a target protein (here: BCMA) on the target cell generally involves cytolytic synapse formation and delivery of perforin and granzymes. The engaged T cells are capable of serial target cell lysis and are not affected by immune escape mechanisms interfering with peptide antigen processing and presentation, or clonal T cell differentiation; see, for example, WO 2007/042261.

Cytotoxicity mediated by anti-BCMA x anti-CD3 binding proteins can be measured in various ways. The “half maximal effective concentration” (EC₅₀) is commonly used as a measure of potency of a biologically active molecule such as a protein of the present invention. It is expressed in molar units. In the present case of measuring cytotoxicity, the EC₅₀ value refers to the concentration of a binding protein inducing a cytotoxic response (lysis of target cells) halfway between the baseline and the maximum. Effector cells in a cytotoxicity assay can e.g. be stimulated enriched (human) CD8 positive T cells or unstimulated (human) peripheral blood mononuclear cells (PBMC). The target cells should express BCMA on the cell surface. Preferably the target cells express (at least) the extracellular domain of BCMA on the cell surface. Target cells can be cells from a cell line (such as CHO) which is stably or transiently transfected with BCMA, e.g. human BCMA. Alternatively, the target cells can be cells from a BCMA positive natural expresser cell line, such as the human multiple myeloma cell line L363 or NCI-H929.

The effector to target cell (E:T) ratio in a cytotoxicity assay is usually about 10:1, but can also vary. Cytotoxic activity of an anti-BCMA x anti-CD3 binding protein can be measured in a 51-chromium release assay (e.g. with an incubation time of about 18 hours) or in a in a FACS-based cytotoxicity assay (e.g. with an incubation time of about 48 hours). Modifications of the incubation time (cytotoxic reaction) are also envisaged.

According to one embodiment, the cytotoxic activity mediated by an anti-BCMA x anti-CD3 binding protein of the present invention is measured in a FACS-based cytotoxicity assay. The EC₅₀ value may be ≤1000 pM, ≤900 pM, ≤800 pM, ≤700 pM, ≤600 pM, ≤500 pM, ≤400 pM, ≤300 pM, ≤200 pM, ≤100 pM, ≤90 pM, ≤80 pM, ≤70 pM, ≤60 pM, ≤50 pM, ≤40 pM, ≤30 pM, ≤20 pM, ≤10 pM, or ≤5 pM. It is also envisaged that the binding protein of the present invention has an EC₅₀ value in the 3-digit to 1-digit pM range, as determined in a FACS-based cytotoxicity assay. The assay may be carried out with the L363 or NCI-H929 cell line or with BCMA transfected CHO cells as target cells and stimulated enriched (human) CD8 positive T cells or unstimulated (human) PBMC as effector cells. See also WO 2013/072406.

The second domain of the protein of the invention binds to CD3. More preferably, it binds to CD3 on the surface of a T cell. It is furthermore envisaged that the second domain binds to human CD3, preferably to human CD3 on the surface of a T cell. It is also envisaged that the second domain binds to CD3 epsilon. More preferably, it binds to human CD3 epsilon, e.g. to human CD3 epsilon on the surface of a T cell. A preferred amino acid sequence for the extracellular domain of human CD3 epsilon is depicted in SEQ ID NO: 653.

In one embodiment of the present invention, the second domain of the protein binds to human CD3 epsilon (or human CD3 epsilon on the surface of a T cell) and to Callithrix jacchus or Saimiri sciureus CD3 epsilon. It is also envisaged that the second domain binds to an extracellular epitope of CD3 epsilon, preferably to an extracellular epitope of human CD3 epsilon. It is also envisaged that the second domain binds to an extracellular epitope of the human and the Macaca CD3 epsilon chain. One preferred epitope of CD3 epsilon is comprised within amino acid residues 1-27 of the human CD3 epsilon extracellular domain (see SEQ ID NO: 654). Even more specifically, the epitope comprises at least the amino acid sequence Gln-Asp-Gly-Asn-Glu. Callithrix jacchus is a new world primate belonging to the family of Callitrichidae, while Saimiri sciureus is a new world primate belonging to the family of Cebidae. Binders having such characteristics are described in detail in WO 2008/119567.

Antibodies or (bispecific) binding proteins directed against (human) CD3 or specifically against CD3 epsilon are known in the art, and their CDRs, VH and VL sequences can serve as a basis for the second binding domain of the binding protein of the invention. For example, Kung et al. reported in 1979 the development of OKT3 (Ortho Kung T3), the first mAb recognizing CD3 (specifically, the epsilon chain of CD3) on human T cells. OKT3 (muromonab) was the first monoclonal antibody of murine origin to become available for therapy in humans. Newer anti-CD3 monoclonal antibodies include otelixizumab (TRX4), teplizumab (MGA031), foralumab and visilizumab, all targeting the epsilon chain of CD3. Bispecific binding proteins directed against a (cancer) target and CD3 are also being developed and (pre-) clinically tested, and their CD3 binding domain (CDRs, VH, VL) may serve as a basis for the second binding domain of the binding protein of the invention. Examples include, but are not limited to, Blinatumomab, Solitomab (MT110, AMG 110), Catumaxomab, Duvortuxizumab, Ertumaxomab, Mosunetuzumab, FBTA05 (Bi20, TPBs05), CEA-TCB (RG7802, RO6958688), AFM11, and MGD006 (S80880). Other examples of CD3 binding domains are disclosed e.g. in U.S. Pat. No. 7,994,289 B2, U.S. Pat. No. 7,728,114 B2, U.S. Pat. No. 7,381,803 B1, U.S. Pat. No. 6,706,265 B1.

It is envisaged for the protein of the present invention that the second domain which binds to CD3 comprises a VL region comprising CDR-L1, CDR-L2 and CDR-L3 selected from:
(a) CDR-L1 as depicted in SEQ ID NO: 542, CDR-L2 as depicted in SEQ ID NO: 543, and CDR-L3 as depicted in SEQ ID NO: 544;
(b) CDR-L1 as depicted in SEQ ID NO: 599, CDR-L2 as depicted in SEQ ID NO: 600, and CDR-L3 as depicted in SEQ ID NO: 601; and
(c) CDR-L1 as depicted in SEQ ID NO: 621, CDR-L2 as depicted in SEQ ID NO: 622, and CDR-L3 as depicted in SEQ ID NO: 623.

It is also envisaged for the protein of the present invention that the second domain which binds to CD3 comprises a VH region comprising CDR-H1, CDR-H2 and CDR-H3 selected from:
(a) CDR-H1 as depicted in SEQ ID NO: 534, CDR-H2 as depicted in SEQ ID NO: 535, and CDR-H3 as depicted in SEQ ID NO: 536;
(b) CDR-H1 as depicted in SEQ ID NO: 545, CDR-H2 as depicted in SEQ ID NO: 546, and CDR-H3 as depicted in SEQ ID NO: 547;
(c) CDR-H1 as depicted in SEQ ID NO: 557, CDR-H2 as depicted in SEQ ID NO: 558, and CDR-H3 as depicted in SEQ ID NO: 559;
(d) CDR-H1 as depicted in SEQ ID NO: 568, CDR-H2 as depicted in SEQ ID NO: 569, and CDR-H3 as depicted in SEQ ID NO: 570;
(e) CDR-H1 as depicted in SEQ ID NO: 579, CDR-H2 as depicted in SEQ ID NO: 580, and CDR-H3 as depicted in SEQ ID NO: 581;
(f) CDR-H1 as depicted in SEQ ID NO: 591, CDR-H2 as depicted in SEQ ID NO: 592, and CDR-H3 as depicted in SEQ ID NO: 593;
(g) CDR-H1 as depicted in SEQ ID NO: 602, CDR-H2 as depicted in SEQ ID NO: 603, and CDR-H3 as depicted in SEQ ID NO: 604;
(h) CDR-H1 as depicted in SEQ ID NO: 613, CDR-H2 as depicted in SEQ ID NO: 614, and CDR-H3 as depicted in SEQ ID NO: 615;
(i) CDR-H1 as depicted in SEQ ID NO: 624, CDR-H2 as depicted in SEQ ID NO: 625, and CDR-H3 as depicted in SEQ ID NO: 626; and
(j) CDR-H1 as depicted in SEQ ID NO: 636, CDR-H2 as depicted in SEQ ID NO: 637, and CDR-H3 as depicted in SEQ ID NO: 638.

It is furthermore envisaged for the protein of the present invention that the second domain which binds to CD3 comprises a VL region comprising CDR-L1, CDR-L2 and CDR-L3 and a VH region comprising CDR-H1, CDR-H2 and CDR-H3 selected from:
(a) CDR-L1 as depicted in SEQ ID NO: 531, CDR-L2 as depicted in SEQ ID NO: 532, CDR-L3 as depicted in SEQ ID NO: 533, CDR-H1 as depicted in SEQ ID NO: 534, CDR-H2 as depicted in SEQ ID NO: 535, and CDR-H3 as depicted in SEQ ID NO: 536;
(b) CDR-L1 as depicted in SEQ ID NO: 542. CDR-L2 as depicted in SEQ ID NO: 543, CDR-L3 as depicted in SEQ ID NO: 544, CDR-H1 as depicted in SEQ ID NO: 545, CDR-H2 as depicted in SEQ ID NO: 546, and CDR-H3 as depicted in SEQ ID NO: 547;
(c) CDR-L1 as depicted in SEQ ID NO: 554, CDR-L2 as depicted in SEQ ID NO: 555, CDR-L3 as depicted in SEQ ID NO: 556, CDR-H1 as depicted in SEQ ID NO: 557, CDR-H2 as depicted in SEQ ID NO: 558, and CDR-H3 as depicted in SEQ ID NO: 559;
(d) CDR-L1 as depicted in SEQ ID NO: 565, CDR-L2 as depicted in SEQ ID NO: 566, CDR-L3 as depicted in SEQ ID NO: 567, CDR-H1 as depicted in SEQ ID NO: 568, CDR-H2 as depicted in SEQ ID NO: 569, and CDR-H3 as depicted in SEQ ID NO: 570;
(c) CDR-L1 as depicted in SEQ ID NO: 576, CDR-L2 as depicted in SEQ ID NO: 577, CDR-L3 as depicted in SEQ ID NO: 578, CDR-H1 as depicted in SEQ ID NO: 579, CDR-H2 as depicted in SEQ ID NO: 580, and CDR-H3 as depicted in SEQ ID NO: 581;
(f) CDR-L1 as depicted in SEQ ID NO: 588, CDR-L2 as depicted in SEQ ID NO: 589, CDR-L3 as depicted in SEQ ID NO: 590, CDR-H1 as depicted in SEQ ID NO: 591, CDR-H2 as depicted in SEQ ID NO: 592, and CDR-H3 as depicted in SEQ ID NO: 593;
(g) CDR-L1 as depicted in SEQ ID NO: 599, CDR-L2 as depicted in SEQ ID NO: 600, CDR-L3 as depicted in SEQ ID NO: 601. CDR-H1 as depicted in SEQ ID NO: 602, CDR-H2 as depicted in SEQ ID NO: 603, and CDR-H3 as depicted in SEQ ID NO: 604;
(h) CDR-L1 as depicted in SEQ ID NO: 610, CDR-L2 as depicted in SEQ ID NO: 611, CDR-L3 as depicted in SEQ ID NO: 612, CDR-H1 as depicted in SEQ ID NO: 613, CDR-H2 as depicted in SEQ ID NO: 614, and CDR-H3 as depicted in SEQ ID NO: 615;
(i) CDR-L1 as depicted in SEQ ID NO: 621, CDR-L2 as depicted in SEQ ID NO: 622, CDR-L3 as depicted in SEQ ID NO: 623, CDR-H1 as depicted in SEQ ID NO: 624, CDR-H2 as depicted in SEQ ID NO: 625, and CDR-H3 as depicted in SEQ ID NO: 626; and
(j) CDR-L1 as depicted in SEQ ID NO: 633, CDR-L2 as depicted in SEQ ID NO: 634, CDR-L3 as depicted in SEQ ID NO: 635, CDR-H1 as depicted in SEQ ID NO: 636, CDR-H2 as depicted in SEQ ID NO: 637, and CDR-H3 as depicted in SEQ ID NO: 638.

It is also envisaged for the protein of the present invention that the second domain which binds to CD3 comprises a VL region having an amino acid sequence selected from the group consisting of those depicted in SEQ ID NO: 550, SEQ ID NO: 551, SEQ ID NO: 584, SEQ ID NO: 585, SEQ ID NO: 629 and SEQ ID NO: 630, preferably SEQ ID NO: 629.

It is furthermore envisaged that the second domain which binds to CD3 comprises a VH region having an amino acid sequence selected from the group consisting of those depicted in SEQ ID NO: 537, SEQ ID NO: 538, SEQ ID NO: 548, SEQ ID NO: 549, SEQ ID NO: 560, SEQ ID NO: 561, SEQ ID NO: 571, SEQ ID NO: 572, SEQ ID NO: 582, SEQ ID NO: 583, SEQ ID NO: 594, SEQ ID NO: 595, SEQ ID NO: 605, SEQ ID NO: 606, SEQ ID NO: 616, SEQ ID NO: 617, SEQ ID NO: 627, SEQ ID NO: 628, SEQ ID NO: 639, SEQ ID NO: 640, and SEQ ID NO: 644, preferably SEQ ID NO: 639.

It is also envisaged for the protein of the present invention that the second domain which binds to CD3 comprises a VL region and a VH region selected from the group consisting of:
(a) a VL region as depicted in SEQ ID NO: 539 or 521 and a VH region as depicted in SEQ ID NO: 537 or 538;
(b) a VL region as depicted in SEQ ID NO: 550 or 521 and a VH region as depicted in SEQ ID NO: 548 or 549;
(c) a VL region as depicted in SEQ ID NO: 562 or 521 and a VH region as depicted in SEQ ID NO: 560 or 561;
(d) a VL region as depicted in SEQ ID NO: 573 or 521 and a VH region as depicted in SEQ ID NO: 571 or 572;
(e) a VL region as depicted in SEQ ID NO: 584 or 585 and a VH region as depicted in SEQ ID NO: 582 or 583;
(f) a VL region as depicted in SEQ ID NO: 596 or 521 and a VH region as depicted in SEQ ID NO: 594 or 595;
(g) a VL region as depicted in SEQ ID NO: 607 or 585 and a VH region as depicted in SEQ ID NO: 605 or 606;
(h) a VL region as depicted in SEQ ID NO: 618 or 521 and a VH region as depicted in SEQ ID NO: 616 or 617;
(i) a VL region as depicted in SEQ ID NO: 629 or 630 and a VH region as depicted in SEQ ID NO: 627 or 628;
(j) a VL region as depicted in SEQ ID NO: 641 or 630 and a VH region as depicted in SEQ ID NO: 639 or 640; and
(k) a VL region as depicted in SEQ ID NO: 645 and a VH region as depicted in SEQ ID NO: 644.

It is also envisaged for the protein of the present invention that the second domain which binds to CD3 comprises or consists of a polypeptide having an amino acid sequence selected from the group consisting of those depicted in SEQ ID NOs: 540, 541, 552, 553, 563, 564, 574, 575, 586, 587, 597, 598, 608, 609, 619, 620, 631, 632, 642, 643, and 646, preferably SEQ ID NO: 642.

It is also envisaged that the protein of the present invention competes for binding to CD3 with:
a) an antibody or protein comprising a domain which binds to CD3 on the surface of a T cell, wherein said domain comprises a VH region comprising CDR-H1 as depicted in SEQ ID NO: 636, CDR-H2 as depicted in SEQ ID NO: 637, and CDR-H3 as depicted in SEQ ID NO: 638, and a VL region comprising CDR-L1 as depicted in SEQ ID NO: 633, CDR-L2 as depicted in SEQ ID NO: 634, CDR-L3 as depicted in SEQ ID NO: 635;
b) an antibody or protein comprising a domain which binds to CD3 on the surface of a T cell, wherein said domain comprises a VH region as depicted in SEQ ID NO: 639, and a VL region as depicted in SEQ ID NO: 641;
c) a protein comprising a domain which binds to CD3 on the surface of a T cell, wherein said domain comprises the amino acid sequence as depicted in SEQ ID NO: 642; or
d) a protein having the amino acid sequence as depicted in SEQ ID NO: 661.

It is also envisaged that the protein of the present invention binds to the same epitope of CD3 as:
a) an antibody or protein comprising a domain which binds to CD3 on the surface of a T cell, wherein said domain comprises a VH region comprising CDR-H1 as depicted in SEQ ID NO: 636, CDR-H2 as depicted in SEQ ID NO: 637, and CDR-H3 as depicted in SEQ ID NO: 638, and a VL region comprising CDR-L1 as depicted in SEQ ID NO: 633, CDR-L2 as depicted in SEQ ID NO: 634, CDR-L3 as depicted in SEQ ID NO: 635;
b) an antibody or protein comprising a domain which binds to CD3 on the surface of a T cell, wherein said domain comprises a VH region as depicted in SEQ ID NO: 639, and a VL region as depicted in SEQ ID NO: 641;
c) a protein comprising a domain which binds to CD3 on the surface of a T cell, wherein said domain comprises the amino acid sequence as depicted in SEQ ID NO: 642; or
d) a protein having the amino acid sequence as depicted in SEQ ID NO: 661.

It is furthermore envisaged that the protein of the present invention comprises a polypeptide having an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530. It is envisaged that the protein of the present invention comprises a polypeptide having an amino acid sequence as depicted in SEQ ID NO: 180.

It is also envisaged that the protein of the present invention comprises or consists of a polypeptide which has an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530, and which is linked at its N-terminus or at its C-terminus with a protein purification tag, preferably via a peptide bond (amide bond). The linking of the protein purification tag at the C-terminus of the polypeptide is preferred. It is envisaged that the protein purification tag is a short peptide. For example, the length of the short peptide may be 2-30 amino acids, 4-25 amino acids, 5-20 amino acids or 6-19 amino acids. Examples of protein purification tags include, but are not limited to, AU1 epitope (e.g. as depicted in SEQ ID NO: 666), AU5 epitope (e.g. as depicted in SEQ ID NO: 667), T7-tag (e.g. as depicted in SEQ ID NO: 668), V5-tag (e.g. as depicted in SEQ ID NO: 669), B-tag (e.g. as depicted in SEQ ID NO: 670), E2 epitope (e.g. as depicted in SEQ ID NO: 671), FLAG epitope/FLAG tag (e.g. as depicted in SEQ ID NO: 672), Glu-Glu tag (e.g. as depicted in SEQ ID NOs: 673 or 674), HA tag. Histidine affinity tag (e.g. as depicted in SEQ ID NO: 675), HSV epitope (e.g. as depicted in SEQ ID NO: 676), KT3 epitope (e.g. as depicted in SEQ ID NO: 677), Myc epitope (e.g. as depicted in SEQ ID NO: 678), polyarginine tag (5-6 Arg residues), polyaspartate tag (5-16 Asp residues), polyhistidine tag (2-10 His residues, usually 6 His residues, see e.g. SEQ ID NOs: 662-665), polyphenylalanine tag (usually 11 Phe residues), S1 tag (e.g. as depicted in SEQ ID NO: 679), S-tag (e.g. as depicted in SEQ ID NO: 680), Strep-tag (e.g. as depicted in SEQ ID NOs: 681 or 682), universal tag (e.g. as depicted in SEQ ID NO: 683), VSV-G (e.g. as depicted in SEQ ID NO: 684), Protein C (e.g. as depicted in SEQ ID NO: 685), and Protein A. A histidine tag is preferred, especially a 6× His tag (SEQ ID NO: 663).

It is also envisaged that the protein of the present invention binds to the same epitope of BCMA as:
a) an antibody or protein comprising a domain which binds to BCMA on the surface of a target cell, wherein said domain comprises a VH region comprising CDR-H1 as depicted in SEQ ID NO: 171, CDR-H2 as depicted in SEQ ID NO: 172, and CDR-H3 as depicted in SEQ ID NO: 173, and a VL region comprising CDR-L1 as depicted in SEQ ID NO: 174, CDR-L2 as depicted in SEQ ID NO: 175, and CDR-L3 as depicted in SEQ ID NO: 176;
b) an antibody or protein comprising a domain which binds to BCMA on the surface of a target cell, wherein said domain comprises a VH region as depicted in SEQ ID NO: 177, and a VL region as depicted in SEQ ID NO: 178;
c) a protein comprising a domain which binds to BCMA on the surface of a target cell, wherein said domain comprises the amino acid sequence as depicted in SEQ ID NO: 179; or d) a protein having the amino acid sequence as depicted in SEQ ID NO: 661.

Whether or not an antibody, binding domain or binding protein comprising such binding domain binds to the same epitope of BCMA/BCMA on the surface of a target cell as another given antibody, binding domain or binding protein can be measured by different analyses, e.g. by epitope mapping with chimeric or mutated BCMA molecules, as described in WO 2013/072406. Another possibility to identify the epitope within a target is an Alanine scanning assay (see e.g. Morrison K L & Weiss G A. Curr Opin Chem Biol. 2001 June; 5 (3): 302-7), where each residue within the target (here: BCMA) to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired. Alanine scanning is usually accomplished by site-directed mutagenesis or randomly by creating a PCR library. Furthermore, computational methods to estimate thermodynamic parameters based on theoretical alanine substitutions have been developed. The data can be tested by IR, NMR Spectroscopy, mathematical methods, bioassays, etc. The same analysis can of course be applied for other targets such as CD3.

It is also envisaged that the protein of the present invention competes for binding to BCMA with:
a) an antibody or protein comprising a domain which binds to BCMA on the surface of a target cell, wherein said domain comprises a VH region comprising CDR-H1 as depicted in SEQ ID NO: 171, CDR-H2 as depicted in SEQ ID NO: 172, and CDR-H3 as depicted in SEQ ID NO: 173, and a VL region comprising CDR-L1 as depicted in SEQ ID NO: 174, CDR-L2 as depicted in SEQ ID NO: 175, and CDR-L3 as depicted in SEQ ID NO: 176;
b) an antibody or protein comprising a domain which binds to BCMA on the surface of a target cell, wherein said domain comprises a VH region as depicted in SEQ ID NO: 177, and a VL region as depicted in SEQ ID NO: 178;
c) a protein comprising a domain which binds to BCMA on the surface of a target cell, wherein said domain comprises the amino acid sequence as depicted in SEQ ID NO: 179; or
d) a protein having the amino acid sequence as depicted in SEQ ID NO: 661.

Whether or not an antibody or protein according to the invention competes for binding to BCMA/BCMA on the surface of a target cell with another given antibody or protein according to the invention can be measured in a competition assay such as a competitive ELISA or a cell-based competition assay (using either cells that naturally express BCMA or cells that were stably or transiently transformed with BCMA). Avidin-coupled microparticles (beads) can also be used. Similar to an avidin-coated ELISA plate, when reacted with a biotinylated protein, each of these beads can be used as a substrate on which an assay can be performed. Antigen is coated onto a bead and then precoated with the first antibody. The second antibody is added, and any additional binding is determined. Read-out occurs via flow cytometry. The term “competes for binding”, in the present context, means that competition occurs between the two tested antibodies of at least 50%, at least 60%, at least 70%, at least 80% or at least 90%, as determined by any one of the assays disclosed above. The percentage refers to the reduction of the binding of the second antibody, occurring in the presence of the first antibody, as compared to a control assay where the first antibody is absent or replaced by an irrelevant (non-binding) antibody and the second antibody hence binds without any competition. The same analysis can be applied for other targets such as CD3.

The protein described herein comprises a third domain which extends or enhances the half-life (or “serum half-life”) of the protein. Examples for domains which extend the serum half-life of the proteins of the invention include peptides, polypeptides, proteins or domains of proteins, which are fused or otherwise attached to the proteins. The group of peptides, polypeptides, proteins or protein domains includes peptides binding to other proteins with preferred pharmacokinetic profile in the human body such as peptides binding to serum albumin (see WO 2009/127691). A further concept for half-life extension includes the fusion to an anti-albumin binding domain such as a single domain anti-albumin antibody. An alternative concept of such half-life extending peptides includes peptides binding to the neonatal Fc receptor (FcRn, see WO 2007/098420), which can also be used for the proteins of the present invention. The concept of attaching larger protein domains, polypeptides or complete proteins includes the fusion of human serum albumin, variants or mutants of human serum albumin (see WO 2011/051489, WO 2012/059486, WO 2012/150319, WO 2013/135896, WO 2014/072481, WO 2013/075066) or domains thereof, as well as the fusion of an immunoglobulin constant region (Fc domain) and variants thereof. Such variants of Fc domains are called Fc-based domains and may be further optimized/modified in order to allow the desired pairing of dimers or multimers, to abolish Fc receptor binding (e.g. to avoid ADCC or CDC) or for other reasons. A further concept known in the art to extend the half-life of substances or molecules in the human body is the pegylation of those molecules (such as the proteins of the present invention).

In one embodiment, the proteins according to the invention are linked (e.g. via peptide bond) with a fusion partner (such as a protein, polypeptide or peptide) for the purpose of extending the construct's serum half-life. These fusion partners (representing the “third domain”) can be selected from human serum albumin (“HSA” or “HALB”) as wells as sequence variants thereof, peptides binding to HSA, peptides binding to FcRn (“FcRn BP”), or constructs comprising an (antibody or immunoglobulin derived) Fc region. In general, the fusion partners providing for half-life extension may be linked to the N-terminus or to the C-terminus of the proteins according to the invention, either directly (e.g. via peptide bond) or through a peptide linker such as (GGGGS)n (wherein “n” is an integer of 2 or greater, e.g. 2 or 3 or 4). Suitable peptide linkers are depicted in SEQ ID NOs: 686-694. The third domain may also be located between the first and the second domain.

According to a further embodiment, the third domain of the protein of the invention comprises two polypeptide monomers, each comprising a hinge, a CH2 domain and a CH3 domain, preferably in said order from the N- to the C-terminus. It is envisaged that said two polypeptide monomers can be fused to each other via a peptide linker. In one embodiment, the third domain comprises in an N-terminal to C-terminal order: “first polypeptide monomer-linker-second polypeptide monomer” or “hinge-CH2-CH3-linker-hinge-CH2-CH3”. Amino acid sequences that can be used for said third domain are depicted in SEQ ID NOs: 700-707. Each of said polypeptide monomers can have an amino acid sequence that is selected from the group consisting of SEQ ID NOs: 708-715, or that is at least 90% identical to those sequences. In another embodiment, the first domain and the second domain of the protein of the invention are fused to the third domain via a peptide linker which is for example selected from the group consisting of SEQ ID NO: 686, 687, 688, 689, 690, 691, 692, 693 or 694.

In line with the present invention, a “hinge” is an IgG hinge region. This region can be identified by analogy using the Kabat numbering, see e.g. Kabat positions 223-243. In line with the above, the minimal requirement for a “hinge” are the amino acid residues corresponding to the IgG₁ sequence stretch of D231 to P243 according to the Kabat numbering. The terms “CH2” and “CH3” (or “CH2 domain” and “CH3 domain”) refer to the immunoglobulin heavy chain constant regions 2 and 3. These regions can as well be identified by analogy using the Kabat numbering, see e.g. Kabat positions 244-360 for CH2 and Kabat positions 361-478 for CH3. It is understood that there is some variation between the immunoglobulins in terms of their IgG₁ Fc region, IgG₂ Fc region, IgG₃ Fc region, IgG₄ Fc region, IgM Fc region, IgA Fc region, IgD Fc region and IgE Fc region (see, e.g., Padlan, Molecular Immunology, 31 (3), 169-217 (1993)). The term Fc region refers to the last two heavy chain constant regions of IgA. IgD, and IgG₁ and the last three heavy chain constant regions of IgE and IgM. The Fc region can also include the flexible hinge N-terminal to these domains. For IgA and IgM, the Fc region may include the J chain. For IgG, the Fc region comprises immunoglobulin domains CH2 and CH3 and the hinge. Although the boundaries of the Fc region of an immunoglobulin may vary, an example for a human IgG heavy chain Fc portion comprising a functional hinge, CH2 domain and CH3 domain can be defined e.g. to comprise residues D231 (of the hinge domain) to P476 (of the C-terminus of the CH3 domain), or D231 to L476, respectively, for IgG4, wherein the numbering is according to Kabat.

The protein of the invention may hence comprise in an N- to C-terminal order:
(a) the first domain;
(b) a peptide linker having an amino acid sequence selected from the group consisting of SEQ ID NOs: 687, 693 and 694;
(c) the second domain;
(d) a peptide linker having an amino acid sequence selected from the group consisting of SEQ ID NO: 686, 687, 688, 689, 690, 691, 692, 693 or 694;
(e) the first polypeptide monomer of the third domain (comprising a hinge, a CH2 domain and a CH3 domain);
(f) a peptide linker having an amino acid sequence selected from the group consisting of SEQ ID NOs: 695, 696, 697, 698 or 699; and
(g) the second polypeptide monomer of the third domain (comprising a hinge, a CH2 domain and a CH3 domain).

It is also envisaged that the protein of the invention comprises in an N- to C-terminal order:
f) the first domain having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529; wherein the peptide linker comprised within those sequences and having SEQ ID NO: 694 can be replaced by any one of SEQ ID NOs: 686-693 and 695-699;
g) a peptide linker having an amino acid sequence selected from the group consisting of SEQ ID NOs: 687, 693 and 694;
h) the second domain having an amino acid sequence selected from the group consisting of SEQ ID NOs: 540, 541, 552, 553, 563, 564, 574, 575, 586, 587, 597, 598, 608, 609, 619, 620, 631, 632, 642, 643, and 646; wherein the peptide linker comprised within those sequences and having SEQ ID NO: 694 can be replaced by any one of SEQ ID NOs: 686-693 and 695-699;
i) a peptide linker having an amino acid sequence selected from the group consisting of SEQ ID NOs: 686, 687, 688, 689, 690, 691, 692, 693, and 694; and
j) the third domain having an amino acid sequence selected from the group consisting of SEQ ID NOs: 700-707.

Hence, in one embodiment, the protein of the present invention comprises in an N- to C-terminal order:
(a) a polypeptide having an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530;
(b) a linker having an amino acid sequence as depicted in SEQ ID NO: 686; and
(c) the third domain having an amino acid sequence as depicted in SEQ ID NO: 700.

A preferred protein of the present invention comprises or consists of a polypeptide having the amino acid sequence as depicted in SEQ ID NO: 661.

While such protein may be in a single-chain format, the protein to be administered according to the present invention may also have the format of an IgG and hence have an IgG-like structure, i.e. a structure which is similar to the one of a full-length immunoglobulin as it occurs naturally. In one embodiment, the protein may comprise or consist of two antibody heavy chains (each one having a VH, a CH1, a CH2 and a CH3 domain) and two antibody light chains (each one having a VL and a CL domain) which assemble to an antibody-like structure. One arm may bind to BCMA (hence constituting the “first domain which binds to BCMA”), one arm may bind to CD3 (hence constituting the “second domain which binds to CD3”), and the Fc domain (comprising the CH2 and CH3 domains) constitutes the “third domain which extends the half-life of the protein”. This protein may also comprise glycosylations. Variations of such protein are also encompassed by the present invention, such as a format in which a binding arm does not comprise a light chain, but still binds to the respective target (BCMA or CD3), or a format in which a binding arm is bivalent for the respective target. Examples for such proteins are described inter alia in Harlow and Lane, Antibodies: A laboratory manual, CSHL Press (1988); Kontermann and Dübel, Antibody Engineering, Springer, 2nd ed. 2010; and Little, Recombinant Antibodies for Immunotherapy, Cambridge University Press 2009.

It is hence an object of the invention that
a) the protein is a single chain protein (or “single chain polypeptide”, i.e. it consists of one polypeptide chain) or consists of two, three or four polypeptide chains,
b) the first domain comprises an immunoglobulin heavy chain variable region (VH1) and an immunoglobulin light chain variable region (VL1),
c) the second domain comprises an immunoglobulin heavy chain variable region (VH2) and an immunoglobulin light chain variable region (VL2), and/or
d) the third domain comprises two immunoglobulin hinge regions, two CH2 domains and two CH3 domains.

The binding domains that specifically bind to BCMA and CD3 can be derived from known antibodies to these antigens or from new antibodies or antibody fragments obtained by de novo immunization methods using the antigen proteins or fragments thereof, by phage display, or other methods known in the art. The antibodies from which the binding domains for the proteins are derived can be monoclonal antibodies, recombinant antibodies, chimeric antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

In some embodiments, the protein of the invention is an “isolated” or “substantially pure” protein. “Isolated” or “substantially pure”, when used to describe the proteins disclosed herein, means a protein that has been identified, separated and/or recovered from a component of its production environment. Preferably, the protein is free or substantially free of association with all other components from its production environment. Contaminant components of its production environment, such as that resulting from recombinant transfected cells, are materials that could interfere with diagnostic or therapeutic uses for the protein, and may include enzymes, hormones, and other proteinaceous or non-proteinaceous compounds. It is understood that the isolated or substantially pure protein may constitute from 80% to 99.9% by weight of the total protein/polypeptide content in a given sample, depending on the circumstances. The desired protein may be produced at a significantly higher concentration through the use of an inducible promoter or high expression promoter. The definition includes the production of a binding protein in a wide variety of organisms and/or host cells that are known in the art. In certain embodiments, the protein will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver staining. Usually, however, an isolated protein will be prepared by at least one purification step.

The protein of the present invention is typically formulated in a pharmaceutical composition or a formulation. Materials of a pharmaceutical composition are usually formulated in concentrations that are acceptable for the site of administration. Formulations and compositions thus may be designed in accordance with the invention for delivery by any suitable route of administration.

As used herein, the term “pharmaceutical composition” relates to a composition which is suitable for administration to a patient, preferably a human patient. A preferred pharmaceutical composition of this invention comprises one or a plurality of the protein(s) of the invention, usually in a therapeutically effective amount. The pharmaceutical composition may further comprise suitable formulations of one or more (pharmaceutically effective) carriers, stabilizers, excipients, diluents, solubilizers, surfactants, emulsifiers, preservatives and/or adjuvants. Acceptable constituents of the composition are typically nontoxic to recipients at the dosages and concentrations employed. Pharmaceutical compositions of the invention include, but are not limited to, liquid, frozen, and lyophilized compositions. If the pharmaceutical composition has been lyophilized, the lyophilized material is reconstituted in an appropriate liquid prior to administration. The lyophilized material may e.g. be reconstituted in bacteriostatic water for injection (BWFI), physiological saline, phosphate buffered saline (PBS), or the same formulation the protein had been in prior to lyophilization.

The compositions may comprise a pharmaceutically acceptable carrier. In general, as used herein, “pharmaceutically acceptable carrier” means any and all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the protein of the invention and further one or more excipients. Excipients can be used for a wide variety of purposes, such as adjusting physical, chemical, or biological properties of formulations, such as adjustment of viscosity, and or processes of the invention to improve effectiveness and/or to stabilize such formulations and processes against degradation and spoilage e.g. due to stresses that occur during manufacturing, shipping, storage, pre-use preparation, administration, and thereafter. Excipients should in general be used in their lowest effective concentrations.

In certain embodiments, the pharmaceutical composition may also contain formulation materials/substances for the purpose of modifying, maintaining or preserving certain characteristics of the composition such as the pH, osmolarity, viscosity, clarity, color, isotonicity, odor, sterility, stability, rate of dissolution or release, adsorption or penetration (see, Remington's Pharmaceutical Sciences, 18″ Edition, 1990, Mack Publishing Company).

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within ±15%, more preferably within ±10%, and most preferably within ±5% of a given value or range. It also includes the concrete value, e.g., “about 50” includes the value “50”.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein, “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

It should be understood that the above description and the below examples provide exemplary arrangements, but the present invention is not limited to the particular methodologies, techniques, protocols, material, reagents, substances, etc., described herein and as such can vary. The terminology used herein is for the purpose of describing particular embodiments only, and is not intended to limit the scope of the present invention, which is defined solely by the claims. Aspects of the invention are provided in the independent claims. Some optional features of the invention are provided in the dependent claims.

All publications and patents cited throughout this specification (including all patents, patent applications, scientific publications, manufacturer's specifications, instructions, etc.), whether supra or infra, are hereby incorporated by reference in their entireties. Nothing herein is to be construed as an admission that the invention is not entitled to antedate such disclosure by virtue of prior invention. To the extent the material incorporated by reference contradicts or is inconsistent with this specification, the specification will supersede any such material. It is understood that the disclosed invention is not limited to the particular methodology, protocols and materials described as these can vary. It is also understood that the terminology used herein is for the purposes of describing particular embodiments only and is not intended to limit the scope of the appended claims.

A better understanding of the present invention and of its advantages will be obtained from the following example, offered for illustrative purposes only. The examples are not intended and should not be construed as to limit the scope of the present invention in any way.

### Example

Background: AMG 701, an HLE BITER molecule which binds BCMA on Multiple Myeloma (MM) cells and CD3 on T cells, has activity in MM preclinical models. Objectives of this first-in-human study include evaluating safety and estimating the maximum tolerated dose (MTD) of AMG 701 in patients with relapsed/refractory (R/R) Multiple Myeloma (NCT03287908).

Methods: Weekly short-term IV infusions of AMG 701 were administered in 4-week cycles for as long as patients benefitted, first to single-patient cohorts (5, 15, 45 μg), then cohorts of ≥3 patients (140, 400, 800, 1200 μg), and possibly up to 10 patients in higher-dose cohorts (1600, 3000, 4500, 6500, 9000, 12000, 180000 μg). Eligible patients had MM relapsed after or intolerant to ≥2 lines with a proteasome inhibitor, IMiD, and anti-CD38 MAb (where approved and available). Excluded were patients with solely extramedullary disease (allowed in subsequent dose expansion), CNS involvement, prior BCMA-directed therapy, and immunosuppressive therapy. After a grade 3 dose-limiting toxicity (DLT) of cytokine release syndrome (CRS) at 1600 μg, for all target doses ≥1200 μg, an initial run-in dose of 800 μg was added, followed by step-up to the target dose in one or two steps, with the target dose being administered weekly. MRD was defined as <1/10⁵ cells, as measured per the FDA-authorized next generation sequencing assay

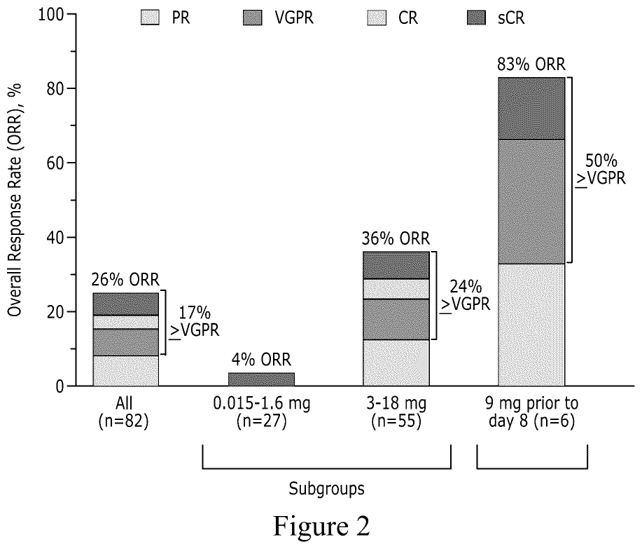

Results (as of the data cut-off in Q3-2020): Median age was 64 years, 25% of patients had extramedullary disease, patients had a median of six prior lines of therapy, 82% had a prior stem cell transplant. 93% were triple-exposed and 62% were triple refractory to PI, IMiD and anti-CD38 Ab. No anti-AMG 701 antibodies were detected. Of all 82 patients treated, the overall response rate (ORR) was 26%, with 17% having a response ≥VGPR. Of the 55 patients treated with a target dose from 3 mg to 18 mg, the ORR increased to 36%. In the group of patients receiving a target dose of 9 mg prior to day 8 (n=6), there were 5/6 (83%) responses; 80% (4/5) of these responders were triple refractory, and 50% had a response ≥VGPR. See FIG. 2. Response data from 12 mg target dose prior to day 8 are under evaluation. Of the four patients having received a target dose of 18 mg at d8 with a d1 run-in dose of 800 μg, so far one has shown a PR and one a VGPR, but final data are still under evaluation. A further group of patients receiving an additional dose of 9 mg at d3 (i.e. d1-800 μg/d3-9 mg/d8-18 mg, followed by weekly dosing of 18 mg target dose) is still under evaluation.

TABLE 1
The table shows a cohort with a target dose of 9 mg. All patients
shown in the table received a run-in dose of 800 μg on d 1 prior
to receiving the target dose on d 3 and d 8. 5/6 patients receiving
this regimen showed a response. The table does not include four
patients who received a run-in dose of 800 μg on d 1 followed
by the target dose of 9 mg on d 8. sCR: Stringent Complete Response.

| Cohort: | # of | Day to | | | |
|---|---|---|---|---|---|
| target dose | patients | target | Best | Any response (months) | |
| (μg)* | at dose | dose | response | Onset | Duration |
| 13:9000 | 6 | 3 | sCR | 0.95 | 5.59 |
| | | | PR | 1.88 | 2.80 |
| | | | VGPR | 0.95 | 3.52 |
| | | | VGPR | 0.99 | 0.03 |
| | | | PR | 0.95 | 2.11 |

The overall study demonstrated:
A manageable safety profile: All Grade 3 CRS events were reversible with a median duration of 2 days; 50% of grade 3 CRS designations were driven by transient LFT increases
Encouraging activity, with an 83% ORR in heavily pretreated patients—4/5 responders were triple refractory—at a dosing regimen with a 9 mg target dose reached prior to day 8
Responses lasting up to 26 months
MRD was tested in 6 patients at doses of 0.8 mg (n=1), 3 mg (n=1), 4.5 mg (n=1), 6.5 mg (n=2), 12 mg (n=1) with best responses of 3 sCRs, 2 CRs, 1 VGPR; all but one were MRD negative, with MRD negativity lasting up to 22 months
An IV PK profile supportive of once-weekly dosing

### Updated Results:

1. Based on data gathered from a total of 22 separate AMG 701 dose escalation cohorts (including no step dosing, as well as day 1→day 8 step dosing and day 1→day 3→(day 5)→day 8 step dosing), the dosing schedule administering a second dose on day 3 was considered very safe, with seven cohorts currently treating patients with this schedule (see Table 2).
2. As of 19 Oct. 2021, 50 patients have been treated with this dosing schedule (i.e. second dose on day 3):
   a. 20 patients are still on treatment
   b. The median age is 66 years and the majority are male (54%), with similar baseline characteristics as previously reported data
   c. The ORR for the treated patients is 54%, while for response evaluable patients it is 60%
   d. The BOR distribution is displayed in Table 2 below
   e. Although the data in the last two cohorts (15B and 15C) is still immature, the safety profile appears to be consistent with previously reported data. The CRS (cytokine release syndrome) incidence has improved, especially within cohort 15C (still enrolling): No grade 3 or higher has been reported for the last two cohorts (15B and 15C).

TABLE 2
Best Overall Response (BOR) distribution (number “n” and percentage)
of cohorts with a day 1/day 3/(day 5)/day 8 administration regimen.

| | | | | | | | Cohort | | |
|---|---|---|---|---|---|---|---|---|---|
| | | | | Cohort | Cohort | Cohort | 15C: | | |
| | Cohort | Cohort | Cohort | 14A: | 15A: | 15B: | 800 μg/ | Total | |
| | 12 ext: | 13 ext: | 14 ext: | 800 μg/ | 800 μg/ | 800 μg/ | 4.5 mg/ | for | |
| | 800 μg/ | 800 μg/ | 800 μg/ | 9 mg/ | 9 mg/ | 6 mg/ | 9 mg/ | 2^{nd} dose | Overall |
| | 6.5 mg | 9 mg | 12 mg | 12 mg | 18 mg | 18 mg | 18 mg | on d 3 | Total |
| | (N = 6) | (N = 6) | (N = 9) | (N = 4) | (N = 8) | (N = 10) | (N = 7) | (N = 50) | (N = 116) |
| sCR | 1 (16.7) | 1 (16.7) | 1 (11.1) | 0 (0.0) | 1 (12.5) | 1 (10.0) | 0 (0.0) | 5 (10.0) | 10 (8.6) |
| CR | 1 (16.7) | 0 (0.0) | 3 (33.3) | 2 (50.0) | 3 (37.5) | 0 (0.0) | 1 (14.3) | 10 (20.0) | 12 (10.3) |
| VGPR | 0 (0.0) | 1 (16.7) | 0 (0.0) | 0 (0.0) | 2 (25.0) | 3 (30.0) | 0 (0.0) | 6 (12.0) | 10 (8.6) |
| PR | 0 (0.0) | 3 (50.0) | 1 (11.1) | 0 (0.0) | 0 (0.0) | 1 (10.0) | 1 (14.3) | 6 (12.0) | 9 (7.8) |
| MR | 0 (0.0) | 0 (0.0) | 0 (0.0) | 0 (0.0) | 0 (0.0) | 0 (0.0) | 0 (0.0) | 0 (0.0) | 1 (0.9) |
| SD | 3 (50.0) | 0 (0.0) | 3 (33.3) | 0 (0.0) | 2 (25.0) | 3 (30.0) | 1 (14.3) | 12 (24.0) | 36 (31.0) |
| PD | 1 (16.7) | 1 (16.7) | 1 (11.1) | 0 (0.0) | 0 (0.0) | 2 (20.0) | 1 (14.3) | 6 (12.0) | 18 (15.5) |
| NE | 0 (0.0) | 0 (0.0) | 0 (0.0) | 2 (50.0) | 0 (0.0) | 0 (0.0) | 3 (42.9) | 5 (10.0) | 20 (17.2) |

sCR = stringent complete response.
CR = complete response.
VGPR = very good partial response.
PR = partial response.
MR = minimal response.
SD = stable disease.
PD = progressive disease.
NE = not evaluable.

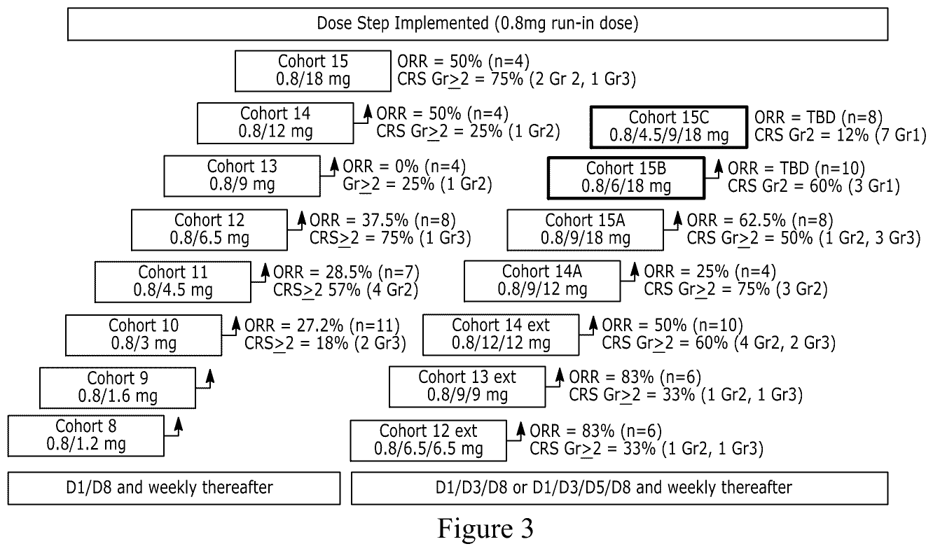

See also FIG. 3 for the step dosing cohorts and their ORR and CRS results.

The IMWG response criteria for a complete response (CR, see also http://imwg.myeloma.org/international-myeloma-working-group-imwg-uniform-response-criteria-for-multiple-myeloma/) are:
Negative M protein immunofixation on the serum and urine,
Disappearance of any soft tissue plasmacytomas, and
<5% plasma cells in bone marrow

The IMWG response criteria for a partial response (PR) are:
M protein electrophoresis: ≥50% reduction of serum M-protein and reduction in 24 hours urinary M-protein by ≥90% or to <200 mg/24 h
Free light chains (FLC): If the serum and urine M protein are unmeasurable, a ≥50% decrease in the difference between involved and uninvolved FLC levels is required in place of the M-protein criteria
If serum and urine M protein are not measurable, and serum free light assay is also not measurable: ≥50% reduction in plasma cells is required in place of M protein, provided baseline bone marrow plasma cell percentage was ≥30%.
In addition to the above listed criteria, if present at baseline, a ≥50% reduction in the size of soft tissue plasmacytomas is also required

The IMWG response criteria for a very good partial response (VGPR) are:
Serum and urine M protein detectable by immunofixation but not on electrophoresis or ≥90% reduction in serum M-protein plus urine M-protein level <100 mg/24 h

1. A method of treating or ameliorating a BCMA-positive neoplasm in a subject, the method comprising administering to the subject a first cycle of a target dose from 12.5 mg/day to about 24 mg/day of a protein comprising a first domain which binds to BCMA, a second domain which binds to CD3, and a third domain which extends the half-life of the protein.

2. The method of claim 1, wherein the first cycle further comprises:
administering a first dose of the protein on day 1;
administering a second dose of the protein on a day after day 1 and before day 8, wherein the second dose exceeds the first dose; and
optionally administering a third dose of the protein on a day after the day of administration of the second dose and before the day of administration of the target dose, wherein the third dose exceeds the second dose.

3. The method of claim 2, wherein
a) the second dose is administered on day 3 or day 4, preferably on day 3;
b) the third dose is administered on day 5, day 6, or day 7; and/or
c) the target dose is administered on a day from day 6 to day 10,
preferably on a day from day 7 to day 9, more preferably on day 8 and optionally on day 15 (+/− one or two days) and day 22 (+/− one or two days).

4. The method of claim 2, wherein
a) the first dose is from about 800 μg/day to about 1200 μg/day, from about 800 μg/day to about 1100 μg/day, from about 800 μg/day to about 1000 μg/day or from about 800μ g/day to about 900 μg/day;
b) the second dose is from about 4 mg/day to about 12.5 mg/day, from about 4.5 mg/day to about 12 mg/day, from about 4 mg/day to about 10 mg/day, from about 4.5 mg/day to about 9 mg/day, from about 4 mg/day to about 8 mg/day, from about 4 mg/day to about 7 mg/day, from about 4 mg/day to about 6.5 mg/day, from about 4.5 mg/day to about 6 mg/day, from about 7 mg/day to about 18 mg/day, from about 7.5 mg/day to about 15 mg/day, from about 8 mg/day to about 12 mg/day, from about 8.5 mg/day to about 10 mg/day or from about 9 mg/day to about 9.5 mg/day;
c) the third dose is from about 7 mg/day to about 12 mg/day, from about 8 mg/day to about 10 mg/day or about 9 mg/day; and/or
d) the target dose is from about 14 mg/day to about 22 mg/day, from about 15 mg/day to about 21 mg/day, from about 16 mg/day to about 20 mg/day, from about 17 mg/day to about 19 mg/day, or about 18 mg/day.

5. The method of claim 2, wherein
a) the second dose is from about 4 mg/day to about 10 mg/day, preferably from about 4 mg/day to about 7 mg/day, and is administered on day 3;
b) the third dose is from about 8 mg/day to about 10 mg/day and is administered on day 5; and/or
c) the target dose is from about 16 mg/day to about 20 mg/day and is administered on day 8.

6-13. (canceled)

14. The method of claim 1, wherein the protein is administered in a second cycle and optionally in further subsequent cycles at the target dose.

15. The method of claim 14, wherein the second cycle and optionally the further subsequent cycles comprise
administering the protein at the target dose on day 1, day 8 (+/− one or two days), day 15 (+/− one or two days) and day 22 (+/− one or two days).

16. The method of claim 1, wherein one cycle has about 25 to about 30 days, about 26 to about 29 days, about 27 to about 29 days, or about 28 days.

17. The method of claim 1, wherein the first cycle comprises:
administering a first dose of the protein of about 800 μg/day to about 1000 μg/day, preferably about 800 μg/day;
administering a second dose of the protein of about 4 mg/day to about 10 mg/day, preferably about 4 mg/day to about 7 mg/day or 4.5 mg/day to 6 mg/day;
optionally administering a third dose of the protein of about 8 mg/day to about 10 mg/day, preferably about 9 mg/day; and
administering the target dose of the protein of about 14 mg/day to about 22 mg/day, preferably about 16 mg/day to about 20 mg/day, such as 18 mg/day.

18. The method of claim 17, wherein the first dose is administered on day 1, the second dose is administered on day 3 or day 4, preferably on day 3, the optional third dose is administered on day 5 or day 6, preferably on day 5, and the target dose is administered on day 8 (+/− one day), day 15 (+/− one day) and day 22 (+/− one day), preferably on day 8, day 15 and day 22.

19. The method of claim 2, wherein the first dose of 800 μg/day is administered on day 1, the second dose of 6 mg/day is administered on day 3, and the target dose of 18 mg/day is administered on day 8, day 15 and day 22.

20. The method of claim 2, wherein the first dose of 800 μg/day is administered on day 1, the second dose of 4.5 mg/day is administered on day 3, the third dose of 9 mg/day is administered on day 5, and the target dose of 18 mg/day is administered on day 8, day 15 and day 22.

21. The method of claim 1, wherein the protein is administered intravenously, preferably via intravenous bolus injection, bolus infusion or short-term intravenous infusion.

22. The method of claim 1, wherein the BCMA positive neoplasm is selected from the group consisting of multiple myeloma, relapsed and/or refractory multiple myeloma, heavy chain multiple myeloma, light chain multiple myeloma, extramedullary myeloma, plasmacytoma, plasma cell leukemia, Waldenström's macroglobulinemia, and smoldering myeloma.

23. The method of claim 1, wherein
a) the protein is a single chain protein or consists of two, three or four polypeptide chains;
b) the first domain comprises an immunoglobulin heavy chain variable region (VH1) and an immunoglobulin light chain variable region (VL1);
c) the second domain comprises an immunoglobulin heavy chain variable region (VH2) and an immunoglobulin light chain variable region (VL2); and/or
d) the third domain comprises one or two immunoglobulin hinge regions, one or two CH2 domains and one or two CH3 domains.

24. The method of claim 1, wherein the protein competes for binding to BCMA with or binds to the same epitope of BCMA as:
a) an antibody or protein comprising a domain which binds to BCMA on the surface of a target cell, wherein said domain comprises a VH region comprising CDR-H1 as depicted in SEQ ID NO: 171, CDR-H2 as depicted in SEQ ID NO: 172, and CDR-H3 as depicted in SEQ ID NO: 173, and a VL region comprising CDR-L1 as depicted in SEQ ID NO: 174, CDR-L2 as depicted in SEQ ID NO: 175, and CDR-L3 as depicted in SEQ ID NO: 176;
b) an antibody or protein comprising a domain which binds to BCMA on the surface of a target cell, wherein said domain comprises a VH region as depicted in SEQ ID NO: 177, and a VL region as depicted in SEQ ID NO: 178;
c) a protein comprising a domain which binds to BCMA on the surface of a target cell, wherein said domain comprises the amino acid sequence as depicted in SEQ ID NO: 179; or
d) a protein having the amino acid sequence as depicted in SEQ ID NO: 661.

25. The method of claim 1, wherein the protein competes for binding to CD3 with or binds to the same epitope of CD3 as:
a) an antibody or protein comprising a domain which binds to CD3 on the surface of a T cell, wherein said domain comprises a VH region comprising CDR-H1 as depicted in SEQ ID NO: 636, CDR-H2 as depicted in SEQ ID NO: 637, and CDR-H3 as depicted in SEQ ID NO: 638, and a VL region comprising CDR-L1 as depicted in SEQ ID NO: 633, CDR-L2 as depicted in SEQ ID NO: 634, CDR-L3 as depicted in SEQ ID NO: 635;
b) an antibody or protein comprising a domain which binds to CD3 on the surface of a T cell, wherein said domain comprises a VH region as depicted in SEQ ID NO: 639, and a VL region as depicted in SEQ ID NO: 641;
c) a protein comprising a domain which binds to CD3 on the surface of a T cell, wherein said domain comprises the amino acid sequence as depicted in SEQ ID NO: 642; or
d) a protein having the amino acid sequence as depicted in SEQ ID NO: 661.

26. The method of claim 1, wherein the first domain which binds to BCMA
a) comprises a VH region having an amino acid sequence selected from the group consisting of those depicted in SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 307, 317, 327, 337, 347, 357, 367, 377, 387, 397, 407, 417, 427, 437, 447, 457, 467, 477, 487, 497, 507, 517, and 527, preferably SEQ ID NO: 177;
b) comprises a VL region having an amino acid sequence selected from the group consisting of those depicted in SEQ ID NOs: 8, 18, 28, 38, 48, 58, 68, 78, 88, 98, 108, 118, 128, 138, 148, 158, 168, 178, 188, 198, 208, 218, 228, 238, 248, 258, 268, 278, 288, 298, 308, 318, 328, 338, 348, 358, 368, 378, 388, 398, 408, 418, 428, 438, 448, 458, 468, 478, 488, 498, 508, 518, and 528, preferably SEQ ID NO: 178; and/or
c) comprises or consists of a polypeptide having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529, preferably SEQ ID NO: 179.

27-28. (canceled)

29. The method of claim 1, wherein the second domain which binds to CD3
a) comprises a VL region having an amino acid sequence selected from the group consisting of those depicted in SEQ ID NO: 550, SEQ ID NO: 551, SEQ ID NO: 584, SEQ ID NO: 585, SEQ ID NO: 629 and SEQ ID NO: 630, preferably SEQ ID NO: 629;
b) comprises a VH region having an amino acid sequence selected from the group consisting of those depicted in SEQ ID NO: 537, SEQ ID NO: 538, SEQ ID NO: 548, SEQ ID NO: 549, SEQ ID NO: 560, SEQ ID NO: 561, SEQ ID NO: 571, SEQ ID NO: 572, SEQ ID NO: 582, SEQ ID NO: 583, SEQ ID NO: 594, SEQ ID NO: 595, SEQ ID NO: 605, SEQ ID NO: 606, SEQ ID NO: 616, SEQ ID NO: 617, SEQ ID NO: 627, SEQ ID NO: 628, SEQ ID NO: 639, SEQ ID NO: 640, and SEQ ID NO: 644, preferably SEQ ID NO: 639; and/or
c) comprises or consists of a polypeptide having an amino acid sequence selected from the group consisting of those depicted in SEQ ID NOs: 540, 541, 552, 553, 563, 564, 574, 575, 586, 587, 597, 598, 608, 609, 619, 620, 631, 632, 642, 643, and 646, preferably SEQ ID NO: 642.

30-31. (canceled)

32. The method of claim 1, wherein the protein comprises a polypeptide having an amino acid sequence selected from the group consisting of those depicted in SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, and 530.

33. The method of claim 1, wherein the protein comprises or consists of, in an N- to C-terminal order:
a) the first domain having an amino acid sequence selected from the group consisting of SEQ ID NOs: 9, 19, 29, 39, 49, 59, 69, 79, 89, 109, 129, 139, 149, 159, 169, 179, 189, 199, 209, 219, 229, 239, 249, 259, 269, 279, 289, 299, 309, 319, 329, 339, 349, 359, 369, 379, 389, 399, 409, 419, 429, 439, 449, 459, 469, 479, 489, 499, 519, and 529; wherein the peptide linker comprised within those sequences and having SEQ ID NO: 694 can be replaced by any one of SEQ ID NOs: 686-693 and 695-699;
b) a peptide linker having an amino acid sequence selected from the group consisting of SEQ ID NOs: 687, 693 and 694;
c) the second domain having an amino acid sequence selected from the group consisting of SEQ ID NOs: 540, 541, 552, 553, 563, 564, 574, 575, 586, 587, 597, 598, 608, 609, 619, 620, 631, 632, 642, 643, and 646; wherein the peptide linker comprised within those sequences and having SEQ ID NO: 694 can be replaced by any one of SEQ ID NOs: 686-693 and 695-699;
d) a peptide linker having an amino acid sequence selected from the group consisting of SEQ ID NOs: 686, 687, 688, 689, 690, 691, 692, 693, and 694; and
e) the third domain having an amino acid sequence selected from the group consisting of SEQ ID NOs: 700-707.

34. The method of claim 1, wherein the protein has a molecular weight of about 75 to about 200 kDa, about 80 to about 175 kDa, about 85 to about 150 kDa, about 90 to about 130 kDa, about 95 to about 120 kDa, and preferably about 100 to about 115 kDa or about 105 to about 110 kDa.

35. The method of claim 1, wherein the protein has an elimination half-life (T_{1/2}) of about 3 days to about 14 days, about 4 days to about 12 days, about 3 or 4 days to about 10 days, about 3 or 4 days to about 8 days, or about 5 to about 7 days, or about 6 days.
